(19) United States
(12) Patent Application Publication
(10) Pub. No.: US 2026/0102517 A1
(43) Pub. Date: Apr. 16, 2026

(54) ADENO-ASSOCIATED VIRUS (AAV) DIRECTED ANTIOXIDATIVE GENE THERAPY FOR THE PREVENTION, AMELIORATION, AND/OR TREATMENT OF HEARING LOSS

(71) Applicant: Rutgers, The State University of New Jersey, New Brunswick, NJ (US)

(72) Inventors: Todd MOWERY, Piscataway, NJ (US); P. Ashley WACKYM, New Brunswick, NJ (US)

(73) Assignee: Rutgers, The State University of New Jersey, New Brunswick, NJ (US)

(21) Appl. No.: 19/114,370

(22) PCT Filed: Sep. 22, 2023
(86) PCT No.: PCT/US2023/074824

§ 371 Date: Mar. 21, 2025

Related U.S. Application Data
(60) Provisional application No. 63/409,568, filed on Sep. 23, 2022.

(51) Int. Cl. A61K 48/00; A61K 38/44; A61P 27/16; C12N 15/86

(52) U.S. Cl. CPC A61K 48/0058; A61K 38/446; A61P 27/16; C12N 15/86; C12Y 115/01001; C12N 2750/14122; C12N 2750/14143; C12N 2750/14145; C12N 2750/14171

(57) ABSTRACT

The disclosure relates to compositions and methods comprising recombinant AAV vectors comprising polynucleotide sequences encoding superoxide dismutase (SOD) proteins. Also included are methods of treating or preventing acute, progressive, aging, and ototoxic hearing loss comprising said methods and compositions. Also included are methods of treating or preventing acute, progressive, aging, and ototoxic vestibular hair cell loss comprising said methods and compositions.

### CROSS REFERENCE TO RELATED APPLICATION

The present application claims priority under 35 U.S.C. § 119(e) to U.S. Provisional Patent Application No. 63/409,568, filed Sep. 23, 2022, which is hereby incorporated by reference in its entirety herein.

### BACKGROUND

Hearing loss is the third most common chronic health problem in the United States, with noise-induced hearing loss effecting nearly 1 out of every 4 adults. According to the World Health Organization (WHO), there are over half a billion people that suffer from some sort of hearing loss. Of these, a small percentage falls into the category of congenital, in which a genetic disorder causes unilateral or bilateral deafness at birth, or moderative to severe hearing loss through progressive hearing loss as the child develops. These syndromic and non-syndromic genetic disorders involve hundreds of genes with complex up and down stream interactions that limit gene therapy approaches. The majority of hearing loss arises from occupational or recreational noise, natural aging, and exposure to ototoxic agents such as chemotherapy or specific types of antibiotics. These share a common etiology, in that oxidative processes produce free radicals that must be eliminated by the hair cells, support cells, and the ganglion cells of the cochlea and vestibular end-organs of the inner ear before they cause damage that can lead to apoptosis and necrosis.

Noise-induced hearing loss is a function of duration of exposure and sound intensity/amplitude. Most people exposed to dangerous levels of noise (over 70%) do not wear hearing protection; however, noise-induced hearing loss (NIHL) can also occur in individuals using acoustic protection. Commercially available acoustic protection can reduce the sound intensity by 20 dB (foam ear plugs) or at most 37 dB (over the earmuffs), so prolonged exposure to dangerous levels of sound (120+15 dB) even with proper protection would eventually lead to inner ear injury and hearing loss.

Thus, both recreational and occupational noise exposure result in a widespread health issue. In fact, all active-duty military are exposed to dangerous levels of noise, and the majority will face a lifetime of hearing loss and hearing disorders (e.g., tinnitus, impaired speech understanding). There is also a higher rate of dementia in patients with untreated hearing loss. Over the last few decades much of the work surrounding treatment for NIHL has focused on regrowing lost hair cells: however, this has not led to a breakthrough. Small molecule research targeted at the hair cells of the inner ear showed promise; however, limitations of delivery and sustained action/efficacy has not led to any clinically useful FDA approved treatments. Much of the noise-induced damage comes from secondary effects of oxidative action after strong activation (loud noise exposure over a duration), leading to apoptosis and necrosis of the hair cells of the inner ear.

In aging individuals the probability of hearing loss increases with age. At age 65, one in three adults has some level of hearing loss, and this percentage drastically increases as individuals approach their 80s. This natural byproduct of aging is largely related to oxidative damage over time. It is impossible to avoid all noise exposure and a lifetime of experience and daily use accrues and eventually leads to the death of the cochlear hair cells, support cells and ganglion cells. As these levels reach critical threshold, we see the emergence of hearing loss and balance issues in the elderly. Superoxide dismutase, which is highly neuroprotective against the oxidative damage process naturally declines with aging. Thus, it is likely the loss of these protective enzymes with age that lead to sensitivity and susceptibility in the aging population.

Exposure to ototoxic agents is another common factor in the onset of hearing loss in both children and adults. For example, nearly 20 to 40 percent of individuals that must take chemotherapy to treat cancer will develop some permanent mild to moderate/severe hearing loss. The compounds used to treat the cancer are not readily removed by the inner ear cells and lead to cell damage and death through oxidative pathways. Another form of ototoxic inner ear cell damage comes from certain types of antibiotics. While no longer widely used in the United States, many countries continue to use inexpensive antibiotics, such as gentamicin, in their populations leading to damage of the cochlear and vestibular cells that lead to eventual hearing loss and balance issues. Gentamicin is more vestibulotoxic than cochleotoxic; however, the profile of antibiotic-induced toxicity varies by antibiotics.

Most of the hearing loss currently experienced by humans throughout the world has an etiological connection to oxidative processes that damage the cells of the inner ear. As such, there is a need for treatment strategies which can both protect against the development of hearing loss and halt the progression of hearing loss by targeting the overproduction of ROS in inner ear cells. The current invention addresses these needs.

### SUMMARY OF THE DISCLOSURE

As described herein, the present disclosure relates to compositions and methods comprising recombinant AAV vectors comprising polynucleotide sequences encoding superoxide dismutase (SOD) proteins. Also included are methods of treating, ameliorating, and/or preventing progressive hearing loss comprising such methods and compositions.

As such, in one aspect, the disclosure includes a polynucleotide encoding an adeno-associated virus (AAV) vector. In certain embodiments, the AAV vector comprises a first AAV inverted terminal repeat (ITR) nucleic acid sequence. In certain embodiments, the AAV vector comprises a nucleic acid encoding a superoxide dismutase (SOD) protein operably linked to a promoter. In certain embodiments, the AAV vector comprises a second AAV inverted terminal repeat (ITR) nucleic acid sequence. In certain embodiments, the AAV vector is targeted to a cell of the inner ear.

The disclosure further includes a recombinant adeno-associated virus (AAV) vector. In certain embodiments, the AAV vector comprises a first AAV inverted terminal repeat (ITR) nucleic acid sequence. In certain embodiments, the AAV vector comprises a second AAV inverted terminal repeat (ITR) nucleic acid sequence. In certain embodiments, the AAV vector comprises a polynucleotide sequence encoding a superoxide dismutase (SOD) protein operably linked to a promoter flanked by the first AAV inverted terminal repeat (ITR) sequence and the second AAV ITR. In certain embodiments, the AAV vector has a specificity for a cell of the inner ear.

The disclosure further includes a cell comprising an adeno-associated virus (AAV) vector. In certain embodiments, the AAV vector comprises a first AAV inverted terminal repeat (ITR) nucleic acid sequence. In certain embodiments, the AAV vector comprises a second AAV inverted terminal repeat (ITR) nucleic acid sequence. In certain embodiments, the AAV vector comprises a nucleic acid encoding a superoxide dismutase (SOD) protein operably linked to a promoter flanked by the first AAV inverted terminal repeat (ITR) sequence and the second AAV ITR.

The disclosure further includes a method of treating, ameliorating, and/or preventing progressive hearing loss in a subject need thereof. In certain embodiments, the method comprises administering to the subject an effective amount of an adeno-associated virus (AAV) vector comprising a nucleic acid encoding a superoxide dismutase (SOD) protein operably linked to a promoter. In certain embodiments, the SOD protein is expressed in an inner ear cell. In certain embodiments, the administering treats, ameliorates, and/or prevents the progressive hearing loss in the subject.

The disclosure further includes a method of reversing, minimizing, and/or preventing progressive hearing loss in a subject need thereof. In certain embodiments, the method comprises administering to the subject an effective amount of an adeno-associated virus (AAV) vector comprising a nucleic acid encoding a superoxide dismutase (SOD) protein operably linked to a promoter. In certain embodiments, the SOD protein is expressed in an inner ear cell of the subject. In certain embodiments, the administering reverses, minimizes, and/or prevents the progressive hearing loss in the subject.

The disclosure further includes a method of reversing, minimizing, and/or preventing chemotherapy treatment-associated hearing loss in a subject need thereof. In certain embodiments, the method comprises administering to the subject an effective amount of an adeno-associated virus (AAV) vector comprising a nucleic acid encoding a superoxide dismutase (SOD) protein operably linked to a promoter. In certain embodiments, the SOD protein is expressed in an inner ear cell of the subject. In certain embodiments, the administering reverses, minimizes, and/or prevents the chemotherapy treatment-associated hearing loss in the subject.

### BRIEF DESCRIPTION OF THE DRAWINGS

The following detailed description of selected embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1D illustrate a hypothesized mechanism of SOD1 neuroprotection during noise exposure. FIG. TA is a drawing of the hair cell showing the location of SOD1 (intracellular), SOD2 (mitochondrial), and SOD3 (extracellular). All three are viable candidates for neuroprotection from various forms of oxidative stress. FIG. 1B is a cartoon showing how prolonged noise exposure overwhelm the intracellular SOD1 pathway with reactive oxygen species (ROS). FIG. 1C is a cartoon showing the hypothetical mechanism of delivery of targeted AAV to the hair cell, which induces the up regulation of SOD1 transcription within the cell. FIG. 1D is a diagram of the hypothetical mechanism of neuroprotection from noise in the AAV protected hair cell.

FIGS. 2A-2H illustrate the targeted delivery of AAV to the cochlea through direct round window injections and global cisterna magna injections. FIG. 2A is a drawing of the ear illustrating positioning of the target injection site, the round window (RW) within the cochlea. FIG. 2B is a drawing of the surgical site (Sx) for round window injection, which uses entry through the bulla of the ear. FIG. 2C is a photomicrograph taken with the surgical camera shows the round window through the bulla opening. FIG. 2D illustrates the successful penetration of the round window for injection with AAV. FIG. 2E is a photomicrograph taken with the surgical camera shows the exposed cisterna magna. FIG. 2F is a higher resolution image showing the needle inserted successfully into the cisterna magna for AAV injection. FIG. 2G is a drawing showing one pathway for how injection of AAV through the cisterna magna allows delivery through the cochlear aqueduct, where AAV is expressed in targeted cell populations (e.g., inner hair cell [IHC]/outer hair cell [OHC]). FIG. 2H is a drawing showing a second hypothetical pathway for AAV travel from the cisterna magna through the cerebral spinal fluid (CSF) that runs along the internal auditory canal which holds the auditory nerve.

FIGS. 3A-3E illustrate cisterna magna injection of targeted AAV to express SOD1 protein. FIG. 3A is a brightfield micrograph (2×) showing a horizontal section of the cochlea (5 mM). Lines indicate the ROI hair cell complex, which holds two of the patent target cell types (inner and outer hair cells). Dashed box shows area depicted in B. FIG. 3B is a brightfield micrograph (10×) showing a cross section of the hair cell complex in the higher frequency region of the cochlea. FIG. 3C is a brightfield micrograph (40×) showing the hair cell complex boxed in FIG. 3B. Here the 3 outer hair cells that are connected to the inner hair cell and innervated by the primary afferent dendrites of the auditory nerve ganglion and surrounding support cells can be clearly seen. FIG. 3D is a fluorescence image (FITC, 40×) of the hair cell complex from an animal that received 1 μL of AAV2.7m8.SOD1.GFP two weeks prior. Here a medium level of IHC and OHC GFP expression can be seen suggesting decent expression of the SOD1 enzyme at this dose. FIG. 3E is a fluorescence image (FITC, 40×) of the hair cell complex from an animal that received 2 μL of AAV2.7m8.SOD1.GFP two weeks prior. Here a higher level of IHC and OHC GFP expression can be seen indicating better expression of the SOD1 enzyme at this dose.

FIGS. 4A-4D illustrate that SOD1 expression offers protection from noise-induced hearing loss (increased auditory thresholds). FIG. 4A is a diagram illustrating the auditory brainstem response technique. Drawing (top-left) shows the auditory pathway activated by sound stimuli at various frequencies and how these map onto the signal produced in the 7 peaks of the ABR waves (bottom-left). Drawing (top-right) of the subcutaneous electrode configuration to carry out the ABR and representative examples (bottom-right) of the typical effect of noise-exposure on ABR thresholds (worsened by 10 to 30 dB SPL). FIG. 4B is a scatter plot showing the shifts in ABR threshold in pre-noise and post-noise at each frequency (1, 2, 4, 8, and 16 kHz) for saline control (left) and SOD1 animals (right). Dashed line shows a common threshold where mild to moderate hearing loss is diagnosed in humans (worsened by 20 to 40 dB SPL). FIG. 4C is a graph showing the direct comparison of the pre and post noise threshold difference scores (post−pre) between saline control and SOD1 animals. Arrow illustrates data where there was no worsening of threshold between pre and post noise at that frequency. FIG. 4D is a representative examples of the worsened threshold pre and post noise for saline CTL (left) and SOD1 animals (right). *** p<0.001.

FIGS. 5A-5D illustrate that SOD1 expression offers protection from noise-induced hearing loss changes to ABR amplitude and latency. FIG. 5A is a scatterplot shows the matched comparison of pre-noise amplitudes at each frequency to their post noise amplitudes at each frequency (1, 2, 4, 8, and 16 kHz) for Saline Control (left) and SOD1 animals (right). Dashed line underscores the number of instances where an amplitude was recorded pre-noise followed by no sound induced response post-noise. FIG. 5B is a graph showing the direct comparison of the pre and post noise amplitude difference scores (post−pre) between saline control and SOD1 animals. FIG. 5C is a scatterplot shows the matched comparison of pre-noise latency at each frequency to their post noise latency at each frequency (1, 2, 4, 8, and 16 kHz) for saline control (left) and SOD1 animals (right). Dashed line underscores the number of instances where a latency was recorded pre-noise followed by no sound induced response post-noise. FIG. 5D is a graph showing the direct comparison of the pre and post noise latency difference scores (post−pre) between Saline control and SOD1 animals. *** p<0.001.

FIG. 6 is a map of the AAV2-7m8 vector which was used to target the hair cells.

FIGS. 7A-7B illustrate long term safety and efficacy data for SOD1 neuroprotection against noise exposure. FIG. 7A is a cartoon showing the normal effect of noise induced oxidative stress on a normal cell (left) and a cell with SOD gene therapy (right). Increased bioavailability of SOD provides neuroprotection from free radicals over produced by noise exposure. FIG. 7B is a line graph showing data from a group of animals (N8) that received SOD1 injections (5 μl) followed by 5 months of monitoring for auditory health (left). These animals were then exposed to 110 dB of sound (2 Hrs) for two weeks. Here the SOD1 treatment provided strong neuroprotection even after five months of transgene expression (middle). This suggests that transgene expression is continuous, and that neuroprotection is stable across time. Finally, after another three months of tracking hearing status, animals were exposed to 110 dB noise (2 Hrs) for another two weeks. Again the SOD1 treatment provided continued neuroprotection from noise exposure showing a robust efficacy.

FIGS. 8A-8F illustrate SOD1 neuroprotection against acute traumatic noise exposure. FIG. 8A is a cartoon showing the effect of acoustic trauma (120 dB, 30 minutes) on the cochlear complex of the inner ear. FIG. 8B is a line graph showing data for control animals exposed to 120 dB SPL noise for 30 minutes, 1 hour, and 2 hours. These animals developed significant persistent hearing loss over 4 weeks. Animals that received SOD1 treatment prior to exposure had significant neuroprotection overtime. Animals that received SOD1 treatment after noise exposure (1 hour post) had a significant savings of hearing loss that corresponded to the expression of SOD1 transgene over time (˜2 weeks to peak). FIG. 8C is a scatter plot showing the significant neuroprotection for pretreated animals and those that were treated after noise exposure. FIGS. 8D-8F are line graphs showing data from FIG. 8B broken down by frequency and group.

FIGS. 9A-9C illustrate SOD1 neuroprotection from cisplatin-induced hearing loss over time. FIG. 9A is a line graph showing the gradual buildup of cisplatin (weekly I.P. 2 mg/kg) that leads to hearing loss in the control group and the neuroprotection offered by SOD1 gene therapy. FIG. 9B illustrates data for the control group showing that hearing loss occurs across all frequencies as damage occurs, while FIG. 9C shows that SOD1 pretreatment protected these animals across all frequencies throughout the cisplatin treatment regimen.

FIGS. 10A-10B illustrate SOD1 neuroprotection from cisplatin-induced hearing loss at low, mid, and high frequencies. FIG. 10A is a line graph showing that all frequency ranges are affected by cisplatin in the control group. SOD1 pretreatment produced nearly 100% neuroprotection across frequency ranges. FIG. 10B is a scatterplot showing the significance in the SOD1 gene therapy group over the control group.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, illustrative materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example. “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “AAV vector” as used herein refers to a polynucleotide vector comprising one or more genes of interest (or transgenes) that are flanked by AAV terminal repeat sequences (ITRs). AAV vectors can be produced and packaged into infectious viral particles when present in a host cell that has been transfected with one or more helper plasmids encoding and expressing rep and cap proteins and one or more proteins from adenovirus open reading frame E4 open reading frame 6. The AAV vectors may be operably linked to promoter and enhancer sequences that can regulate the expression of the protein encoded by the AAV vector.

The terms “AAV virion” or “AAV viral particle” or “AAV vector particle” as used herein refers to a viral particle composed of capsid proteins from at least one AAV serotype surrounding a polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

The term “packaging” as used herein refers to intracellular process by which viral virions or particles (e.g., AAV virions or particles), especially viral vector particles or virions are assembled in a host cell. “Packaging” cells comprise the polynucleotide (e.g., helper plasmids) and protein components necessary to assemble functional viral virions.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

By “biologic sample” is meant any tissue, cell, fluid, or other material derived from an organism.

As used herein, the term “cassette” or “expression cassette” or “regulatory cassette” refer to distinct nucleic acid vectors consisting of a payload transgene and regulatory sequences (i.e., promoter, enhancers, terminators, and the like) which control its expression. Upon successful insertion into a host cell, the regulatory sequences allow for the transcription and translation of the payload transgene.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions, e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “epigenetic” as used herein refers to heritable influences on gene expression that do not involve alterations in DNA nucleotide sequence. Epigenetic regulation can enhance or inhibit expression of affected genes and can involve chemical modifications of the deoxyribose backbone of the DNA or the association of DNA/histone protein complexes or both.

The term “epigenetic regulator” as used herein refers to factors, enzymes, compounds, or compositions that act to alter the epigenetic status of a specific DNA locus. Epigenetic regulators can induce or catalyze the modification of DNA-associated proteins or the chemical structure of the DNA itself.

The terms “epigenetic tag” or “epigenetic marker” or “epigenetic mark” as used interchangeably herein, describe the specific chemical modifications made to DNA and DNA-associated proteins that result in epigenetic regulation of gene expression. Examples of epigenetic marks or tags can include but are not limited to the addition or removal of methyl or acetyl groups from CpG dinucleotides and histone proteins. The number and density of epigenetic tags or marks may correlate with the degree of epigenetic regulation a particular DNA locus is subject to.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as non-human primate, ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “knockout mouse” refers to a mouse that has had an existing gene inactivated (i.e., “knocked out”). In some embodiments, the gene is inactivated by homologous recombination. In some embodiments, the gene is inactivated by replacement or disruption with an artificial nucleic acid sequence.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention is based on the unexpected observation that expression of the superoxide dismutase or SOD gene in certain cells of the inner ear, including hair cells, can protect these cells from damage and death caused by the production of reactive oxygen species (ROS) due to stresses including overstimulation. In certain embodiments, the SOD gene is delivered to the cells of the inner ear via a recombinant adeno-associated virus (AAV) vector which efficiently delivers a nucleic acid encoding the SOD gene operably linked to a promoter which drives 'its expression. In certain embodiments, the invention includes methods of treating, ameliorating, and/or preventing progressive hearing loss comprising the expression of the SOD gene in cells of the inner ear (e.g., hair cells). In this way, the expression of the SOD gene acts to protect the cells from damage caused by ROS production.

### Reactive Oxygen Species in Progressive Hearing Loss

Reactive Oxygen Species or “ROS”, as used herein, refers a variety of molecules which are derivatives of molecular oxygen including hydroxyl radicals, superoxide anions, hydrogen peroxide, and singlet oxygen among others. In eukaryotic cells, ROS species are typically generated by the mitochondria as a by-product of normal cellular processes such as the production of adenosine triphosphate (ATP) via respiration. While ROS produced by normal processes in moderate amounts play essential roles in cell signaling and are quickly metabolized or scavenged by endogenous antioxidant mechanisms, abnormally elevated production of ROS due to high metabolic activity or in reaction to various cellular stresses can lead to molecular damage. Due to their unstable nature, ROS species react readily with other molecules and can damage proteins, lipids, and nucleic acids. Accumulation of ROS-related damage can lead to a state of oxidative distress that can trigger cellular death pathways such as apoptosis. The accumulation of ROS above normal levels and subsequent apoptosis induction is a key contributor to several diseases and aging, including a number of hearing loss pathologies.

Within the inner ear lies the spiral cavity of the cochlea, containing the organ of Corti, the core auditory component which produces nerve impulses in response to sound vibrations. The organ of Corti contains two types of sensory hair cells, inner and outer hair cells, which possess mechanosensory organelles called stereocilia. Mechanical displacement of the stereocilia open transduction ion channels, resulting in nerve impulses.

Hearing loss is a reduction in sound sensitivity and is roughly divided into two types: acquired hearing loss and inherited hearing loss. Both types of hearing loss typically involve the loss of inner and/or out hair cells within the inner ear, by dramatically reducing the number of cells capable of converting mechanical vibrations (e.g., sound waves) into nerve impulses. There are many well-known types of acquired hearing loss, including, but not limited to, ototoxic drug-induced hearing loss, age-related hearing loss, trauma-induced hearing loss, inflammatory- and autoimmune-induced hearing loss, and noise-induced hearing loss. The two most common classes of drugs known to cause toxicity in inner ear cells (cochlear and vestibular): aminoglycoside antibiotics and platinum-based cancer chemotherapy drugs (e.g., cisplatin). Both classes induce damage to hair cells by inducing excessive ROS production leading to apoptosis. The death of hair cells due to apoptosis leads to irreversible hearing deficits. Recent studies have identified that aminoglycosides tend to accumulate in the mitochondria of hair cells where they inhibit mitochondrial ribosome function and trigger ion permeability. Platinum-based chemotherapy drugs induce short-term acute effects on transduction and voltage-dependent calcium currents and long-lasting changes to potassium conductance within hair cell mitochondria which trigger ROS production and apoptosis. Age-related hearing loss is typically associated with the accumulation of mutations within mitochondrial DNA encoding components of the oxidative phosphorylation leading to increased ROS production. Noise-induced hearing loss is a common cause of progressive hearing deficits and is often associated with high-noise environments such as the military, concert venues, and industrial activities. Here, mechanical damage to the stereocilia causes the production of ROS and the release of other apoptosis-inducing factors, which lead to the accumulative loss of hair cells.

In certain embodiments, the present disclosure includes compositions and methods which act to reduce or prevent the accumulation of increased ROS via the expression of superoxide dismutase proteins, which absorb and break-down ROS molecules. In certain embodiments, the invention includes vectors, such as viral vectors, capable of inducing the expression of superoxide dismutase proteins in inner ear cells (e.g., inner and outer hair cells). In certain other embodiments, the invention includes vectors, such as viral vectors, capable of inducing the expression of superoxide dismutase proteins in other cochlear cells (e.g., Deiter cells, Hansen cells, Claudius cells, primary afferent neurons, supporting cells, limbus cells, spiral ligament cells, stria vascularis cells) as well as type I and type II vestibular hair cells in the vestibular end-organs. In this way, the expression of superoxide dismutase proteins absorbs the excess ROS molecules and prevents the induction of apoptosis and thus protects against hearing loss associated with the death of inner ear cells, as well as loss of vestibular hair cells due to aging or ototoxicity.

### Superoxide Dismutase Proteins

Superoxide dismutases (SODs) are family of enzymes whose primary function is to catalyze the dismutation or disproportionation of the superoxide anion radicals, a common component of ROS, into more inert molecular oxygen and hydrogen peroxide (which is degraded by other enzymes such as catalase). Three members of the SOD family have been described in mammalian cells. SOD1, or CuZn-SOD was the first to be characterized and is a copper and zinc-containing homodimer that is commonly expressed in intracellular cytoplasmic spaces. SOD2 or Mn-SOD2 is a manganese-containing enzyme which exists as a tetramer and is targeted to the mitochondria. SOD3 or EC-SOD, is a copper and zinc-containing tetramer that is commonly secreted into extracellular environments. Due to their potent antioxidant properties, SOD proteins are widely expressed by both prokaryotic and eukaryotic cells. Loss of function of SOD proteins is associated with a number of genetic diseases, including familial amyotrophic lateral sclerosis, which results from mutations in SOD1 and certain cardiomyopathies.

In certain embodiments, the current disclosure includes compositions and methods for protecting against progressive hearing loss and arresting the progression of hearing loss through the expression of SOD proteins in the cells of the inner ear (e.g., inner and outer hair cells) which act to break-down elevated levels of ROS due to chemical, overstimulation, or other environmental or genetic factors. In addition, in certain embodiments, the current disclosure includes compositions and methods for protecting against progressive peripheral vestibular loss and arresting the progression of vestibular loss through the expression of SOD proteins in the cells of the inner ear vestibular end-organs (e.g., type I and type II vestibular hair cells) which act to break-down elevated levels of ROS due to chemical, overstimulation, or other environmental or genetic factors. In certain embodiments, SOD protein expression is induced by the transduction of inner ear cells with nucleic acids encoding a SOD protein through the use of a recombinant AAV viral vector. In certain embodiments, the SOD protein is a SOD1 protein. In certain embodiments, the SOD protein is a SOD2 protein. In certain embodiments, the SOD protein is a SOD3 protein. It is also contemplated that the methods and compositions of the invention can comprise any combination of SOD1, SOD2, and SOD3 proteins and their variants, such that protection of inner ear cells from oxidative damage is optimized.

### AAV Vectors

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggybacks on co-infection with adenovirus and relies on three key elements: the ITR-flanked genome, the open-reading frames, and adeno-helper genes. Due to their non-pathogenic ability to readily infect human cells, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988: Tratschin et al., 1985: Hermonat and Muzyczka, 1984.

AAV-based vector systems typically separate the viral AAV genes, Adenovirus-derived helper genes, and the transgene payload onto two or three separate plasmids. Three plasmid systems consist of an AAV helper plasmid comprising the rep (replication) and cap (capsid) genes, an adenoviral helper plasmid comprising at least the E2a gene, E4 gene, and VA (viral associated) RNA, and a payload plasmid comprising the transgene and associated promoters and enhancers flanked by ITR sequences. The helper plasmid or plasmids do not comprise ITRs in order to prevent packaging of a functional, infectious viral genome.

Two plasmid systems combine the AAV rep and cap genes and adenoviral helper genes onto a single plasmid and simplify viral vector production by reducing the number of transfected plasmids. Often, a dedicated packaging cell line is used which is engineered to express AAV/helper genes prior to introduction of the payload plasmid.

Successful gene therapies require efficient infection of target tissues and establishment of long-term gene expression, and AAV vectors can successfully infect and transduce a broad variety of cell and tissue types, such as brain, liver, and muscle, among others, and have the ability to infect both dividing and quiescent cells. Additionally. AAV-mediated transduction of tissues has been demonstrated to result in long term transgene expression greater than 1.5 years in animal models including canine, murine and hamster.

The tissue tropism of AAV vector particles is influenced by the serotype of the capsid protein, though the receptors and co-receptors that the capsid proteins bind to are often poorly understood and can be expressed by multiple tissue types. For example, AAV2, one of the most well-studied serotypes, has a binding affinity largely for heparan sulfate proteoglycan (HSPG) and as such has a tropism in humans for eye, brain, lung, liver, muscle, and joint tissues. Likewise, AAVs 1, 4, 5, and 6 have a binding affinity largely for sialic acid and a tropism for neuronal tissues while AAVs 5 and 8 share a tropism for skeletal muscle. In this way, the serotype of the AAV capsid protein can be selected to target the payload nucleic acid (e.g., a regulatory cassette) of the AAV vector to a specific tissue or cell type. Alteration or modification of capsid protein structure can also alter the tissue or cellular tropism and affinity of the resulting AAV vector particles.

In certain embodiments, the current disclosure comprises AAV vectors comprising a capsid protein derived from AAV9 and variants thereof. AAV9 and capsid proteins based on AAV9 transduce muscle, liver, and lung tissues about 100-fold more efficiently than AAV2, while also being able to cross the blood brain barrier.

In certain embodiments, the current disclosure comprises AAV vectors comprising a capsid protein derived from AAV2. One nonlimiting example of a derivative of an AAV2 capsid protein is AAV2-7m8. AAV2-7m8 contains a 10-amino acid peptide inserted at position 588 of the AAV2 capsid protein sequence, which is involved with AAV2 binding to its primary receptor, heparan sulfate proteoglycan and was originally characterized as having increased infection efficiency of mouse photoreceptor cells. AAV2-7m8 has the ability to infect both inner and outer hair cells of the cochlea as well as the type I and type II vestibular hair cells of the vestibular end-organs with high efficiency.

It is contemplated that the SOD protein-expressing AAV vectors and compositions of the disclosure can be used with any naturally occurring, modified, hybrid, or engineered AAV capsid protein that provides the desired tissue tropism (e.g., inner and outer hair cells of the inner ear, supporting cells, stria vascularis cells, other cochlear cells, as well as type I and type II vestibular hair cells of the vestibular end-organs) including, but not limited to AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark100 among others. The skilled artisan would be able to select an appropriate capsid protein for use with the disclosure based on the desired target tissue or cell type.

AAV2-7m8
(SEQ ID NO: 4)
GAATTCCCATCATCAATAATATACCTTATTTTGGATTGAAGCCAATATGA
TAATGAGGGGGTGGAGTTTGTGACGTGGCGCGGGGCGTGGGAACGGGGGG
GGTGACGTAGTAGCTCTAGAGGTCCTGTATTAGAGGTCACGTGAGTGTTT
TGCGACATTTTGCGACACCATGTGGTCACGCTGGGTATTTAAGCCCGAGT
GAGCACGCAGGGTCTCCATTTTGAAGCGGGAGGTTTGAACGCGCAGCCAC
CACGGCGGGGTTTTACGAGATTGTGATTAAGGTCCCCAGCGACCTTGACG
AGCATCTGCCCGGCATTTCTGACAGCTTTGTGAACTGGGTGGCCGAGAAG
GAATGGGAGTTGCCGCCAGATTCTGACATGGATCTGAATCTGATTGAGCA
GGCACCCCTGACCGTGGCCGAGAAGCTGCAGCGCGACTTTCTGACGGAAT
GGCGCCGTGTGAGTAAGGCCCCGGAGGCCCTTTTCTTTGTGCAATTTGAG
AAGGGAGAGAGCTACTTCCACATGCACGTGCTCGTGGAAACCACCGGGGT
GAAATCCATGGTTTTGGGACGTTTCCTGAGTCAGATTCGCGAAAAACTGA
TTCAGAGAATTTACCGCGGGATCGAGCCGACTTTGCCAAACTGGTTCGCG
GTCACAAAGACCAGAAATGGCGCCGGAGGCGGGAACAAGGTGGTGGATGA
GTGCTACATCCCCAATTACTTGCTCCCCAAAACCCAGCCTGAGCTCCAGT
GGGCGTGGACTAATATGGAACAGTATTTAAGCGCCTGTTTGAATCTCACG
GAGCGTAAACGGTTGGTGGCGCAGCATCTGACGCACGTGTCGCAGACGCA
GGAGCAGAACAAAGAGAATCAGAATCCCAATTCTGATGCGCCGGTGATCA
GATCAAAAACTTCAGCCAGGTACATGGAGCTGGTCGGGTGGCTCGTGGAC
AAGGGGATTACCTCGGAGAAGCAGTGGATCCAGGAGGACCAGGCCTCATA
CATCTCCTTCAATGCGGCCTCCAACTCGCGGTCCCAAATCAAGGCTGCCT
TGGACAATGCGGGAAAGATTATGAGCCTGACTAAAACCGCCCCCGACTAC
CTGGTGGGCCAGCAGCCCGTGGAGGACATTTCCAGCAATCGGATTTATAA
AATTTTGGAACTAAACGGGTACGATCCCCAATATGCGGCTTCCGTCTTTC
TGGGATGGGCCACGAAAAAGTTCGGCAAGAGGAACACCATCTGGCTGTTT
GGGCCTGCAACTACCGGGAAGACCAACATCGCGGAGGCCATAGCCCACAC
TGTGCCCTTCTACGGGTGCGTAAACTGGACCAATGAGAACTTTCCCTTCA
ACGACTGTGTCGACAAGATGGTGATCTGGTGGGAGGAGGGGAAGATGACC
GCCAAGGTCGTGGAGTCGGCCAAAGCCATTCTCGGAGGAAGCAAGGTGCG
CGTGGACCAGAAATGCAAGTCCTCGGCCCAGATAGACCCGACTCCCGTGA
TCGTCACCTCCAACACCAACATGTGCGCCGTGATTGACGGGAACTCAACG
ACCTTCGAACACCAGCAGCCGTTGCAAGACCGGATGTTCAAATTTGAACT
CACCCGCCGTCTGGATCATGACTTTGGGAAGGTCACCAAGCAGGAAGTCA
AAGACTTTTTCCGGTGGGCAAAGGATCACGTGGTTGAGGTGGAGCATGAA
TTCTACGTCAAAAAGGGTGGAGCCAAGAAAAGACCCGCCCCCAGTGACGC
AGATATAAGTGAGCCCAAACGGGTGCGCGAGTCAGTTGCGCAGCCATCGA
CGTCAGACGCGGAAGCTTCGATCAACTACGCAGACAGGTACCAAAACAAA
TGTTCTCGTCACGTGGGCATGAATCTGATGCTGTTTCCCTGCAGACAATG
CGTGAGAATGAATCAGAATTCAAATATCTGCTTCACTCACGGACAGAAAG
ACTGTTTAGAGTGCTTTCCCGTGTCAGAATCTCAACCCGTTTCTGTCGTC
AAAAAGGCGTATCAGAAACTGTGCTACATTCATCATATCATGGGAAAGGT
GCCAGACGCTTGCACTGCCTGCGATCTGGTCAATGTGGATTTGGATGACT
GCATCTTTGAACAATAAATGATTTAAATCAGGTATGGCTGCCGATGGTTA
TCTTCCAGATTGGCTCGAGGACACTCTCTCTGAAGGAATAAGACAGTGGT
GGAAGCTCAAACCTGGCCCACCACCACCAAAGCCCGCAGAGCGGCATAAG
GACGACAGCAGGGGTCTTGTGCTTCCTGGGTACAAGTACCTCGGACCCTT
CAACGGACTCGACAAGGGAGAGCCGGTCAACGAGGCAGACGCCGCGGCCC
TCGAGCACGACAAAGCCTATGACCGGCAGCTCGACAGCGGAGACAACCCG
TACCTCAAGTACAACCACGCCGACGCGGAGTTTCAGGAGCGCCTTAAAGA
AGATACGTCTTTTGGGGGCAACCTCGGACGAGCAGTCTTCCAGGCGAAAA
AGAGGGTTCTTGAACCTCTGGGCCTGGTTGAGGAACCTGTTAAGACGGCT
CCGGGAAAAAAGAGGCCGGTAGAGCACTCTCCTGTGGAGCCAGACTCCTC
CTCGGGAACCGGAAAGGCGGGCCAGCAGCCTGCAAGAAAAAGATTGAATT
TTGGTCAGACTGGAGACGCAGACTCAGTACCTGACCCCCAGCCTCTCGGA
CAGCCACCAGCAGCCCCCTCTGGTCTGGGAACTAATACGATGGCTACAGG
CAGTGGCGCACCAATGGCAGACAATAACGAGGGCGCCGACGGAGTGGGTA
ATTCCTCGGGAAATTGGCATTGCGATTCCACATGGATGGGCGACAGAGTC
ACCACCACCAGCACCCGAACCTGGGCCCTGCCCACCTACAACAACCACCT
CTACAAACAAATTTCCAGCCAATCAGGAGCCTCGAACGACAATCACTACT
TTGGCTACAGCACCCCTTGGGGGTATTTTGACTTCAACAGATTCCACTGC
CACTTTTCACCACGTGACTGGCAAAGACTCATCAACAACAACTGGGGATT
CCGACCCAAGAGACTCAACTTCAAGCTCTTTAACATTCAAGTCAAAGAGG
TCACGCAGAATGACGGTACGACGACGATTGCCAATAACCTTACCAGCACG
GTTCAGGTGTTTACTGACTCGGAGTACCAGCTCCCGTACGTCCTCGGCTC
GGCGCATCAAGGATGCCTCCCGCCGTTCCCAGCAGACGTCTTCATGGTGC
CACAGTATGGATACCTCACCCTGAACAACGGGAGTCAGGCAGTAGGACGC
TCTTCATTTTACTGCCTGGAGTACTTTCCTTCTCAGATGCTGCGTACCGG
AAACAACTTTACCTTCAGCTACACTTTTGAGGACGTTCCTTTCCACAGCA
GCTACGCTCACAGCCAGAGTCTGGACCGTCTCATGAATCCTCTCATCGAC
CAGTACCTGTATTACTTGAGCAGAACAAACACTCCAAGTGGAACCACCAC
GCAGTCAAGGCTTCAGTTTTCTCAGGCCGGAGCGAGTGACATTCGGGACC
AGTCTAGGAACTGGCTTCCTGGACCCTGTTACCGCCAGCAGCGAGTATCA
AAGACATCTGCGGATAACAACAACAGTGAATACTCGTGGACTGGAGCTAC
CAAGTACCACCTCAATGGCAGAGACTCTCTGGTGAATCCGGGCCCGGCCA
TGGCAAGCCACAAGGACGATGAAGAAAAGTTTTTTCCTCAGAGCGGGGTT
CTCATCTTTGGGAAGCAAGGCTCAGAGAAAACAAATGTGGACATTGAAAA
GGTCATGATTACAGACGAAGAGGAAATCAGGACAACCAATCCCGTGGCTA
CGGAGCAGTATGGTTCTGTATCTACCAACCTCCAGAGAGGCAACCTAGCA
CTCGGCGAAACAACAAGACCTGCTAGGCAAGCAGCTACCGCAGATGTCAA
CACACAAGGCGTTCTTCCAGGCATGGTCTGGCAGGACAGAGATGTGTACC
TTCAGGGGCCCATCTGGGCAAAGATTCCACACACGGACGGACATTTTCAC
CCCTCTCCCCTCATGGGTGGATTCGGACTTAAACACCCTCCTCCCCAGAT
TCTCATCAAGAACACCCCGGTACCTGCGAATCCTTCGACCACCTTCAGTG
CGGCAAAGTTTGCTTCCTTCATCACACAGTACTCCACGGGACAGGTCAGC
GTGGAGATCGAGTGGGAGCTGCAGAAGGAAAACAGCAAACGCTGGAATCC
CGAAATTCAGTACACTTCCAACTACAACAAGTCTATTAATGTGGACTTTA
CTGTGGACACTAATGGCGTGTATTCAGAGCCTCGCCCCATTGGCACCAGA
TACCTGACTCGTAATCTGTAATTGCGGCCGCTTGTTAATCAATAAACCGT
TTAATTCGTTTCAGTTGAACTTTGGTCTCTGCGTATTTCTTTCTTATCTA
GTTTCCATGCTCTAGAGGTCCTGTATTAGAGGTCACGTGAGTGTTTTGCG
ACATTTTGCGACACCATGTGGTCACGCTGGGTATTTAAGCCCGAGTGAGC
ACGCAGGGTCTCCATTTTGAAGCGGGAGGTTTGAACGCGCAGCCACCACG
GCGGGGTTTTACGAGATTGTGATTAAGGTCCCCAGCGACCTTGACGAGCA
TCTGCCCGGCATTTCTGACAGCTTTGTGAACTGGGTGGCCGAGAAGGAAT
GGGAGTTGCCGCCAGATTCTGACATGGATCTGAATCTGATTGAGCAGGCA
CCCCTGACCGTGGCCGAGAAGCTGCATCGCTGGCGTAATAGCGAAGAGGC
CCGCACCGATCGCCCTTCCCAACAGTTGCGCAGCCTGAATGGCGAATGGC
GATTCCGTTGCAATGGCTGGCGGTAATATTGTTCTGGATATTACCAGCAA
GGCCGATAGTTTGAGTTCTTCTACTCAGGCAAGTGATGTTATTACTAATC
AAAGAAGTATTGCGACAACGGTTAATTTGCGTGATGGACAGACTCTTTTA
CTCGGTGGCCTCACTGATTATAAAAACACTTCTCAGGATTCTGGCGTACC
GTTCCTGTCTAAAATCCCTTTAATCGGCCTCCTGTTTAGCTCCCGCTCTG
ATTCTAACGAGGAAAGCACGTTATACGTGCTCGTCAAAGCAACCATAGTA
CGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTC
TTCCCTTCCTTTCTCGCCACGTTCGCCGGCTTTCCCCGTCAAGCTCTAAA
TCGGGGGCTCCCTTTAGGGTTCCGATTTAGTGCTTTACGGCACCTCGACC
CCAAAAAACTTGATTAGGGTGATGGTTCACGTAGTGGGCCATCGCCCTGA
TAGACGGTTTTTCGCCCTTTGACGTTGGAGTCCACGTTCTTTAATAGTGG
ACTCTTGTTCCAAACTGGAACAACACTCAACCCTATCTCGGTCTATTCTT
TTGATTTATAAGGGATTTTGCCGATTTCGGCATATTGGTTAAAAAATGAG
CTGATTTAACAAAAATTTAACGCGAATTTTAACAAAATATTAACGCTTAC
AATTTAAATATTTGCTTATACAATCTTCCTGTTTTTGGGGCTTTTCTGAT
TATCAACCGGGGTACATATGATTGACATGCTAGTTTTACGATTACCGTTC
ATCGATTCTCTTGTTTGCTCCAGACTCTCAGGCAATGACCTGATAGCCTT
TGTAGAGACCTCTCAAAAATAGCTACCCTCTCCGGCATGAATTTATCAGC
TAGAACGGTTGAATATCATATTGATGGTGATTTGACTGTCTCCGGCCTTT
CTCACCCGTTTGAATCTTTACCTACACATTACTCAGGCATTGCATTTAAA
ATATATGAGGGTTCTAAAAATTTTTATCCTTGCGTTGAAATAAAGGCTTC
TCCCGCAAAAGTATTACAGGGTCATAATGTTTTTGGTACAACCGATTTAG
CTTTATGCTCTGAGGCTTTATTGCTTAATTTTGCTAATTCTTTGCCTTGC
CTGTATGATTTATTGGATGTTGGAATCGCCTGATGCGGTATTTTCTCCTT
ACGCATCTGTGCGGTATTTCACACCGCATATGGTGCACTCTCAGTACAAT
CTGCTCTGATGCCGCATAGTTAAGCCAGCCCCGACACCCGCCAACACCCG
CTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATC
ACCGAAACGCGCGAGACGAAAGGGCCTCGTGATACGCCTATTTTTATAGG
TTAATGTCATGATAATAATGGTTTCTTAGACGTCAGGTGGCACTTTTCGG
GGAAATGTGCGCGGAACCCCTATTTGTTTATTTTTCTAAATACATTCAAA
TATGTATCCGCTCATGAGACAATAACCCTGATAAATGCTTCAATAATATT
GAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATT
ATCCCGTATTGACGCCGGGCAAGAGCAACTCGGTCGCCGCATACACTATT
CTCAGAATGACTTGGTTGAGTACTCACCAGTCACAGAAAAGCATCTTACG
GATGGCATGACAGTAAGAGAATTATGCAGTGCTGCCATAACCATGAGTGA
TAACACTGCGGCCAACTTACTTCTGACAACGATCGGAGGACCGAAGGAGC
TAACCGCTTTTTTGCACAACATGGGGGATCATGTAACTCGCCTTGATCGT
TGGGAACCGGAGCTGAATGAAGCCATACCAAACGACGAGCGTGACACCAC
GATGCCTGTAGCAATGGCAACAACGTTGCGCAAACTATTAACTGGCGAAC
TACTTACTCTAGCTTCCCGGCAACAATTAATAGACTGGATGGAGGCGGAT
AAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAG
CACTGGGGCCAGATGGTAAGCCCTCCCGTATCGTAGTTATCTACACGACG
GGGAGTCAGGCAACTATGGATGAACGAAATAGACAGATCGCTGAGATAGG
TGCCTCACTGATTAAGCATTGGTAACTGTCAGACCAAGTTTACTCATATA
TACTTTAGATTGATTTAAAACTTCATTTTTAATTTAAAAGGATCTAGGTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTTCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTG
AGCTGATACCGCTCGCCGCAGCCGAACGACCGAGCGCAGCGAGTCAGTGA
GCGAGGAAGCGGAAGAGCGCCCAATACGCAAACCGCCTCTCCCCGCGCGT
TGGCCGATTCATTAATGCA
CAG
(SEQ ID NO: 5)
GCGTTACATAACTTACGGTAAATGGCCCGCCTGGCTGACCGCCCAACGAC
CCCCGCCCATTGACGTCAATAATGACGTATCGCAATGTATTGAATGCCAT
TTACCGGGCGGACCGACTGGCGGGTTGCTGGGGGGGGTAACTGCAGTTAT
TACTGCATAGTTCCCATAGTAACGCCAATAGGGACTTTCCATTGACGTCA
ATGGGTGGAGTATTTACGGTAAACTGCCCACTTGGCAGTCAAGGGTATCA
TTGCGGTTATCCCTGAAAGGTAACTGCAGTTACCCACCTCATAAATGCCA
TTTGACGGGTGAACCGTCAACATCAAGTGTATCATATGCCAAGTACGCCC
CCTATTGACGTCAATGACGGTAAATGGCCCGCCTGGCATTATGCCCAGTT
GTAGTTCACATAGTATACGGTTCATGCGGGGGATAACTGCAGTTACTGCC
ATTTACCGGGCGGACCGTAATACGGGTCAACATGACCTTATGGGACTTTC
CTACTTGGCAGTACATCTACGTATTAGTCATCGCTATTACCATGGTCGAG
GTGAGCCCCTGTACTGGAATACCCTGAAAGGATGAACCGTCATGTAGATG
CATAATCAGTAGCGATAATGGTACCAGCTCCACTCGGGGACGTTCTGCTT
CACTCTCCCCATCTCCCCCCCCTCCCCACCCCCAATTTTGTATTTATTTA
TTTTTTAATTATTTTGTGCTGCAAGACGAAGTGAGAGGGGTAGAGGGGGG
GGAGGGGTGGGGGTTAAAACATAAATAAATAAAAAATTAATAAAACACGA
GCGATGGGGGGGGGGGGGGGGGGGGGGCGCGCGCCAGGCGGGGGGGGGGG
GGGCGAGGGGGGGGGGGGGGCGAGGCGGATCGCTACCCCCGCCCCCCCCC
CCCCCCCGCGCGCGGTCCGCCCCGCCCCGCCCCGCTCCCCGCCCCGCCCC
GCTCCGCCTGAGGTGCGGCGGCAGCCAATCAGAGCGGCGCGCTCCGAAAG
TTTCCTTTTATGGCGAGGCGGCGGCGGCGGCGGCCCTATCTCCACGCCGC
CGTCGGTTAGTCTCGCCGCGCGAGGCTTTCAAAGGAAAATACCGCTCCGC
CGCCGCCGCCGCCGGGATA
Meriones unguiculatus superoxide dismutase 1
(SOD1), mRNA
(SEQ ID NO: 6)
CCGCGGCGCCGGGGTCTCGGTTTGCGCCGGCGTCTCCTGCGGCGGCTTGT
GGCGTCGCTTGCTCTCCCAGCGCTCCCTCAGGTTCCCGAGGCCGTCGCGC
GTGTCCCGGCGAAGCATGGCGATGAAGGCCGTGTGCGTGCTGAAGGGCGA
CGGTCCGGTGCAGGGCACCATCCGCTTCGAGCAGAAGGCAAGCGGTGAAC
CAGTTGTGGTGTCAGGAGAAATTACAGGATTGACTGAAGGCCAGCATGGG
TTCCATGTCCATCAGTTTGGGGATAATACACAAGGGTGTACCAGTGCAGG
ACCTCATTTTAATCCTCATTCCAAGAAACATGGCGGACCAGCAGATCAAG
AGAGGCATGTTGGAGACCTGGGCAATGTGATTGCTGGAAAGGATGGTGTG
GCCAAAGTGTCCATTGAAGATCGTGAGATCTCACTCACAGGAGAGCATTC
CATCATTGGCCGTACAATGGTGGTCCATGAGAAACAAGATGACTTGGGCA
AAGGTGGAAATGATGAAAGTACAAAGACTGGAAATGCTGGAAGCCGCTTG
GCTTGTGGTGTGATTGGGATTGCCCAGTAAACAAACATTCCCTCTGTGGT
CTGAGTCTCAAACTCATCTGCTGTCCTGCTAAGCTGTAGGAAAAAATAAA
CATTAAACTAATCTTTAACAGTGTAACTGTGTGACTCCTTTT
eGFP
(SEQ ID NO: 7)
GACACGTGCTGAAGTCAAGTTTGAAGGTGATACCCTTGTTAATAGAATCG
AGTTAAAAGGTATTGATTTTAAAGAAGATGCTGTGCACGACTTCAGTTCA
AACTTCCACTATGGGAACAATTATCTTAGCTCAATTTTCCATAACTAAAA
TTTCTTCTACGAAACATTCTTGGACACAAATTGGAATACAACTATAACTC
ACACAATGTATACATCATGGCAGACAAACAAAAGAATGGACTTTGTAAGA
ACCTGTGTTTAACCTTATGTTGATATTGAGTGTGTTACATATGTAGTACC
GTCTGTTTGTTTTCTTACCTATCAAAGTTAACTTCAAAATTAGACACAAC
ATTGAAGATGGAAGCGTTCAACTAGCAGACCATTATCAACAAAATACTCC
TAGTTTCAATTGAAGTTTTAATCTGTGTTGTAACTTCTACCTTCGCAAGT
TGATCGTCTGGTAATAGTTGTTTTATGAGGAATTGGCGATGGCCCTGTCC
TTTTACCAGACAACCATTACCTGTCCACACAATCTGCCCTTTCGAAAGAT
CCCAACGAAATTAACCGCTACCGGGACAGGAAAATGGTCTGTTGGTAATG
GACAGGTGTGTTAGACGGGAAAGCTTTCTAGGGTTGCTTTAGAGAGACCA
CATGGTCCTTCTTGAGTTTGTAACAGCTGCTGGGATTACACATGGCATGG
ATGAACTATACAAATAGTCTCTCTGGTGTACCAGGAAGAACTCAAACATT
GTCGACGACCCTAATGTGTACCGTACCTACTTGATATGTTTATC
Woodchuck Hepatitis Virus (WHV) Posttranscriptional
Regulatory Element (WPRE)
(SEQ ID NO: 8)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG
SV40 promoter
(SEQ ID NO: 9)
GGTGTGGAAAGTCCCCAGGCTCCCCAGCAGGCAGAAGTATGCAAAGCATG
CATCTCAATTAGTCAGCAACCAGGTGTGGACCACACCTTTCAGGGGTCCG
AGGGGTCGTCCGTCTTCATACGTTTCGTACGTAGAGTTAATCAGTCGTTG
GTCCACACCTAAGTCCCCAGGCTCCCCAGCAGGCAGAAGTATGCAAAGCA
TGCATCTCAATTAGTCAGCAACCATAGTCCCGCCCCTAACTTCAGGGGTC
CGAGGGGTCGTCCGTCTTCATACGTTTCGTACGTAGAGTTAATCAGTCGT
TGGTATCAGGGCGGGGATTGTCCGCCCATCCCGCCCCTAACTCCGCCCAG
TTCCGCCCATTCTCCGCCCCATGGCTGACTAATTTTTTTTATTTATGCAG
AGGGGGTAGGGCGGGGATTGAGGCGGGTCAAGGCGGGTAAGAGGGGGGTA
CCGACTGATTAAAAAAAATAAATACGTCAGGCCGAGGCCGCCTCGGCCTC
TGAGCTATTCCAGAAGTAGTGAGGAGGCTTTTTTGGAGGCCTAGGCTTTT
GCAAATCCGGCTCCGGCGGAGCCGGAGACTCGATAAGGTCTTCATCACTC
CTCCGAAAAAACCTCCGGATC

### Nucleic Acids and Vectors

The present disclosure provides a polynucleic acid encoding an AAV vector. In certain embodiments, the AAV vector comprises a nucleic acid encoding a SOD1 protein (alternatively known as ALS and ALS1). Examples of sequences encoding SOD1 protein include, but are not limited to NCBI gene. 6647; HGNC, 11179, OMIM, 147450, RefSeq, NM_000454, and UniProt, P00441. In certain embodiments, the nucleic acid encoding the SOD1 protein comprises the Homo sapiens chromosome 21, GRCh38.p14 Primary Assembly NCBI Reference Sequence: NC_000021.9>NC_000021.9:31659693-31668931. In certain embodiments, the nucleic acid encoding a SOD1 protein is derived from Meriones unguiculatus. In certain embodiments, the SOD1 protein is encoded by a nucleic acid comprising a polynucleotide sequence having at least 80%, 85%, 90%, 95%, 96%, 97%, 98%, or 99% identity to SEQ ID NO: 1 or SEQ ID NO: 6. In certain embodiments, the SOD1 protein is encoded by a nucleic acid comprising the nucleotide sequence set forth in SEQ ID NO: 1 or SEQ ID NO: 6.

SOD1 DNA (NM_000454)
(SEQ ID NO: 1)
GCGTCGTAGTCTCCTGCAGCGTCTGGGGTTTCCGTTGCAGTCCTCGGAAC
CAGGACCTCGGCGTGGCCTAGCGAGTTATGGCGACGAAGGCCGTGTGCGT
GCTGAAGGGCGACGGCCCAGTGCAGGGCATCATCAATTTCGAGCAGAAGG
AAAGTAATGGACCAGTGAAGGTGTGGGGAAGCATTAAAGGACTGACTGAA
GGCCTGCATGGATTCCATGTTCATGAGTTTGGAGATAATACAGCAGGCTG
TACCAGTGCAGGTCCTCACTTTAATCCTCTATCCAGAAAACACGGTGGGC
CAAAGGATGAAGAGAGGCATGTTGGAGACTTGGGCAATGTGACTGCTGAC
AAAGATGGTGTGGCCGATGTGTCTATTGAAGATTCTGTGATCTCACTCTC
AGGAGACCATTGCATCATTGGCCGCACACTGGTGGTCCATGAAAAAGCAG
ATGACTTGGGCAAAGGTGGAAATGAAGAAAGTACAAAGACAGGAAACGCT
GGAAGTCGTTTGGCTTGTGGTGTAATTGGGATCGCCCAATAAACATTCCC
TTGGATGTAGTCTGAGGCCCCTTAACTCATCTGTTATCCTGCTAGCTGTA
GAAATGTATCCTGATAAACATTAAACACTGTAATCTTAAAAGTGTAATTG
TGTGACTTTTTCAGAGTTGCTTTAAAGTACCTGTAGTGAGAAACTGATTT
ATGATCACTTGGAAGATTTGTATAGTTTTATAAAACTCAGTTAAAATGTC
TGTTTCAATGACCTGTATTTTGCCAGACTTAAATCACAGATGGGTATTAA
ACTTGTCAGAATTTCTTTGTCATTCAAGCCTGTGAATAAAAACCCTGTAT
GGCACTTATTATGAGGCTATTAAAAGAATCCAAATTCAAACTAAA

In certain embodiments, the invention also provides an AAV vector comprising a nucleic acid encoding a SOD2 protein (alternatively known as Mn-SOD, IPO-B, and MVCD) or isoform or fragment thereof. Examples of sequences encoding SOD2 protein or isoforms or fragments thereof include, but are not limited to NCBI gene, 6648, HGNC, 11180, OMIM, 147460, RefSeq, NM_000636. UniProt, P04179. In certain embodiments, the nucleic acid encoding the SOD2 protein or isoforms or fragment thereof comprises the Homo sapiens chromosome 6, GRCh38.p14 Primary Assembly NCBI Reference Sequence: NC_000006.12>NC_000021.9: 31659693-31668931. In certain embodiments, the SOD2 protein is encoded by a nucleic acid comprising a polynucleotide sequence having at least 80%, 85%, 90%, 95%, 96%, 97%, 98%, or 99% identity to SEQ ID NO: 2. In certain embodiments, the SOD2 protein is encoded by a nucleic acid comprising the nucleotide sequence set forth in SEQ ID NO: 2. In certain embodiments, the SOD2 protein is an isoform or fragment of SOD2 protein that retains some or all of the functions of the full-length SOD2 protein. Non-limiting examples of SOD2 isoforms or fragments that would be encompassed by the invention include sequences encoding SOD2 proteins which have truncations at either the N- or C-terminus. In certain embodiments, these truncations are made in order to reduce the overall size of the sequence encoding the SOD2 protein for example to enable packaging into size-limited vectors (e.g. AAV vectors).

SOD2 DNA (NM_000636)
(SEQ ID NO: 2)
ACTCGTGGCTGTGGTGGCTTCGGCAGCGGCTTCAGCAGATCGGCGGCATC
AGCGGTAGCACCAGCACTAGCAGCATGTTGAGCCGGGCAGTGTGCGGCAC
CAGCAGGCAGCTGGCTCCGGTTTTGGGGTATCTGGGCTCCAGGCAGAAGC
ACAGCCTCCCCGACCTGCCCTACGACTACGGCGCCCTGGAACCTCACATC
AACGCGCAGATCATGCAGCTGCACCACAGCAAGCACCACGCGGCCTACGT
GAACAACCTGAACGTCACCGAGGAGAAGTACCAGGAGGCGTTGGCCAAGG
GAGATGTTACAGCCCAGATAGCTCTTCAGCCTGCACTGAAGTTCAATGGT
GGTGGTCATATCAATCATAGCATTTTCTGGACAAACCTCAGCCCTAACGG
TGGTGGAGAACCCAAAGGGGAGTTGCTGGAAGCCATCAAACGTGACTTTG
GTTCCTTTGACAAGTTTAAGGAGAAGCTGACGGCTGCATCTGTTGGTGTC
CAAGGCTCAGGTTGGGGTTGGCTTGGTTTCAATAAGGAACGGGGACACTT
ACAAATTGCTGCTTGTCCAAATCAGGATCCACTGCAAGGAACAACAGGCC
TTATTCCACTGCTGGGGATTGATGTGTGGGAGCACGCTTACTACCTTCAG
TATAAAAATGTCAGGCCTGATTATCTAAAAGCTATTTGGAATGTAATCAA
CTGGGAGAATGTAACTGAAAGATACATGGCTTGCAAAAAGTAAACCACGA
TCGTTATGCTGAGTATGTTAAGCTCTTTATGACTGTTTTTGTAGTGGTAT
AGAGTACTGCAGAATACAGTAAGCTGCTCTATTGTAGCATTTCTTGATGT
TGCTTAGTCACTTATTTCATAAACAACTTAATGTTCTGAATAATTTCTTA
CTAAACATTTTGTTATTGGGCAAGTGATTGAAAATAGTAAATGCTTTGTG
TGATTGAATCTGATTGGACATTTTCTTCAGAGAGCTAAATTACAATTGTC
ATTTATAAAACCATCAAAAATATTCCATCCATATACTTTGGGGACTTGTA
GGGATGCCTTTCTAGTCCTATTCTATTGCAGTTATAGAAAATCTAGTCTT
TTGCCCCAGTTACTTAAAAATAAAATATTAACACTTTCCCAAGGGAAACA
CTCGGCTTTCTATAGAAAATTGCACTTTTTGTCGAGTAATCCTCTGCAGT
GATACTTCTGGTAGATGTCACCCAGTGGTTTTTGTTAGGTCAAATGTTCC
TGTATAGTTTTTGCAAATAGAGCTGTATACTGTTTAAATGTAGCAGGTGA
ACTGAACTGGGGTTTGCTCACCTGCACAGTAAAGGCAAACTTCAACAGCA
AAACTGCAAAAAGGTGGTTTTTGCAGTAGGAGAAAGGAGGATGTTTATTT
GCAGGGCGCCAAGCAAGGAGAATTGGGCAGCTCATGCTTGAGACCCAATC
TCCATGATGACCTACAAGCTAGAGTATTTAAAGGCAGTGGTAAATTTCAG
GAAAGCAGAAGTTAAAGGCAAAATTGTAAATCAGTCGAGATCGGGTGCCT
TCAGGGTGGTATGGCTGTATACCAAAATTGTAAATCACTACATGAAGCTT
ATATATTGGTTTGGCCTGAAAGGTGAAGTGGGGTAGGCAGGGGGCGGGCT
TACAGGTTATGGTGGATTCAAAGACTCCCTGATTTGTGATTGGTTAAGGA
AGCAAAGCTTTGTCTAAAAACTTGGGGTCCGCAGAAAGGAACATTAAGGT
CTGGCCAGGCCCCTCAGGAAGAAACTGAGAGCAAAGAATGGAGGTCAGAG
TTTAGTCCCTGGTGTTCCCCCTTATCTGACGTCTGTGTGAATCCATTTGG
TGGGGGTCTGGGTTTCTGAAAAGTAGCTCAGGGGCACGTGTTAAGGATGT
CTCTAGGTGACTCTAACTTCCCTGGCTATTGTTTGAAACTGTTATGACCT
TCTTGCTTATCAGCTTGCTGGTTTCCTTCTCGGGGCGAGCTGGGTGCCTG
GAGTTTTCGGTGAAGGAAACTCAAGATTCTCCTTTATTTCTGTGCTTGTG
GGAATCCCCCTGGCACACCCCAAAGAGGGGTCCCTGCTCCGTCTCACAGG
GATCTTTTTGTATATTTGGCTTAGCATCATACATTTGCCATGTTGTTTCA
TCATCTGCCTAATTTACTGTTTTTGAATATTTCATTTGTTTCTAATTGTT
ACTACAGATAATGCTGGGGTGAGCAACTCTGTGTACATAGGTTTATCTCC
TATTGGAATATTTTCTTTATATAGGCGTTTTTTTTTTTTCTTTTTTTTTG
GAGACAGAGTCTTGCTCTGTTGCCCAGGCTGGAGTGCAGTGGCGCGACCG
GAGCTCACTGCAACCTCCACTTCCCGGGTTCAAGTGATTGTCCCACCTCA
GCCTCCTGAATAGCTGGGATTACAGGTGCATGCTACCATGCCTGGCTACT
TTTTGTATTTTTAGCAGAGACAGGGTTTCACCATGTTGGCCAGGGTGGTC
TCGAACTCCTGACCTCAAGTGATCCGTCTGGCTCAGCCTCCCAAAGTGCT
GGGATTACAGGTGTGAGCCACTGCACCTGGCCTATATAGGCTTTTTTCTT
AAACCTATTTAGTAATGTTTTCCCAAGTTTATTTTTTATTTTTAATTTTT
TCCCCAAGTTTATTTTTCTATTTTTTTTTCATGGAAAAATGGGGTAACTT
AGCAGTTTCAATATTGAAGACTGAAGTTTAAAAAAAATTTAAATTCAAGG
TACTTTTAAAATTCAGTTAGAAAAGTAGGCTTTAAAAATTATTAGAGACA
AGAGTACCAAAGCGGTGTGTGTATGTGTGTGTGTGTATGCATGCTTGTGG
ATTGGAAAAACTTTGGAGACTGATTACTTTTCATTATATATGTGTCACAG
TGAAACAGCTTTTATGTGTCATGTAAGATTACTGCTTGCCTCTCTAAGGA
AGGTCGTGACTGTTTAAATAGACGGGCAAGGTGGAACCTTTTGAAAGATG
AGCTTTTGAATATAAGTTGTCTGCTAGATCATGGTTTGTATTGAACTAAC
AAGGTTTGCAGATCTGCTGACTTATATAAAGCTTTTTGATTCCTACTAAG
CTTTAAGATTTAAAAAATGTTCAATGTTGAAATTTCTGTGGGGCTCTATT
TTTGCTTTGGCTTTCTGGTGAGAGAGTGAGGAAGCATTCTTTCCTTCACT
AAGTTTGTCTTTCTTGTCTTCTGGATAGATTGATTTTAAGAGACTAAGGG
AATTTACAAACTAAAGATTTTAGTCATCTGGTGGAAAAGGAGACTTTAAG
ATTGTTTAGGGCTGGGCGGGGTGACTCACATCTGTAATCCCAGCACTTTG
GGAGGCCAAGGCAGGCAGAACACTTGAAGGAGTTCAAGACCAGCGTGGCC
AACGTGGTGAAACCCTGTCTCTACTAAAAATACAAAAATTGTTTAGCTCT
GTTTTTCATAATAGAAATAGAAAAGGTAAAATTGCTTTTCTTCTGAAAAG
AACAAGTATTGTTCATCCAAGAAGGGTTTTTGTGACTGAATCAGCAGTGC
CTGCCCTAGTCATAGCTGTGCTTCAAAAACCTCAGCATGATTAGTGTTGG
AGCAAAACAAGGAAGCAAAGCAAATACTGTTTTTGAAATTCTATCTGTTG
CTTGAACTATTTTGTAATAATTAAACTTTGATGTTGAGAAATCACAACTT
TATTGTACACTTCATTGCAACTTGAAATTCATGGTCTTAAAGTGAGATTT
GAATTTCTATTGAGCGCCTTTAAAAAAGTAATACCAAACCATAAAGTTAA
AATCTATGTATATTGAGTCATATCTAAAACCACGTATAAACATAAATTGT
ATTTCCTGTTTTAATTCCAGGGGAAGTACTGTTTGGGAAAGCTATTATTA
GGTAAATGTTTTACAAATTACTGTTTCTCACTTTCAGTCATACCCTAATG
ATCCCAGCAAGATAATGTCCTGTCTTCTAAGATGTGCATCAAGCCTGGTA
CATACTGAAAACCCTATAAGGTCCTGGATAATTTTTGTTTGATTATTCAT
TGAAGAAACATTTATTTTCCAATTGTGTGAAGTTTTTGACTGTTAATAAA
AGAATCTGTCAACCATCAAAGAGGTCTGCATTATGCTTGCATGTCAAAAA
CTTTAAAAATCCTATAATCTTCTGTCATTTTCACTGAGTTTCCATGGGAA
AGGAATAGTAAATAATGGGTAGTTGAAATATTACTCTTAAGACCAAGACC
TGTATCTCCAGTCATATCTGTAATAACATCATCTGATAACCTAAAAGCAT
AGTATTAGGGATATACGACAAAACCAAAGTGTTTTTGCTGTTGTCACATA
CCACTCAATACTTTTACACCAGGTTGTCCAGTGGACACCAGCTGGATGTC
CTATAATTCAATTCTGACACTGTCTACCTGGAATTGGAGTCAGATCCCAC
AGGTGGAGGGCTCAGCCCACAAGACTGCCCCCACTCCAGATACCAATCGC
AAGTCCCTATACTTCTGACTGGCTATAAAGTGGGGTTCCCGTGACCCTTT
CCTTGGTTTTGATTTATTTGCTAGAGCAGCTCCCAGAACACAGAGAAACA
CTCTACTTATGTTTACCCATTTTTAATTTTATTACTTTATTAAAACTACA
TATTGGTTACAATACTAATTTATTGTAAAGGATATTTCATTTATTTATTT
TTTTATTTAGAGATGGAGTCTCACTGTGTCACCCAGGCTGGAGTGCGGTG
GCACAGTCTCAACTCACTGCAACCTCCACCTCCTGAGTTCAAGCGATTCT
CATGCCTCAGCCTCCCAAGTAGCTGGGATTACAAGTGTGCACCACCATGC
CCAGCTAATTTTTGTATTTTTAGTAGAGATGGGGTTTCACTCTGTTGGCC
AGGCTGGTCTCAAACTCCTGACCTCAAGTCATCCACCCACCTCAGCCTCC
CAAAGTGCTGAGATTACAGGTGTGAGCCATTGCCTCTGGCTTGTAAAGGA
TATTTCAAAGAATACAGATGAACAGCCAGATGGAAGAGATGCACAGGGCA
AGGCATGTGGGAAGGGGTATGGAGCTTTCATGCTCTCTCCACGTCATCAG
CTACCCAGAAGCTCATCTGGACCCCGACCCTTTGGGGTTTTACGGAGGCT
TCATTATGTCAGCATTACCATTGGCAATTGGCGATTAACTCAACCTTCAG
CCTCTCTCCCCTTTCTGGAGGTTGGGGGTTGAGGCTGGGAGTCCAAACCC
TCCAATCTTGCCTTGGTCTTTCTGGTAAGCAGCCCCCCGTCCTGAAGCTA
CCTAGAGGCTCCCAGCCACCAATCATTTCATTGGCAAACCAAAGATACTT
CAAGGGGTTTAGGAGCCAAGAGACCAAGCAAGGATTGAGACAATATATAT
ATTTCTTATAAATAACATGATCACAAATAGTATTTTATATATTTCTCATA
ATAGCTGGTTGTGGTTACTCACACCTATCATCCCAGCTACTAAGAAGCTA
AGGCAGGAGGATCACAGGCCCAGGAGTTTGCAGCTGTAGTGAGCTATGAT
TGTGCTACTGCACTCCAGCCTGGGTGACAGTGTGAGACCCTGTCTCTTAA
AGTTCTTGTAACATCTTAGAACATTAACCAACCACAGATTATTGCCTTAA
CTCTGAAAAATAAAGTAATCATACTTTTACAAGATGTATTTTACATAAAG
TTCTGGTGATATTTTACTCTGGTCATTGGAATATAGGAGCTTACCACCTG
GTACCACTCTTCCTAAAGTGGTATACTATTTGGTATAGTACTGGCATACT
ATTTGGAAAGGGATGTGAAGTTGGCATGACCTGTCTAGGGTGAAGTCATG
CTGTCTGCTATTCATTGTGGGCTTTACAGTAATGAGAGTGCTTCTGTTGT
CAAGATGCTTCTGGGACACGTTAAAAAAAATTTAAGATTTCTCAGTTTGT
GAAAAGGAAGGAGCACGAATGCTTTCAAGTTCCTATGACATGCTAGTCAT
CTTTCAGTGTGTCCCATTGTTGCACCCAGTAGTCTCGATGCCCAGCGTCA
CGGTGATTCCAAGTTTGCCTCTCTGCCCACTATAATTTGTGCTCCATGAA
GTCCCAGGACATGAGGTTCTTGTTCATTTGTGCGTCTGGGCCCATATCCA
AATATTAAACAATGTGACTTAATGTATTTATCATATTGTAAATATGGGTA
TCCCCAAGGCTCAGAAGTTAACACAGCATATCCACAGAGAGGTTTGACAG
CATGGCTGTGGTGACTGGGAAGGCCTTATGCTTGCTCCTCCTTCTGCTGC
TTGCCTTTGTGTGGGCATGGAAAGAACATTGAATTAGGTACTGGAAGACC
TGGGTCCTGGTCTTGGAGATTTTACCAGAGCACCTGTGGGATATTGGTCA
AGATGCCTGGTTTTAGTCCTGCTCCTTTGCTGTGAAAGAGGATCTTAAGG
TGCCAACTCTGGCATTTTATTTTTTACTTATTTATTTTTAAATTATACTT
TAAGTTCTAGGGTACATGTGTACAACATGCAGGTTTGTTACATGTATACA
TGTGCCATGTTGGTGTGCTGCACCTATTAACTCGTCATTTACATTAGGTA
TATCTCCTAATGCTATCCCTCCCCCTTCTCCCCACCCCTCGACAGGCCCC
GGTGTGTGATGTTCCCCTTCCTGTGTCCAAGTGTTCTCATTGTTCAATTC
CCATCTATGAGTGAGAACATGCGGTGTTTGTTTTTTTGTCCTTGGAATAG
TTTGCTGAGAAGGATGGTTTCCAGCTTCATCCATGTCCCTATAAAGGACA
TGAACTCTTCCTTTTTTATGGCTGCATAGTATTCCATGGTGTATATGTGC
CACATCTTCTTAAACCAATCTATCATTGATGGACATTTGGGTTGGTTCCA
AGTCTTTGCTATTGTGAACAGTGCCACAATAAATATACGTGTGCATGTGT
CTTTATAGCAGCATGATTTATAATCCTTTGGGTATATACCCAGTAATGGG
ATGGCTGGGTCAAATGGTATTTCTAGTTCTAGATCCCTGAGGAATCGCCA
CACTGTCTTCCACAATGGTTGAACTAGTTTACAGTCCCACCACCAGTGTA
AAAGTGTTCCTATTTCTCCACATCCTCTCCAGCACCTGTTGTTTCCTGAC
TTTTTAATGATTGACATTCTAACTGGTGTGAGATGGTATCTCATTGTGGT
TTTGATTTGCATTTCTCTGATGGCCCATGATGATGAGCATTTTTTTCATG
TGTCTGTTGGATGCATGGATGTCTTCTTTTGAGAAGTGTCTCTTCATATC
CTTCACCTACTTTTTGATGGGGTTTGTTTTTTTCTTGTAAGTTTGTTTGA
GTTCCTTGTAGATTCTGGATATTAGCCCTTTGTCAGATGAGTAGATTGCA
AAAATTTTCTCCCATTTTGTAGGTTGCCTGTTCACTCTGATGGTAGTTTC
TTTTGCTGTGCAGAAGCTCTTGAGTTTAATGAGATCCCATTTGTCAATTT
TGGCTTCTGTTGCCATTGCTTTTGGTGTTTTAGACATGAAGTCCTTGCCC
ATGCCTGTGTCCTGAATGGTACTGCATAGGTTTTCTTCTAGGGTTTTTAT
GGTTTTAGGTCTAACATTTAAGTCTTTAATCCATCTTGAATTAATTTTTG
TATAAGGTGTAAGGAAGGGATCCAGTTTCAGCTTTCTACATGTGGCTAGC
CAGTTTTCCCAGCACCATTTATTAAATAGGGAATCCTTTCCCCATTTCTT
GTTTTTGTCAGGTTTCTCAAAGATCAGATAGTTGTAGATGTGTGGTATTA
TTTCTGAGGGCTCTGTTCTGTTCCATTGGTCTATATCTCTGTTTTGGTAC
TAGTACCATGCTGTTTTGGTTACTGCAGCCTTGTAGTATAGTTTGAAATC
AGGTAGCGTGATGCCTCCAGCTTTGTTCTTTTGGCTTAGGATCGTTTTGG
CAGTGCAGGCTCTTTTTTGGTTCCACATGAACTTTAAAGTAGTTTTTTCC
AATTCTATGAAGAAAGTCATGGGTAGCTTGATGGGGATGGCATTGAATCT
ATAAATTACCTTGGGCAGTATAGCCATTTTCACGATATTGATTCTTCCTA
TCCATGAGCATGGAATGTTCTTCCATTTGTTTGTGTCCTCTTTTATTTCC
TTGAGCAGTGGTTTGTAGTTCTCCTTGAAGAGGTCCTTCACATCCCTTGT
AAGTTGGATTCCTAGGTATTTTATTCTCTTTGAGGCAGTTGTGAATGGGA
GTTCACTCATGATTTGGCTCTCTGTTTGTCTGTTGTTGGTGTATAAGAAT
GCTTGTGATTTTTGCACATTGATTTTGTATCCTGAGATTTTGCTGAAGTT
GCTTATCAAATTAAGGAGATTTTGGGCTGAGATGATGGGGTTTTCTAAAT
ATAAAATCATGTCATCTGCAAACAGGGACAATTTGACTTCCTCTTTTCCT
TATTGAATACCCTTTATTTCTTTCTCCTGCCTGATGGCCCTGGCCAGAAC
TTCCAACACTATGTTGAATAGGAGTGGTGAGAGAGGGCATCCCTGTCTTG
TGCCAGTTTTCAAAGGGAATGCTTCCAGTTTTTGCCCATTCAGTCTGATA
TTGGCTGTGGGTTTGTCATAGATAGCTCTTATTATTTTGAGATATGTCCC
ATCAATACCTAATTCATTGAGAGTTTTTAGCATGAAGGGCTGTTGAATTT
TGTCCAAGGCCTTTTCTGCATCTGTTGAGATAATCATGTGGTTTTTGTCT
TTGGTTCTGTTTATATGCTGGATTACTTTTATTGATTTGTGTATGTTGAA
CCAGCCTTGCATCCCAGGGATGAAGCCCACTTGATCATGGTGGATAAGCT
TTTTGATATGCTGCTGGATTCAGTTTGCCAGTATTTTATTGAGGATTTTT
GCATTGATTTTCATCAGGGATATTGGTCTAAAGTTCTCTTTTTTTGTTAT
GTCTCTGCCAGGCTTTGGTATCAGGATGATGCTGGCTTCATAAAATGAGT
TAGGGAGGATTCCCTCTTTTTCTATTGATTGGAATAGTTTCAGAAGGAAT
GGTACCAGCTCTTCCTTGTACCTCTGGTAGAATTCGGCTGTGAATCCATC
TTGTCCTGGACTTTTTTTGGTTGGTAGGCTATTAATTATTGCCTCAATTT
CAGAGCCTGTTATTGGTCTATTCAGGGATTAAACTTCTTCCTGGTTTAGT
TTTGAGAGGGTGTATGTGTCCAGGAATTTATCTATTTCTTCTAGATTTTC
TAGTTTATTTGCATAGAGGTGTTTATAGTATTCTCTGTGTACTCTGTATT
TCTGTGGGATTGGTGGTGATATCCACTTTATCATTTTTTATTGCATTTAT
TTGATTCTTCTCTCTTTTCTTGTTTATTAGTCTTGCTAGCGGTCTATCAA
TTGTGTTTATCTTTTCAAAAAACCAGCTCCTGGATTCATTCATTTTTTGA
AGGTTTTTTTGTGTCTCTGTCTCCTTCAGTTCTGCTCTGATCTTAGTTAT
TTCTTGTCTTCTGCTAGCTGTTGAATGTGTTTGCTCTTGCTTCTCTAATA
TTTTTAATTGTGATGTTAGAGTGTCAATTTTAGATCTTTCCTGCTTTCTC
TTGTGGGCACTTAGTGCTGTAAATTTCCCTCTACACACTGCTTTAAATGT
GTCCCAGAGATTCTGGTATGTTGTGTCTTTGTTCTCGTTGGTTTCAAAGA
ACATCTTTATTTCTGCCTTCATTTTGTTATGTACCCAGTAGTCATTCAGG
AGCAGGTTGTTCAGTTTCCATGTAGCTGAGTGGTTTTGAGTGAGTTTCTT
AATCCTGAGTTCTAGTTTGATTGCACTGTGGTCTGAGACAGTATGTTATA
ATTTCTGTTCTTTTACATTTGCTGAGGAGTGCTTTACTTCCAACTATGTG
GTCAATTTTGGAATAGGTGCGATGTGGTGCTGAGAAGAATGTATATTCTG
TTGATTTGGGGTGGAGAGTTCTGTAGATGTCTATTAGGTCTGCTTGGTGC
AGAGCTGAATTCAATTCCTGGATATCCTTGTTAACTTTCTGTCTCGTTGA
TCTGTCTAATATTGACAGTGGGGTGTTAGAGTCTCCCATTGTTATTGTGT
GGGAGTCTAAATCTCTTTGTAGGTCTCTAAGGACTTGGTTTATGAATCTG
GGTGCTCCTGTATTGGGTGCATATATATTTAAGATAGTTAGCTCTTCTTG
TTGAATTGATCCCTTTACTATCATGTAATGGCCTTCTTTGTCTCTTTTGA
TCTTTGTTGGTTTAAAGTCTGTTTTATCAGAGACTAGGATTGCAACCCCG
GCCTTTTTTTTTGTTTTCCGTTTGCTTGGTAGATCTTCCTCCATCCTTTT
ATTTTGAGCCTATGTGTGTGTGTCTCTGTACGTGAGATGGGTTTCCTGAA
TACAGCACCCTGATGGGTCTTGACTCTTTATCCAGTGTGCCAGTCTGTGT
CTTTTAATTGGAGCATTTAGCCCATTTACATTTAAGGTTAATATTGTTAT
GTGTGAATTTGATCCTGTCACTAGGATGTTCACTGGTTATTTTGCTCGTT
AGTTGATGCAGTTTCTTCCTAGCCTCAATGGTCTTTACAATTTGGCATGT
TTTTGCAGTGGCTGATACTGGTTGTTCCATTCCATGTTTAGTGCTTCCTT
CAGGAGCTCTTGTAAGGCAGGCCTGGTGGTGACAAAATCTCTCAGCATTT
GCTTGTTTGTAAAGGATTTTATTTCTCCTTCACTTATGAAGCTTAGTTTG
GCTGGATATGAAATTCTGGGTTGAAAATTCTTTTCTTCAAGAACGTTGAA
TATTGACCCCCACTCTCTTCTGGCTTGTAGAGTTTCTGCTGAGAGATCAG
CTGTTAGTCTGATGGGCTTCCCTTTGTGGGTAACCCGACCTTTCTCTCTG
GCTGCCCTTAACATTTTTTCCTTCATTTCAACTTTGGTGAATCTGACAAT
TATGTGTCTTGGAGTTGCTCTTCTCGAGGAGTATCTTTGTGGCGTTCTCT
GTATTTCCTGAATTTGAATGTTGTCCTCCCTTGCTAGATTGGGGAAGTTC
TCCTGGATAATATCCTGCAGAGTGTTTTCCAGCTTGGTTCCATTCTCCCC
GTCACTTTCAGGTACACCAATCAGACGTAGATTTGGTCTTTTCACATAGT
CCCATATTTCTTGGAAGCTTTGTTCGTTTCTTTTTACTCTTTTTTCTCTT
AACTTCTCTTCTCACTTCATTTCATTCATCTGATCTTCAATCACTGATAA
CCTTTCTTCCAGTTGATCGAATTGGCTACTGAAGCTTGTGCATTCGTCAC
GTAGTTCTCATGCCATGGTTTTCAGCTCCATCAGGTCATTTAAGGACTTC
TCTATGCTGGTTATTCTAGTTAGCCATTTGTCTAATCTTTTTTCAAGGTT
TTTAGGTTCTTTGTGATGGGTTCGAACTTCCTCTTTTAGCTTGGAGAAGT
TTGATCATCTGAAGCCTTCTTCTCTCAACTCGTCAAAGTCATTCTCCGTC
CAGCTTTGTTCTGTTGCTGGCAAGGAGCTATGTTCCTTTGGAGGGGGAGA
GGCGCTCTGATTTTTAGAATTTTCAGCTTTTCTGCTCTGTTTTTTCCCCA
TCTTTGTGGTTTTATCTACCTTTGGTCTTTGGTGATGGTGATGTGCAGAT
GGGGTTTTGGTGTGGATGTCCTTTCTGTTTGTTAGTTTTCCTTCTAACAG
TCAGGACCGCCAGCTGCAGGTCTGTTGGAGTTTGCTGGACGTCCACGCCA
GACCCTGTTTGCCTGGGTATCACAGTGGAGGCTGTAGAACAGCGAATATT
GCTGAACAGCAAATGTTGCTGCCTGATCATTCCTCTGGAAGCTTCATCTC
AGAGGGGTACCCGGCCATGTGGGGTGTCAGTCTGCCCCTACTTGGGGGTG
CCTCCCAGTTAGGCTACTCGGGGGTCAGGGACTCACTTGAGGAGGCAGTC
TCTCCGTTCTCAGATCTCAAACTCTGTGCTGGGAGAACCACTGGTCTCTT
CAAAGCTGTCAGACAGGGACATTGAAGTCTGCAGAGATTTCTGCTGCCTT
TTGTTCGGCTATGCCATGCCCCCAGAGGTGGAGTCTACAGAGGCAGGCAG
GCAGGCCTCCTTGAGCTGCAGTGGGCTCCACCTAGTTCGAGCTTCCCGGC
TGCTTTATTTACCTACTGAAGCCTCAGCAATGGTGGGCTCCCCTCCCCTA
GCCTCACTGCCACCTTGCAGTTCGATCTCAGACTGCTGTGCTAGCAATGA
GCAAGGCTCCATGGGCGTGGGACCCTCCGAGCTAGGCACAGGATATAATC
TCCTGGTGTGCCGTTTGCTAAGACCATTGGAAAAACGCAGTATTAGGGTG
GGAGTGACCCAATTTTCCAGGTGCCGTCTGTCACAGCTTCCCTTGGCTAG
GAAAGGGAATTCCCTGACCCCTTGCCTTCCCGGGTGAGGCCATGCCTTGC
CCTGCTTCAGCTCATGCTCAGTGGGCTGCACCCACCGTCCTACACCCACT
GTCCAACAAGCCCCAGTGAGATGAACCTGGTACCTCAGTTGGAAGTGCAG
AAATCACCTGTCTTCTGCGTCGCTCACGCTGGGAGCTGTAGACTGGAGCT
GTTCCTATTCGGCCATCTTGGAACCCAACCCAAGTCTGGCATTTTTTGGT
TTTCTCTGTGTATTGACTATGATTATAGAGGATAAATGGATATTGATATT
ATTAAATCGTCATTTACAGAAGGCACACAACTTATATCCTTTAAACAAAG
TTCTCCAACCAGTAGGGTATACTTGCTGGTAGTGCTTACAGCTGCCACCA
TTCTTCACTGGGGCTCAGAGTGGATGCCTGGAGGAAAAAAACAAATGAAT
CTTTTAGCTCCTAGTTACTGTTAGCATTTTTACCAGAAAACAATTTAAAA
AATGACACAAGTAAGTCTTCACCTATCAAGGCTGAGTGTGGTGGCCTATA
CTTGTAATCTCTGTGCTTTGGGAGGCCAACGCAGGAGGATCTCTTGAGTC
CAGGAGTTTGAGACCAGCCTGGGCAACAAAGCAAGACCCCATCTCTACAG
AAAGTTAAAATTTAGCTGGGCATGCTGGCACATCCCTATAGTCCCAGCTA
CTTGAGAGGCTGAGTTGGGAGGATCCCTTGAGGTCATGAGTTGGAGTCTA
CAGTGAGCTGTGATTACAGTGTGAGTCTACACTGCACTCCAGTCTGGGTG
AAAGAGATCCTGTCTCTCAAAAAACAAGTGATTCTCCTGGGAAGAGATTT
GTTTAAAAAGTCTGCACCTCTATTGATGATAACTTTGAATGTAAATGGAT
TAAATTCTCCCACTTAAAAGATATAGATTGACTGAATGGATTAAAAACCA
CCATCATGACTCGACTGTATGCTGCCTACAAGAAACCTCACCTGTAAAGA
CAGGCCAAAAGTGAAGGTATGGAGAAAGATATTGCATACAAATGGAAGCC
AAAAAAGAGCAGAAATAACTATACTTACATGAGATAAAATAGACTTTAAG
TCAAAAACTGTAAAAAGAAAAGTCATTATGTAATGAAACAAGGATCAATT
CATGCAAGATATAATCATATTTTTTTATTAAGAAAATTTTTTTGTAGAGA
TGGGGTTATGCTATGTTGCCTGGCTAGTCTCAAACTCTGGGTCTCAAAGC
AGTCCTCCTGCTTTGGCCTCCTAAGGTGCTGGGATTATAGGCATGAGCCA
CTGTGCCAGGCCTGAGGATATTATCATTATAAATATATATGTACCCCACA
CTGGATGACCCAGATATAAAGCAAATATTATTAGATTTAAAGGGAGAAAT
AGACTCTAATACAATAGTAGTTGAGGACTTCACTCTACTCTCAGCATTGG
ACAGATTTTCTAGACATAAAACCAATAAATATTGGATTTAAATTGCACTT
TACACCAAATGGACCTGACATTTACAGAACATTCTAATAGGTGCAGAACA
CTCTAATAGCTGCAGAACACACGTTCTTTTCATCAGCACATGGAACATTC
ACCAGACTAGTTCTGGAACCCACTTCCTGCCTTAGTGTTCTTTCCACCAC
AGTGCATGATAATTCCGACAGAACGCCTTTTATTTGTACCTTATTAGTTG
TAGGGGGAAAGCTTTTTCATGTACAAAAATGCTGTAATTTTCTGATTTAA
TAAAGTAATTCCACTGA
SOD2 Gerbil Sequence
(SEQ ID NO: 10)
TCGCCGTCCTCCCCTCCGCCGATGGGAGCGGCCGGCAGGGTCGCCGCCCT
GAAGTGTCCTTGTAGAGGCGCCTCGGACGCCGCGGAGCAGACGCACGCCT
GCGAGCGGACCGTTGACTTGTCCGGACACCGGAGGCCGTGGGCATCTCAG
CAATGCTGTGTCGGGCGGCGAGCAGCGCGGGCAGGAGGCTGGGCCCGGCG
GCCAGTGTCGCGGGCTCCCGGCACAAGCACAGCCTTCCCGACCTGCCTTA
CGACTATGGCGCGCTGCAGCCGCACATCGACGCGCAGATCATGCAGCTGC
ACCACAGCAAGCACCACGCGGCCTACGTGAACAATCTGAACGTCACCGAG
GAGAAGTACCACGAGGCACTGGCCAAGGGAGATGTTACGGCTCAGATTGC
TCTTCAGCCAGCACTGAGGTTCAATGGTGGGGGCCATATTAATCACTCCA
TATTCTGGACGAACTTGAGCCCTAATGGTGGTGGAGAACCCAAAGGAGAG
TTGCTGGAGGCTATCAAACGTGACTTTGGGTCTTTTGAGAAGTTTAAGGA
GAAGCTGACAGCTGTGACTGTTGGAATCCAAGGTTCAGGCTGGGGCTGGC
TTGGCTTCAACAAGGAGCAGGGTCGCTTGCAGATTGCTGCCTGCGCTAAC
CAGGATCCATTGCAAGGAACAACAGGCCTTATTCCATTGCTGGGGATTGA
TGTGTGGGAACATGCCTATTACCTTCAGTATAAAAACGTCAGACCCGATT
ACCTGAAAGCTATTTGGAATATAATCAACTGGGAGAATGTCACTGAAAGA
TACACAGCTTGCAAGAAGTAAAGCCTCATCACCATATTGAGCATATCAGG
CCCTTGATGGCTACTTTTGTAGTAGTATAAAGTGCTGCAGCTACTGTGAC
TGCGCTGTTGTTGACATTTATTGATGTACGTCCACATAGCTGATGACTAG
GATGTTAAGATAATTTCTTATTCTTAATTAAACTTACAATTAGGCAACTG
TTTGAGAACAGTACATATTCTGTGTGAATTATTCTTGATTGAACATTTTC
ATTTGAACCTTGAATTGCTAGGATGCTCTGTCATTGTAAGACTACCAAAA
ATATTCTATCTCAATGTTTTGGGGGCCTACTGGAGAGTCTAATCCTGTTC
TACTGCAGTTAGAAAAAAAATGGAGTTGTTCCCCCACCTCGAGTTGTTAA
ACAATAAAATAGTTCATTTTCTATTAAAAATTGCTATTTTTGGTAAGTAA
GCCTTTGCTTAGTAGATATCACCTAGTGGTCTTTATTTATGGTCACAGTT
CTGCATGAGAAACACAATTTTTTTATTTGAAATCTGTAACTTGAGGCTAA
GGATGTAGTTCAGTGGTAGAGCTTTAGTGTGCAAAGCCCTGGGTTCTACT
TCAGTACTGAAAACAAATCAACAGAACCCATAGCTTGGTTACTGAGTGAG
AACTGTTTTATTATAAGAGGAGTCTAATTGTGCCTCTGGGGTTTTTCTAT
AGGCAGAGATGGAAAAACATTTAATATTGTCTTTAAAAACCAATTGTATG
AAGTGTTCAACGTGGACTATTAGGGCCTGCCTGATGATGTCAACCATGGG
GACTGGGCCACAGGGCATTCTGGGAAGCCACCTAGCCCCCTTAATTGTGC
TGGAGAACTGGACCCTGGGCCAGATGGCACATTTCTCAGTAGAGGCAAAT
GGATTTCTGAGTGAAGTTAGAAGGGTGTCTTCAGAACATTAGTTTTCAGA
GACATTTATTTTTATTTTATCTATAGTTTCAAAATTTTATAGCTACCAGA
GGGGCTTTATATTATGTTCTTCAGTAAAGGGAGGGACTTTCCATGACAAG
ATGACACAGTGTTTCTGATTGTGTTCCTGTGCAGAGTTGTATGTGGAGTT
GTGCCCTGTGGAAATGCCACAGGGGCCCTTGTGAATTTGCCTTTCATATT
TTGTTTTAGCATCTTCCTAACTGTCTTTAATGACTATGCAATGTTTTAAT
CAATTGGACATGCCATTTATAGTTCCATTACTGGGTAGTTCATTTGTGCT
TAATTGTTGTTTATTACCAAAAAGGCTGTGTCAGCAACCCCATGCATTTG
AAGTACTTCTTACTGATTGGGGTCTTTTTTAAGGTTCATCTTATTTGCAG
TTATGTGTGTCTGTGTATGTCTGTTCATATGAATGCAGGTGCCCATGGAA
GACAGCAGATAGTGTTGGATCTCTCAGAGCGGAAGTAGTTACAGTGGCCG
AAGCCACCTGATGTGGGTGTGGGACTTGGACTGGGGTCACCTGCAAGAGC
AATAAGCACTTAACCACTGAGCCACCTCTCCAGCCCCTGCATTTTTTCTT
TCTTTTTCTTTTTCTTTTTTTTTGGTTAAATACTGAGTCTTTTTTTTTCT
TCTTAAATCTATCTTGTAATTTATTCCAAGTTTTTTTTAATCCTCCCCTC
AAGTATATTTTCCTTTTTTTTTTAATGGAAAAATGGAGTGACTTAGAAGT
TTCAGTATTGAAGACTGAAGTTTAAAAAACAAAACCAAACTTAAATTCAA
AGCACTTTATAACAGAAGCTCATCAGGTGTTTTCAGCGTCAGTGCTGATC
GGTCCTGGTGGCTCCTTTTGTATGTTTATACTTGTGTGTGGAGGGACTCA
GAAGACTGAGTAGCAGCCAGTTGTATGGGACAGTGACAGGACTGAGCAAA
TGAGAGTCAACATGGTTCCACTTTGTTTACAAGGCAGTGAACTCTTTTAA
ACAGACTTTTTAGGAAAGTGGGACTTTGATGGATAGACTTTGGAAAACAA
GTCTTAAGATCAGTTTTCTTGAACTAACAAAGATTTGCAGAGACTACTGA
CTTTATAAAACTTTGACTCATACTAAGCTTTCAGATTAAAAAAAATTCAA
TGGTGAAATTTCTATGAGGCTCTATTTTTGCTGTGGCTTTCTGTTGTCAA
GAATGAGGAATGGCACCGTGTTCATTCGGTTAAAGTCTCCCGATGCATAG
ATTTTTAAGAGACCAGGAGAATTTACAAACTAAAATAATCCAGTGTAAAA
TAAGGCTTAGATTGGGAAGAAATATGTGTTAAGTGTGTGTGAGTATGTGT
GAGTGTGTGCAACAAGATCAGCAGTGTGTTGTCCCAGTCATGGTTGAACT
GACAGGCATCAGCATATACTGGGGAGAAACAAGGAAGCTATTTCCTTTGA
GGTAGTATCTGCTGCTTGAACTATTGTGCATTAATTAAACTTTGGAGTTA
AGAAATTGCCTTATTGCATTCTGAAATTCTTGGCCTACAGTGAGATCTGA
ATTTCTGTTAAGCACCTTTATAAAAAGTATTGAAGCCATATAGTCAGGAC
TGATATGTATTGTGTGTGGTGTCATAGTGAAAACTAGGTATAAATACTGG
CTTTTTTTTTCCTGTTGTAATTCAGTTAAACTGCTTTCAGGAACACTGTT
AGGTACGTTTTTAAATAAGTCACAGCAAAGCATATCCTGCTGATCCCAGG
AAGCTAGCATTTGTCTTCTGAACATCATGTCTAGCACCTGCTGAAAAGTC
TGTAAGGTAGTTTTCGCTTAAATATTGATCAAAGAAGTGTTTATTTTCCT
ATTGTAAGTGCAAAGTGGTGTTGATTGTTAATAAAAATAGTCTGTCAACT
ATCAA

In certain embodiments, the invention also provides an AAV vector comprising a nucleic acid encoding a SOD3 protein (alternatively known as MEC-SOD and MGC20077). Examples of sequences encoding SOD3 protein include, but are not limited to NCBI gene. 6649, HGNC, 11181, OMIM, 185490, RefSeq, NM_003102, UniProt, P08294. In certain embodiments, the nucleic acid encoding the SOD2 protein comprises the Homo sapiens chromosome 4, GRCh38.p14 Primary Assembly NCBI Reference Sequence: NC_000004.12>NC_000004.12: 24795573-24800842. In certain embodiments, the SOD3 protein is encoded by a nucleic acid comprising a polynucleotide sequence having at least 80%, 85%, 90%, 95%, 96%, 97%, 98%, or 99% identity to SEQ ID NO: 3. In certain embodiments, the SOD3 protein is encoded by a nucleic acid comprising the nucleotide sequence set forth in SEQ ID NO: 3.

SOD3 DNA (NM_003102)
(SEQ ID NO: 3)
AGGGACAGCCTGCGTTCCTGGGCTGGCTGGGTGCAGCTCTCTTTTCAGGA
GAGAAAGCTCTCTTGGAGGAGCTGGAAAGGTGCCCGACTCCAGCCATGCT
GGCGCTACTGTGTTCCTGCCTGCTCCTGGCAGCCGGTGCCTCGGACGCCT
GGACGGGCGAGGACTCGGCGGAGCCCAACTCTGACTCGGCGGAGTGGATC
CGAGACATGTACGCCAAGGTCACGGAGATCTGGCAGGAGGTCATGCAGCG
GCGGGACGACGACGGCGCGCTCCACGCCGCCTGCCAGGTGCAGCCGTCGG
CCACGCTGGACGCCGCGCAGCCCCGGGTGACCGGCGTCGTCCTCTTCCGG
CAGCTTGCGCCCCGCGCCAAGCTCGACGCCTTCTTCGCCCTGGAGGGCTT
CCCGACCGAGCCGAACAGCTCCAGCCGCGCCATCCACGTGCACCAGTTCG
GGGACCTGAGCCAGGGCTGCGAGTCCACCGGGCCCCACTACAACCCGCTG
GCCGTGCCGCACCCGCAGCACCCGGGCGACTTCGGCAACTTCGCGGTCCG
CGACGGCAGCCTCTGGAGGTACCGCGCCGGCCTGGCCGCCTCGCTCGCGG
GCCCGCACTCCATCGTGGGCCGGGCCGTGGTCGTCCACGCTGGCGAGGAC
GACCTGGGCCGCGGCGGCAACCAGGCCAGCGTGGAGAACGGGAACGCGGG
CCGGCGGCTGGCCTGCTGCGTGGTGGGCGTGTGCGGGCCCGGGCTCTGGG
AGCGCCAGGCGCGGGAGCACTCAGAGCGCAAGAAGCGGCGGCGCGAGAGC
GAGTGCAAGGCCGCCTGAGCGCGGCCCCCACCCGGCGGCGGCCAGGGACC
CCCGAGGCCCCCCTCTGCCTTTGAGCTTCTCCTCTGCTCCAACAGACACC
CTCCACTCTGAGGTCTCACCTTCGCCTTTGCTGAAGTCTCCCCGCAGCCC
TCTCCACCCAGAGGTCTCCCTATACCGAGACCCACCATCCTTCCATCCTG
AGGACCGCCCCAACCCTCGGAGCCCCCCACTCAGTAGGTCTGAAGGCCTC
CATTTGTACCGAAACACCCCGCTCACGCTGACAGCCTCCTAGGCTCCCTG
AGGTACCTTTCCACCCAGACCCTCCTTCCCCACCCCATAAGCCCTGAGAC
TCCCGCCTTTGACCTGACGATCTTCCCCCTTCCCGCCTTCAGGTTCCTCC
TAGGCGCTCAGAGGCCGCTCTGGGGGGTTGCCTCGAGTCCCCCCACCCCT
CCCCACCCACCACCGCTCCCGCGGGAAGCCAGCCCGTGCAACGGAAGCCA
GGCCAACTGCCCCGCGTCTTCAGCTGTTTCGCATCCACCGCCACCCCACT
GAGAGCTGCTCCTTTGGGGGAATGTTTGGCAACCTTTGTGTTACAGATTA
AAAATTCAGCAATTCA
SOD3 VARIANT 1 GERBIL
(SEQ ID NO: 11)
GGGTTATGTACCTCGGACAACCTCAGACAGAAATTACAAAACTCACACAA
AAATCCTACCCCATGTCTTTGCTGAACATATACAGGCCTTTCTTCTTGTG
ATGGCTATTTACATAGCATTTACACTGCTAACTTCAATAAGCCGTCCAGG
CATGTTTTAAAGGACACAGGGCCATCTGAGCTGTTTCTGGGCACAGTTCT
TGCTATCTTACATAAGGAACTTGGGCACCCATGAATTTTGGAATCCTTGA
AGCATCCTGGAATCAGTCACCCACATATGAAGTGAGTCAGCTGTGTTGTG
ACTAAGGTTGAATTCTGGACCTTTCCATTGTCTTTGATTGCGCACCAGGA
TATTTAAACTTTCAAAAAGGCAAACGACAGCTAAGCTCTTCTGAATCCAG
GCTCTCGGGTGGTGGCCTCTTGTGGATCTTGGTTTTAGAGTCTTGGATCC
AGGCTGGGAAGATGGACTGGTGCAGGACCTCAGCCATGCTGGCCTTCCTG
TTTTGTGGCTTGCTTCTGGCGGCCCATGGCTCTGTCACCTGGACCATGCT
GGATCCCGGGGAGTCCAGCTTCGACCTAGCAGAGATCAAGACTGACCTGT
TTGAGAAGATAAGCGACACTCACGCCAAAGTGCTGGAGATCTGGCTGGGG
CTGGGACGGCGGGAGGTGGATGCCCCTGAGTTGTACGCGGCCTGCAGGGT
GGAGCCGTCAGCCACGCTGGCAGCCGATCAGCCTCGGGTCACCGGTCTGG
TCCTCTTCCGGCAGCTAGCGCCGGGCGCCAAGCTGGAGGCCTTCTTCAAT
CTGGAGGGCTTCGATGACGAGCTGAACGTGTCCAGCCACGCCATCCACGT
GCACGAGTTCGGGGACCTGAGCCAGGGCTGTGAGTCCACCGGGCCGCACT
ACAACCCGCTGGCCGTGGCGCACCCGCAGCACCCGGGCGACTTCGGCAAC
TTCGTGGTGCGCGATGGGCGCCTCTGGAAGTACCGCTCTGGCTTGGTCGC
TTCGCTGGCGGGCCCGTACTCCATCTTGGGCCGTGCCGTAGTGGTCCACG
CTGGCCAGGATGACCTGGGCCGCGGCGGCAACCAGGCCAGCGTGGAGAAC
GGCAACGCGGGGCGCCGGCTCGCCTGCTGCATAGTGGGTGCCAGCAGCTC
TGCGGCCTGGGAGCGCCAGGCCAAGGAGCACACCGAGCGTAAAAAGCGGC
GGCGGGAGAGCGAGTGCAAGACCACCTAAGCGGCACTCCCAGCCGCAGGG
CCCAGCTGCTTCACGCATAGATGCCTCCACAGGTCCCGGACACCACCCTC
ACTCCAGAGGCCTCCACAGTCCTAGACAGATGCCTTCCAGACGCCACAGT
CGCCTCTGCGCGCCCCACATGCTTCCAGGCACTCCAGGCACCCCTGTGTG
CTCCCAGACGGCTTCACGGAACTCCCCAGTCACCTCTCGGTGCCCCATTT
GTTCCCACGTGCCCTCGACACCCTTCCTGTGTGTCCCAGGACAGCTTGAG
TAACCCAGGAAACTTTTCATGCCCTAGGCACTTTCACAGACCCAGAACTC
CTTCATACCCCAGACCCACCTCAAGAGTTCCCCTGTGTCCCAAGCCTTTG
AAAGAGTCTTTGAGTCTGTTTGCTGCCGGAGAACCCCCTCTCCCCAGGCT
GCACGTGCTCAGACGCTCCCCTTCCACGCTGAGGACCTCGATGGCAGCAC
CTGAGTACATCCTCCTCAGTTGTGCGGAAATCCATTTCCTCCTGTCCATC
TTTTCCTTCCTATCCCCCAGCAACCGACACGAGGGACTTTTTTTCCCTTT
TGTTCCTCCTAGATGCCCAGAGACCATCCCAACACACACACACACACACA
CACACACACACACACACACACCTAGGATTCCATGTCCCACACCACCTCCT
GCGGTGCCCCCGGCTCGCTTTTCAGCTGTTTCCCACATGGTGCCTGCACC
CTGTGCAGAGAGGCTCCCATGAGAGTACTTGGCAACCTTTGTGCCGTACA
TTAAAAACACAGCAATTCAGTCCTGCA
SOD3 VARIANT 2 GERBIL
(SEQ ID NO: 12)
GCTGCTCACAAACAGCCAGCCGGCCAGCCAGCCAGCCCTGGGGAGGCAGC
ACATAGGTTCTCCCTCAGGCTTCTAGCTGGGTGCTGCCCCCCACTTCGCC
AGAGGGAAAGCGCTCTTGGGAGAGCCTGACAGGTGCAGGACCTCAGCCAT
GCTGGCCTTCCTGTTTTGTGGCTTGCTTCTGGCGGCCCATGGCTCTGTCA
CCTGGACCATGCTGGATCCCGGGGAGTCCAGCTTCGACCTAGCAGAGATC
AAGACTGACCTGTTTGAGAAGATAAGCGACACTCACGCCAAAGTGCTGGA
GATCTGGCTGGGGCTGGGACGGCGGGAGGTGGATGCCCCTGAGTTGTACG
CGGCCTGCAGGGTGGAGCCGTCAGCCACGCTGGCAGCCGATCAGCCTCGG
GTCACCGGTCTGGTCCTCTTCCGGCAGCTAGCGCCGGGCGCCAAGCTGGA
GGCCTTCTTCAATCTGGAGGGCTTCGATGACGAGCTGAACGTGTCCAGCC
ACGCCATCCACGTGCACGAGTTCGGGGACCTGAGCCAGGGCTGTGAGTCC
ACCGGGCCGCACTACAACCCGCTGGCCGTGGCGCACCCGCAGCACCCGGG
CGACTTCGGCAACTTCGTGGTGCGCGATGGGCGCCTCTGGAAGTACCGCT
CTGGCTTGGTCGCTTCGCTGGCGGGCCCGTACTCCATCTTGGGCCGTGCC
GTAGTGGTCCACGCTGGCCAGGATGACCTGGGCCGCGGCGGCAACCAGGC
CAGCGTGGAGAACGGCAACGCGGGGCGCCGGCTCGCCTGCTGCATAGTGG
GTGCCAGCAGCTCTGCGGCCTGGGAGCGCCAGGCCAAGGAGCACACCGAG
CGTAAAAAGCGGCGGCGGGAGAGCGAGTGCAAGACCACCTAAGCGGCACT
CCCAGCCGCAGGGCCCAGCTGCTTCACGCATAGATGCCTCCACAGGTCCC
GGACACCACCCTCACTCCAGAGGCCTCCACAGTCCTAGACAGATGCCTTC
CAGACGCCACAGTCGCCTCTGCGCGCCCCACATGCTTCCAGGCACTCCAG
GCACCCCTGTGTGCTCCCAGACGGCTTCACGGAACTCCCCAGTCACCTCT
CGGTGCCCCATTTGTTCCCACGTGCCCTCGACACCCTTCCTGTGTGTCCC
AGGACAGCTTGAGTAACCCAGGAAACTTTTCATGCCCTAGGCACTTTCAC
AGACCCAGAACTCCTTCATACCCCAGACCCACCTCAAGAGTTCCCCTGTG
TCCCAAGCCTTTGAAAGAGTCTTTGAGTCTGTTTGCTGCCGGAGAACCCC
CTCTCCCCAGGCTGCACGTGCTCAGACGCTCCCCTTCCACGCTGAGGACC
TCGATGGCAGCACCTGAGTACATCCTCCTCAGTTGTGCGGAAATCCATTT
CCTCCTGTCCATCTTTTCCTTCCTATCCCCCAGCAACCGACACGAGGGAC
TTTTTTTCCCTTTTGTTCCTCCTAGATGCCCAGAGACCATCCCAACACAC
ACACACACACACACACACACACACACACACACACCTAGGATTCCATGTCC
CACACCACCTCCTGCGGTGCCCCCGGCTCGCTTTTCAGCTGTTTCCCACA
TGGTGCCTGCACCCTGTGCAGAGAGGCTCCCATGAGAGTACTTGGCAACC
TTTGTGCCGTACATTAAAAACACAGCAATTCAGTCCTGCA

### Gene Transfer Systems and Adeno-Associated Virus (AAV)

Gene transfer systems, such as those described in the present disclosure, depend upon a vector or vector system to shuttle the genetic constructs into target cells. Methods of introducing a nucleic acid into a cell, including cells of the inner ear, include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II [Amaxa Biosystems, Cologne, Germany]), (ECM 830 (BTX) [Harvard Instruments, Boston, Mass.]) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Chol”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Currently, the most efficient and effective way to accomplish the transfer of genetic constructs into living cells is through the use of vector systems based on viruses that have been made replication defective. Some of the most effective vectors known in the art are those based on adeno-associated viruses (AAVs). AAVs are small viruses of the parvoviridae family that make attractive vectors for gene transfer in that they are replication defective, not known to cause any human disease, cause only a very mild immune response, can infect both actively dividing and quiescent cells, and stably persist in an extrachromosomal state without integrating into the target cell's genome. In certain embodiments, the present disclosure provides an AAV vector comprising the dCas9-based CRISPRi system of the disclosure.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northem blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Methods

In one aspect, the present disclosure includes a method of treating, ameliorating, and/or preventing progressive hearing loss in a subject need thereof. In certain embodiments, the method comprises administering to the subject an effective amount of an adeno-associated virus (AAV) vector comprising a nucleic acid encoding a superoxide dismutase (SOD) protein operably linked to a promoter, wherein the SOD protein is expressed in a cell of the inner ear thereby treating, ameliorating, and/or preventing the progressive hearing loss. In certain embodiments, the AAV vectors of the disclosure are targeted to the inner ear cell (e.g., hair cell). In certain embodiments, the AAV vector comprises a capsid protein derived from an AAV2. In certain embodiments, the capsid protein is AAV2-7m8. In certain embodiments, the AAV vector comprises a capsid protein derived from an AAV9. In certain embodiments, the AAV vector is administered via lumbar puncture, intravenous injection, nasal/olfactory delivery, pulmonary delivery, middle ear injection, round window diffusion, round window injection, oval window injection, intracochlear electrode or drug delivery system, and/or labyrinthotomy injection. In certain embodiments, the AAV vector is administered with an effective amount of an agent that disrupts the blood brain barrier.

In another aspect, the present disclosure includes a method of preventing, reversing, and/or minimizing progressive hearing loss in a subject need thereof. In certain embodiments, the method comprises administering to the subject an effective amount of an adeno-associated virus (AAV) vector comprising a nucleic acid encoding a superoxide dismutase (SOD) protein operably linked to a promoter, wherein the SOD protein is expressed in a cell of the inner ear thereby preventing, reversing, and/or minimizing the progressive hearing loss.

In certain embodiments, the inner ear cell is a hair cell, and expression of the SOD protein protects the cells from oxidative damage. In certain embodiments, the SOD protein is selected from the group consisting of SOD1, SOD2, and SOD3.

In certain embodiments, the cochlear cells are Deiter cells, Hansen cells, Claudius cells, primary afferent neurons, supporting cells, limbus cells, spiral ligament cells, and/or stria vascularis cells, and expression of the SOD protein protects the cells from oxidative damage. In certain embodiments, the vestibular cells are type I and type II vestibular hair cells of the vestibular end-organs, and expression of the SOD protein protects the cells from oxidative damage. In certain embodiments, the SOD protein is selected from the group consisting of SOD1, SOD2, and SOD3.

In certain embodiments, the genetic disease or disorder which is treated by way of the methods of the current disclosure is a disease or disorder associated with hearing loss. Non-limiting examples of hearing-loss diseases or disorders which could be treated by the methods of the disclosure include but are not limited to ototoxic drug-induced hearing loss (ODIHL), age-related hearing loss (ARHL), trauma-induced hearing loss, inflammatory- and autoimmune-induced hearing loss, and noise induced hearing loss (NIHL). Non-limiting examples of vestibular-loss diseases or disorders which could be treated by the methods of the disclosure include but are not limited to ototoxic drug-induced vestibular loss, age-related vestibular loss (presbystasis), inflammatory- and autoimmune-induced vestibular loss, and over-stimulation induced vestibular loss. Of these, noise-induced hearing loss is a major cause of hearing losses and is associated with overstimulation or physical damage of inner ear cells (e.g., inner and outer hair cells) that leads to oxidative damage and death of these cells. Ototoxic drug-induced hearing loss and vestibular loss can result, for example, as a side-effect of treatment with aminoglycoside antibiotics such as gentamicin, tobramycin, amikacin, plazomicin, streptomycin, neomycin, and paromomycin among others. ODIHL can also be an undesired side effect of anti-cancer treatment with certain platinum-based drugs such as cisplatin, carboplatin, and oxaliplatin among others. Age-related hearing loss and vestibular loss may result from a number of factors including certain environmental exposures, hereditary genetics, and other medical, disease-related, factors.

In certain embodiments, the AAV vector is administered with an effective amount of an agent that disrupts the blood brain barrier. Such agents are used to facilitate the access of therapeutic agents such as small molecules, viral vectors (e.g., AAV vectors), and other biologic molecules including proteins, antibodies, signaling molecules, and the like to the tissues of the central nervous system (CNS) including the inner ear, a space otherwise separated from the circulatory system by the blood brain barrier (BBB). The BBB comprises a semi-permeable membranous barrier located at the interface between the blood and the CNS tissue and composed of a complex system of endothelial cells, astroglia, pericytes, and perivascular mast cells. Reversible methods for permeabilizing the BBB sufficient to allow the AAV vectors of the disclosure access to the tissues of the inner ear include but are not limited to chemical methods using, for instance, mannitol treatment to induce osmotic changes that allow greater permeability of the BBB, physical methods using focused ultrasound, or engineering of the AAV capsid proteins to better diffuse across the BBB.

In another aspect, the present disclosure includes a method of reversing, minimizing, and/or preventing chemotherapy treatment-associated hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell of the subject thereby reversing, minimizing, and/or preventing the chemotherapy treatment-associated hearing loss in the subject.

In certain embodiments, the chemotherapy-induced hair cell loss is a platinum drug or platinum-based drug. Platinum drugs or platinum-based drugs are antineoplastic chemotherapeutic drugs that are commonly used to treat various kinds of cancers and include cisplatin, carboplatin, oxaliplatin, nedaplatin, lobaplatin, among others. Malignancies commonly treated with platinum drugs include cancers of the ovary, endometrium, bladder, testes, and head, and neck among others. They may also increase survival in some cases of squamous cell carcinoma and osteosarcoma. Platinum drugs form DNA adducts, which ultimately result in activation of p53-dependent and p-53 independent apoptotic pathways resulting in cell death. Relevant to the current disclosure, the cells of the cochlea are sensitive to platinum drug treatment with progressive and permanent hearing loss being a common and serious side effect of anti-cancer therapy with these drugs. In certain embodiments, the SOD protein expressing AAV vectors of the current disclosure are administered to a subject prior to treatment with one or more platinum drugs in order to counteract or prevent hearing loss. In certain embodiments, the platinum drug is cisplatin. It is also anticipated that the SOD protein expressing AAV vectors of the present disclosure could be used to treat chemotherapy-associated hearing loss that results from treatment with any platinum-based chemotherapeutic drug which results in increased oxidative damage in the cells of the inner ear.

In certain embodiments, the inner ear cell is a hair cell. In certain embodiments, the inner ear cell is a Deiter cell, Hansen cell, Claudius cell, primary afferent neuron, supporting cell, limbus cell, spiral ligament cell, and/or stria vascularis cell. In certain embodiments, the vestibular cell is a type I hair cell, type II hair cell, primary afferent neuron, and/or supporting cell. In certain embodiments, the expression of the SOD protein protects the cell from oxidative damage.

In certain embodiments, the AAV vector is targeted to the inner ear cell. In certain embodiments, the AAV vector comprises a capsid protein derived from an AAV2. In certain embodiments, the capsid protein is AAV2-7m8. In certain embodiments, the AAV vector comprises a capsid protein derived from an AAV9. In certain embodiments, the AAV vector is administered via middle ear injection, round window diffusion, round window injection, oval window injection, labyrinthotomy injection, intracochlear electrode or drug delivery system, oral, inhalation, nasal, nebulization, intravenous injection, intramuscular injection, intrathecal injection, intrapleural delivery, intracisterna magna injection, subcutaneous injection, and/or transdermal injection. In certain embodiments, the method further comprises administering to the subject an effective amount of an agent that disrupts the blood brain barrier. In certain embodiments, the SOD protein is selected from the group consisting of SOD1, SOD2, and SOD3.

### Pharmaceutical Compositions

Pharmaceutical compositions of the present disclosure may comprise as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione: adjuvants (e.g., aluminum hydroxide); and preservatives. Compositions of the present disclosure are preferably formulated for intravenous administration.

Pharmaceutical compositions of the present disclosure may comprise the AAV vector particles as described herein, in combination with one or more pharmaceutically or physiologically acceptable carriers, diluents or excipients. Such compositions may comprise buffers such as neutral buffered saline, phosphate-buffered saline (PBS) and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for a number of administration routes including middle ear injection, round window diffusion, round window injection, oval window injection, labyrinthomy injection, intracochlear electrode or drug delivery system, oral, inhalation, nasal, nebulization, intravenous injection, intramuscular injection, intrathecal injection, intrapleural injection, cisterna magna injection, subcutaneous injection, and/or transdermal injection. Pharmaceutical compositions of the present disclosure may be administered in a manner appropriate to the disease to be treated (or prevented). The quantity and frequency of administration will be determined by such factors as the condition of the patient, the type and severity of the patient's disease, and the type and functional nature of the patient's immune response to the phage particles, although appropriate dosages may be determined by clinical trials.

The AAV vector particles of the disclosure can be administered in dosages and routes and at times to be determined in appropriate pre-clinical and clinical experimentation and trials. Administration of the AAV vector particles of the disclosure may be combined with other methods useful to treat the desired disease or condition as determined by those of skill in the art.

In certain embodiments, the effective dose range is measured in units known to a person of skill in the art to be suitable for the description of AAV vector particle doses. In some embodiments, the effective dose range for a vaccine or therapeutic compound of the disclosure is measured by transducing units (TU)/kg/dose or genome copies (GC)/kg/dose or particles/kg/dose. In some embodiments, the dosage provided to a patient is between about 10⁶-10¹⁴ TU/kg. In some embodiments, the dosage provided to a patient is between about 10⁶-10¹⁴ GC/kg. In some embodiments, the effective dose range is measured by colony forming units (CFU), 50% tissue culture infectious dose (TCID₅₀), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

For humans, a medical doctor, e.g., physician or delegated advanced practice provider, having ordinary skill in the art can readily determine and prescribe the effective amount of the pharmaceutical composition required. For example, the physician or delegated advanced practice provider could start doses of the compounds of the disclosure employed in the pharmaceutical composition at levels lower than that required in order to achieve the desired therapeutic effect and gradually increase the dosage until the desired effect is achieved.

For animals, a medical doctor, e.g., physician or delegated advanced practice provider or veterinarian, having ordinary skill in the art can readily determine and prescribe the effective amount of the pharmaceutical composition required. For example, the physician or delegated advanced practice provider or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Dosage size can be adjusted according to the weight, age, and stage of the disease of the subject being treated. AAV vector particles may also be administered multiple times at these dosages. The AAV vector particles can be administered by using infusion techniques that are commonly known in the art of immunotherapy or vaccinology. The optimal dosage and treatment regime for a particular patient can readily be determined by one skilled in the art of medicine by monitoring the patient for signs of disease and adjusting the treatment accordingly.

The administration of the AAV vector particle compositions of the disclosure may be carried out in any convenient manner known to those of skill in the art. The AAV vector particles of the present disclosure may be administered to a subject by aerosol inhalation, injection, ingestion, transfusion, implantation or transplantation. The compositions described herein may be administered to a subject or patient via middle ear injection, round window diffusion, round window injection, oval window injection, labyrinthomy injection, intracochlear electrode or drug delivery system, oral, inhalation, nasal, nebulization, intravenous injection, intramuscular injection, intrathecal injection, intrapleural injection, cisterna magna injection, subcutaneous injection, and/or transdermal injection. It should be understood that the method and compositions that would be useful in the present disclosure are not limited to the particular formulations set forth in the examples.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and AAV particles of the disclosure, and, as such, may be considered in making and practicing the disclosure.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples, therefore, specifically point out the preferred embodiments of the present disclosure and are not to be construed as limiting in any way the remainder of the disclosure.

### Example 1: Expression of SOD1 Prior to Noise Exposure Preserves Hair Cell Propagation

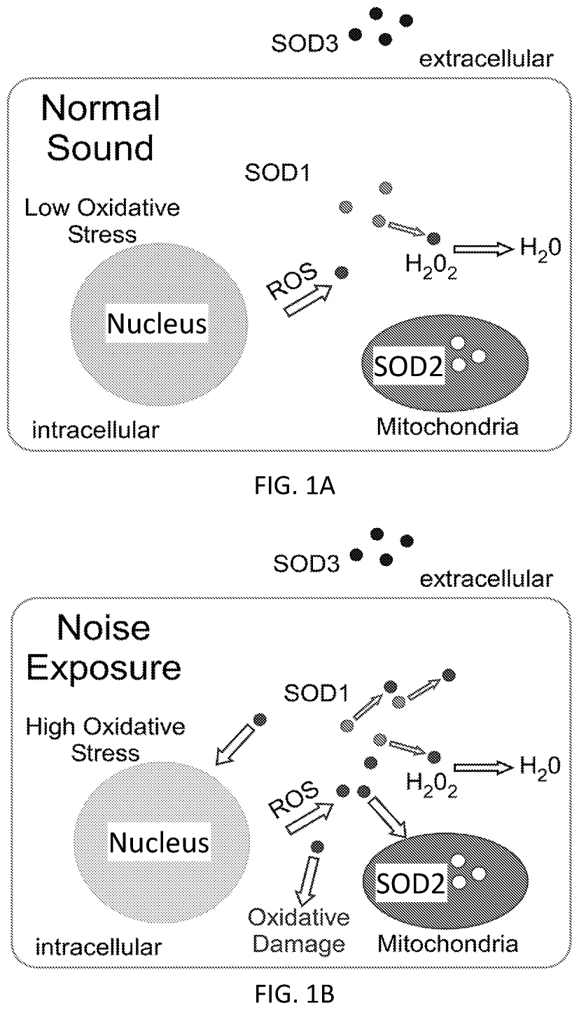

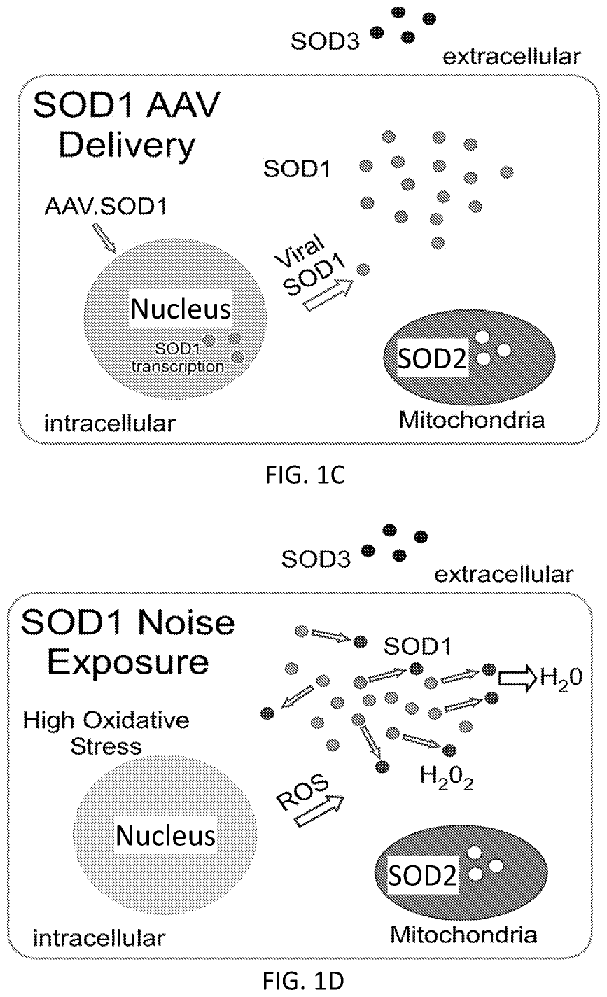

One primary cause for both progressive and noise-induced hearing loss involves hair cell death and damage due to oxidative stress. High levels of noise or long durations of exposure to medium levels of noise can lead to the overproduction of oxidative species known as free radicals. These are mostly composed of reactive oxygen species (ROS, e.g., peroxidases) that can damage various components within the cell, as well as the cell membrane. Hair cells have a natural protective enzyme against ROS called superoxidative dismutase (SOD). This comes in three versions, which operate in various regions of the cell. SOD1 operates internally, SOD2 operates within the mitochondria, and SOD3 operates extracellularly, but all three protect cellular component from oxidative damage under normal conditions (for example see FIG. 1A). In FIG. 1B, an example of how noise increases oxidative stress is shown. This leads to an increase in ROS that overwhelms the natural SOD1 levels of enzyme. The approach of the current disclosure uses AAV technology targeted to inner and outer hair cells to upregulate production of the SOD enzyme (here SOD1) to handle increased oxidative stress (FIG. 1B). FIG. 1D provides a hypothetical example of how this gene therapy functions in vivo during periods of noise exposure.

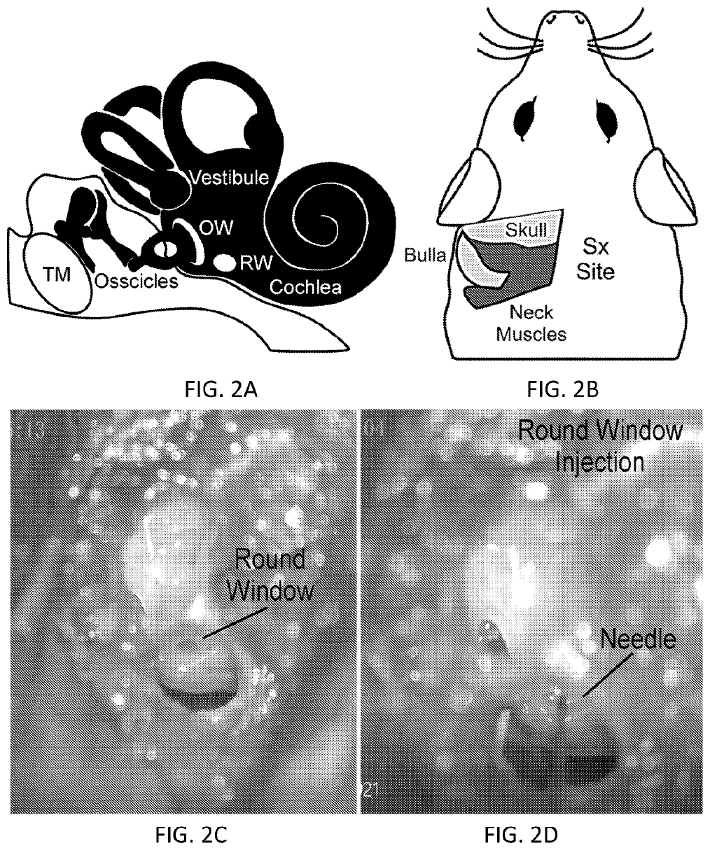

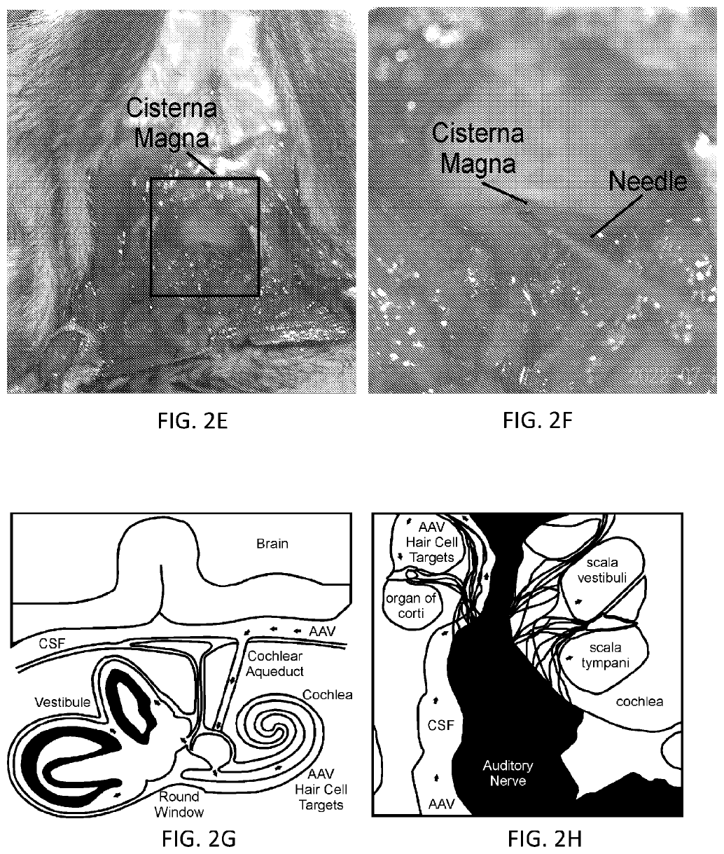

To deliver the AAV to its intended target in the inner ear (inner and outer hair cells) a promoter that is highly selective for these cell types will be used (e.g., 7m8). Two methods of delivery to the cerebral spinal fluid have been developed that achieve this goal (FIG. 2). In the gerbil, access to the round window of the cochlea allows direct injection of AAV into the inner ear (FIG. 2A). In humans this could also be accomplished through the middle ear. In gerbils, this is easily accessed through the bulla (FIG. 2B). Once the bulla over the cochlea has been removed (FIG. 2C) we can maneuver a gas tight Hamilton syringe to gently pierce the round window and then inject microliters of AAV (FIG. 2D). A second, and lower risk to the inner ear approach, involves injection through the cisterna magna (FIG. 2E). In humans this would be accomplish by delivery via a lumbar puncture (spinal tap). Once the cisterna magna is exposed, a small needle pierces the dura between the skull and the spinal cord allowing access to the cerebral spinal fluid (FIG. 2F). This does expose the greater brain to the AAV; however, using targeted AAV allows for the reduction or prevention of transfection of non-target cells. There are at least 2 pathways through which the AAV makes it to the inner ear from the CSF. In the first AAV enters the cochlea through the cochlear aqueduct (FIG. 2G). The second route of transfection occurs through the CSF that surrounds the auditory nerve where it has access to and innervates the organ of Corti, which contains the inner and outer hair cells as well as the primary afferent dendrites synapsing on the hair cells.

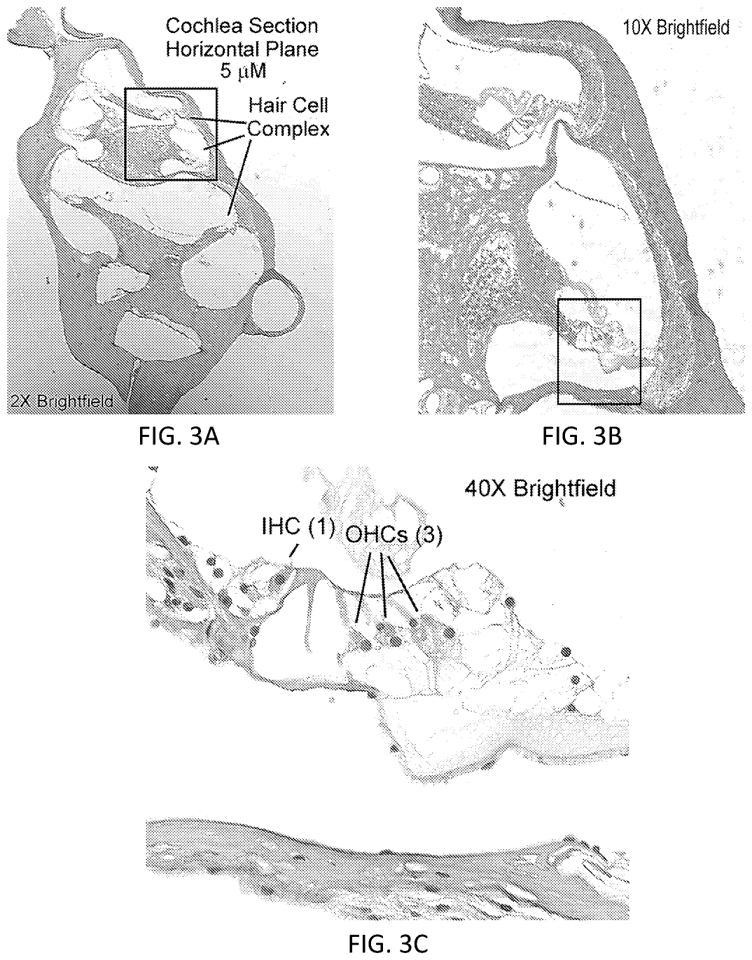

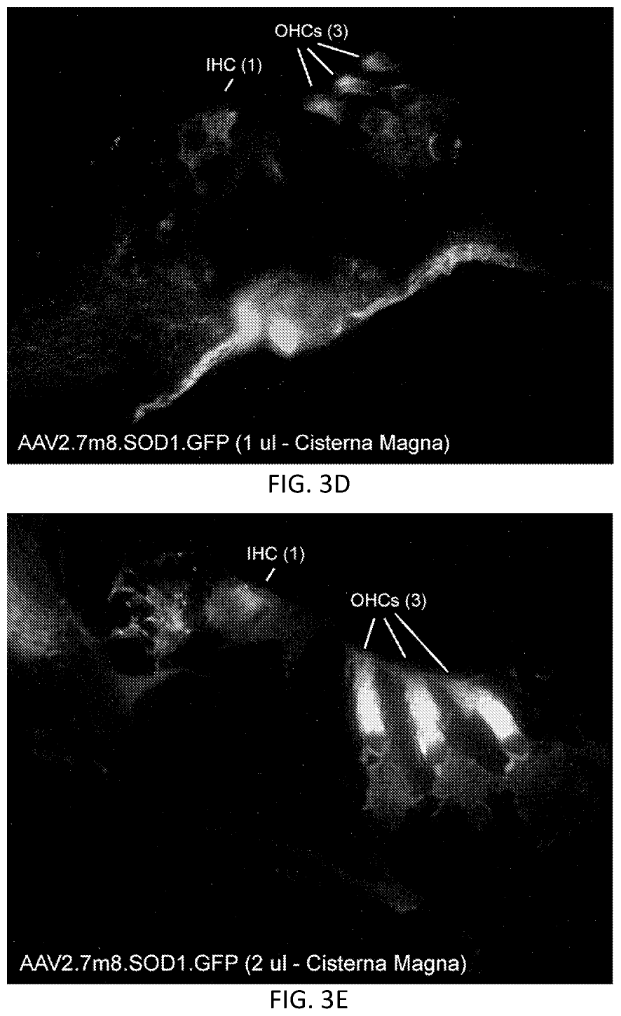

Regardless of the route of administration even small amounts of AAV (1 μL) will transfect both the inner and outer hair cells after cisterna magna injection (FIG. 3). In these proof of principle experiments a histological approach was used to horizontally section the cochlea (5 μm), which allowed for the visualization the organ of Corti containing the inner and outer hair cells respectively (FIG. 3A-C). Fluorescent imaging of these regions in animals that had been injected with the AAV.7m8.GFP two weeks prior showed good transfection into the inner and outer hair cells at both 1 μL and 2 μL doses of high titer AAV (10 to 13). The presence of GFP fluorescence confirmed successful transfection which enabled the progression to in vivo pilot studies.

In order to test the efficacy of the viral technology of the current invention, studies exposed animals to a noise exposure of 2 hours per day at 110 dB SPL, which has previously been shown to induce hair cell damage, neuropathy and synaptopathy of the cochlear inner and outer hair cells (Kujawa et al. (2009) J Neurosci. 29:14077-14085; Liberman MC. (2015) Sci Am. 313:48-53; Kurabi et al. (2017) Hear Res. 349:129-137).

Fourteen mongolian gerbils were used, which have human like audiograms, to test the hypothesis that upregulation of SOD1 in the inner and outer hair cells would prevent or reduce hair cell injury, neuropathy and synaptopathy. For this experiment animals received baseline ABR recordings and then were randomly selected to receive cisterna magna injections of physiological saline (N=8) or 3 μL of AAV.7m8.SOD1.GFP (N=6). Animals were then allowed 2 weeks for recovery and expression of the targeted AAV prior to the noise exposure. Animals were then exposed to 110 dB SPL noise for 2 hours a day for 5 days, and then returned to the home cage for 1 week followed by a terminal ABR recording and perfusion for histology.

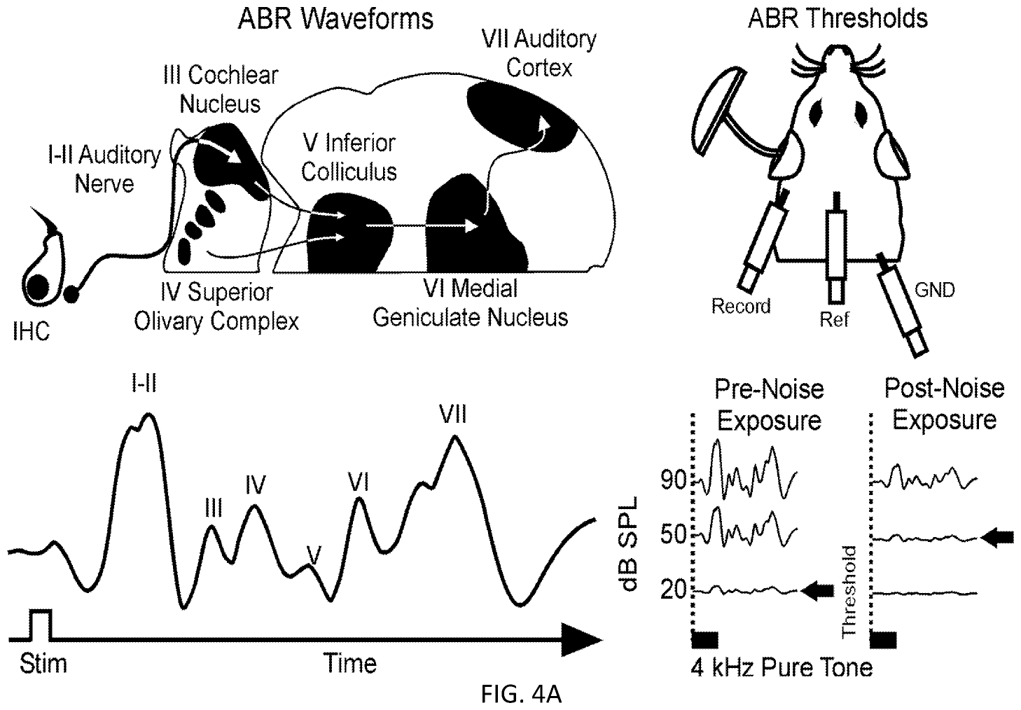

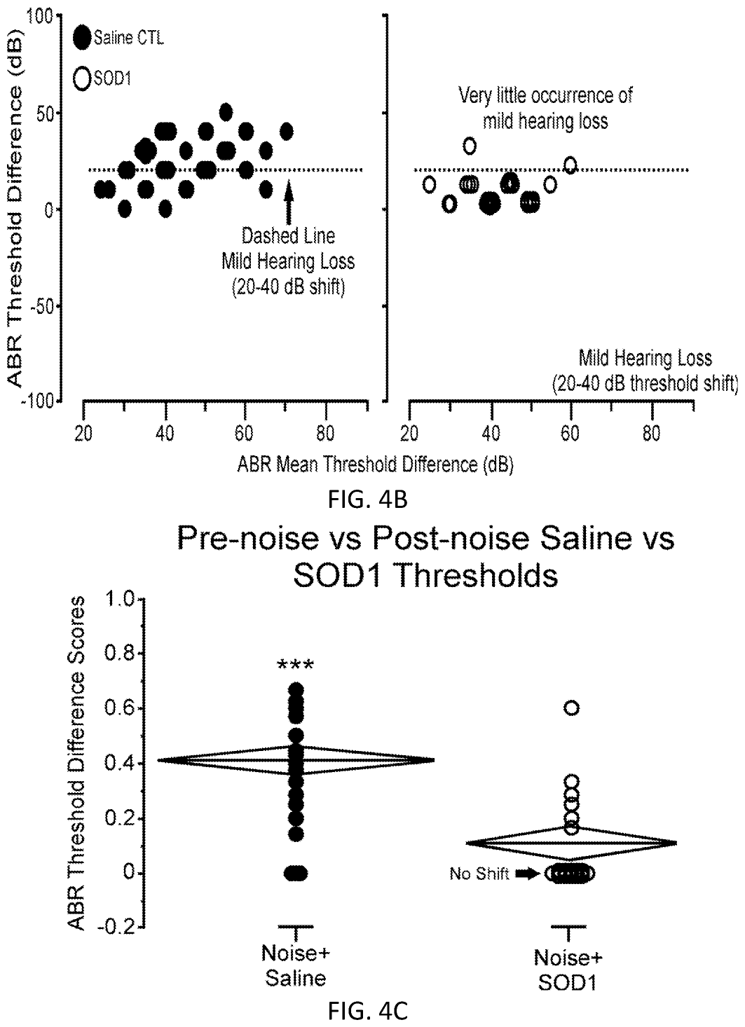

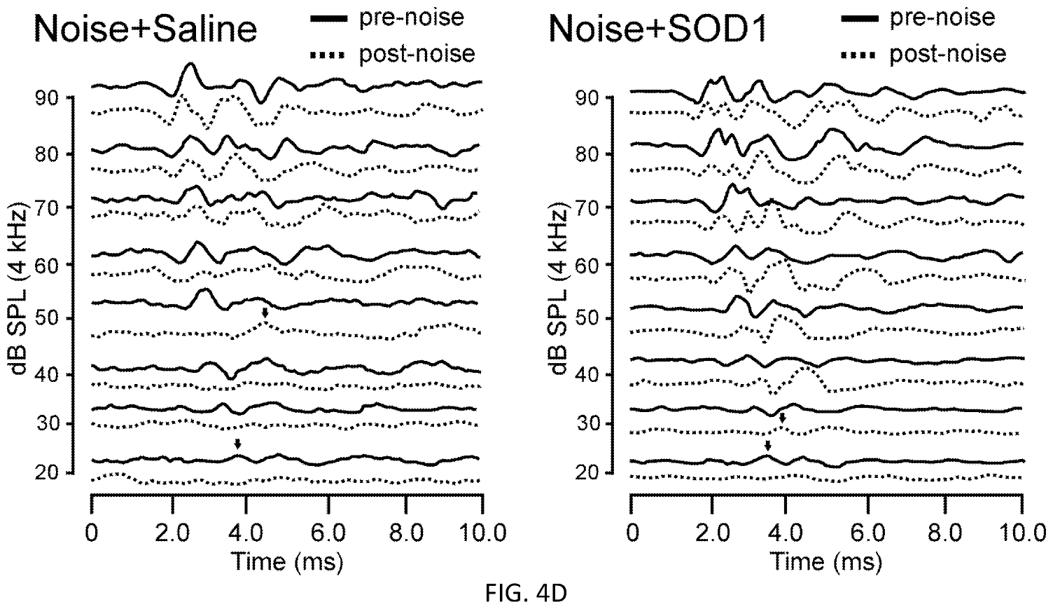

FIG. 4 shows the results for threshold measurements after noise exposure between both groups. In FIG. 4A a series of drawings depicts the ABR technique and how it corresponds to brain physiology. Wave 1 was measured (compound action potential generated at the distal portion of the cochlear nerve where it exits the cochlea) as well as amplitudes, latencies, and the overall threshold of hearing for each frequency (1, 2, 4, 8, or 16 kHz). FIG. 4B shows matched comparison test for the threshold difference scores. These scores represent the post-noise threshold value minus the pre-noise value, which shows an improvement (decrease) or worsening (increase) of hearing thresholds. These data demonstrated that, for the saline control group, a very significant worsening (increased) thresholds over pre-noise exposure, mostly above the mild hearing loss line (t=12.38, p<0.001). The group that received SOD1 had a small but significant worsening (increased) thresholds (t=4.1, p<0.05), with less hearing loss in the mild HL range. Direct comparison of the saline control vs SOD1 threshold difference scores in FIG. 4C shows a very significant worsening (increase) for the saline control group (Tukey's Honestly Significant Difference; q=1.99, p<0.0001). The ABR threshold is a good measure of neuropathy and IHC/OHC cell death); therefore, these results show that AAV.SOD1 expression had good protective effects during the noise exposure.

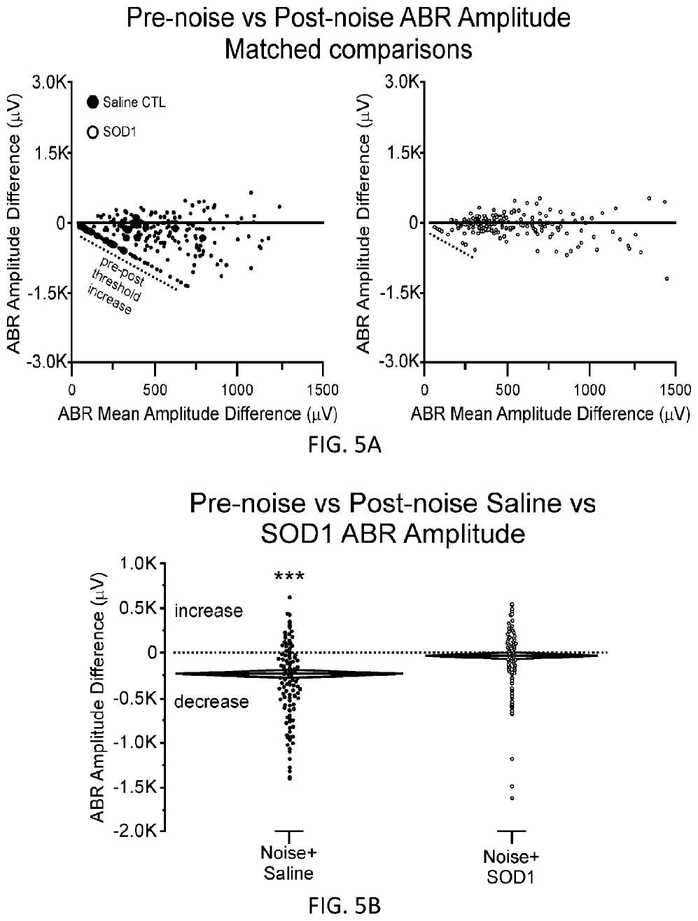

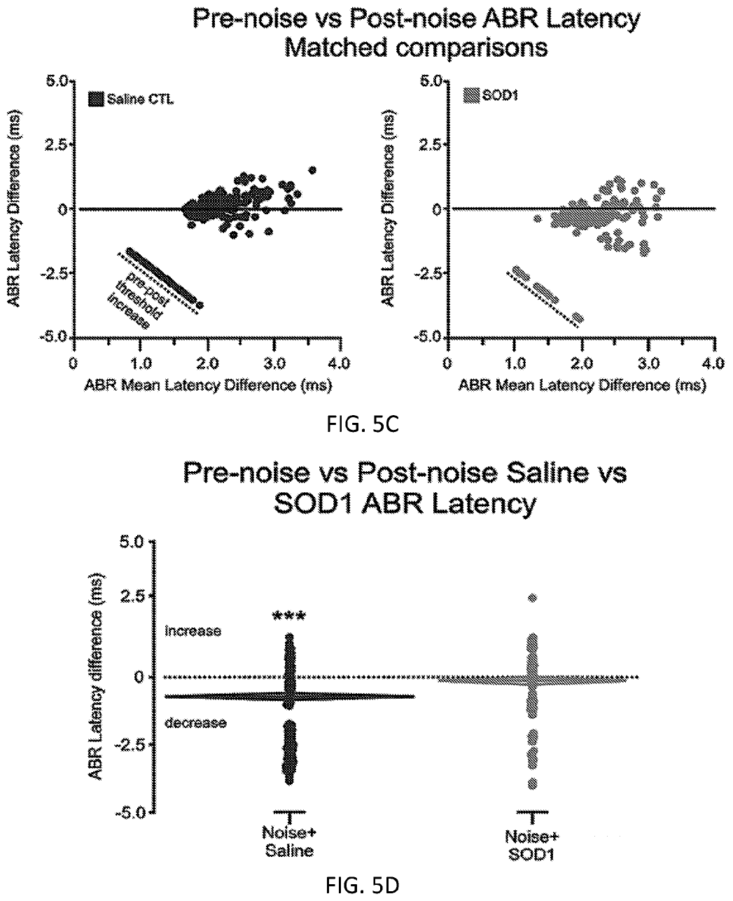

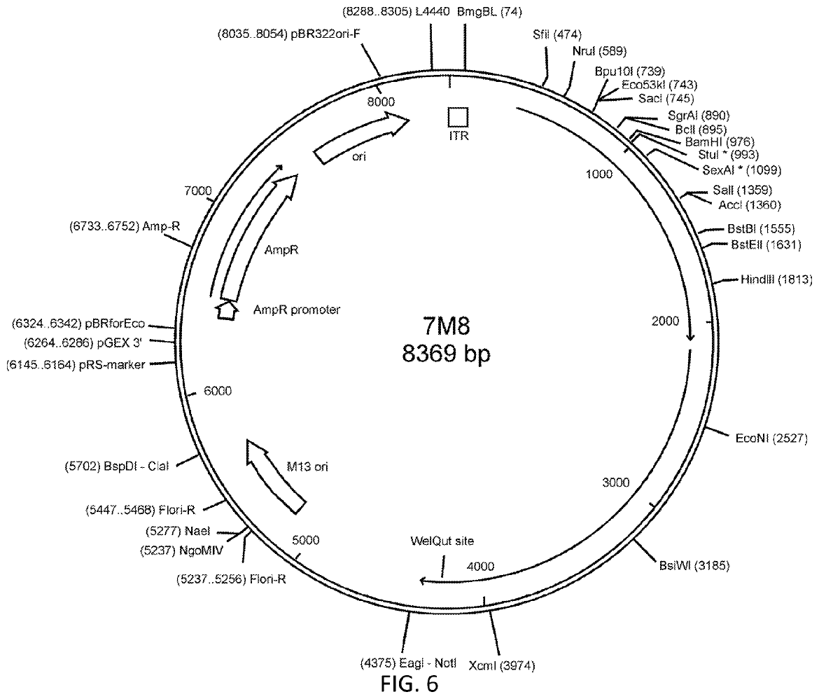

FIG. 5 shows the results for the ABR amplitude and latency measurements of wave 1 after noise exposure between both groups. FIG. 5A depicts a matched comparison test for amplitude difference scores. These scores represent the pre-noise amplitude minus the post-noise amplitude. These data demonstrate that the saline control group experienced a very significant decrease (worsening) in amplitudes from pre-noise exposure amplitudes (t=14.21, p<0.001). The group that received SOD1 had a small but significant decrease (worsening) in amplitudes (t=5.9, p<0.05). Direct comparison of the saline control vs SOD1 amplitude difference scores in FIG. 5B shows a very significant decrease (worsening) in amplitude for the saline control group compared to the SOD1 group (Tukey's Honestly Significant Difference: q=1.96, p<0.0001). In FIG. 5C a matched comparison test is shown for the ABR latency difference scores (post-noise latency minus pre-noise latency). Here you can see that for the saline control group a very significant increase (worsening) in ABR latency from pre-noise exposure amplitudes (t=10.11, p<0.001). The group that received SOD1 had no significant increase (worsening) in ABR latency (t=1.4, p=0.22). Direct comparison of the saline control vs SOD1 latency difference scores in FIG. 5D shows a very significant increase (worsening) in ABR latency for the saline control group compared to the SOD1 group (Tukey's Honestly Significant Difference; q=1.96, p<0.0001). Decreases (worsening) in ABR amplitude measurements and increased ABR latency measurements are indicators of auditory hair cell and auditory nerve fiber synaptopathy. Therefore, these results show that AAV.SOD1 expression protected and minimized auditory hair cell and fiber synapse loss during noise exposure.

### Example 2: Clinical Development of an AAV-Directed Antioxidant Therapy for Hearing Loss

In certain embodiments, the gene therapy of the present disclosure comprises the following sequenced parts: AAV2-7m8.CAG.SOD1.IRES.eGFP.WPRE.SV40. Here AAV2.7m8 is the serotype in which a 7mer insertion occurs in the AAV2 cap. This AAV serotype has a high rate of transfection in inner and outer hair cells of the cochlea, as well as type I and type II hair cells in the vestibular end-organs. The CAG promoter is a strong synthetic promoter frequently used to drive high levels of gene expression in mammalian expression vectors. The SOD1 sequence is the gerbil specific nucleotide sequence for superoxide dismutase 1. IRES (internal ribosome entry site), is an RNA element that allows for translation initiation in a cap-independent manner, as part of the greater process of protein synthesis allowing for the co-expression of several genes under the control of the same promoter. eGFP or green fluorescent protein (GFP) is a protein that exhibits bright green fluorescence when exposed to light in the blue to ultraviolet range and facilitates researchers in identifying transfected cells. WPRE (Woodchuck Hepatitis Virus (WHP) Posttranscriptional Regulatory Element (WPRE)) is a DNA sequence that enhances the expression of genes delivered by viral vectors. SV40 is an abbreviation for simian vacuolating virus 40 or simian virus 40 that enhances gene expression.

It is important to note that while this constructed AAV has shown significant efficacy in transfection and treatment in gerbil models, the sequence of a successful human variant with FDA approval of the AAV would be different in many possible ways. First, the human sequence for SOD enzymes is different from the Mongolian gerbil. Consequently, in certain embodiments, an AAV constructed for use in non-human primates will comprise a nucleotide sequence specific to that species (e.g., macaque). Thus, the SOD nucleotide sequences used in certain embodiments is matched to the species of the subject receiving the treatment. In certain embodiments, the invention of the present disclosure comprises a human variant appropriate for clinical use. In certain embodiments, the studies disclosed in this example comprise SOD1 sequences, as well as SOD2, and SOD3 variants 1 and 2. It is to be understood that, in certain embodiments, the isoform of SOD gene used in the invention could be one or all of these sequences. Additionally, there are many different serotypes that have shown efficacious transfection of cochlear hair cells including AAV1.RPG, AAV2, AAV2.HDG, AAV2.7m8, AAV8, AAV8.BP2, AAV.Anc80.L65, AAV9, AAV9.KGG among others. In certain embodiments, the CAG promotor is one of many types of promoters that can be used in AAV construction. The skilled artisan would be able to select a promoter appropriate for use in the desired subject, including non-human primate and humans according to safety and efficacy. Thus, in certain embodiments, a different promoter that expresses in human cochlear tissue at higher levels would be used. In certain embodiments, the therapeutic AAV of the current invention will not comprise GFP. Along these lines, the IRES sequence, which is used to enhance expression of multiple transgenes (SOD1,GFP) might not be necessary for clinically useful embodiments of the current invention since only a single transgene is present. Additionally, similar to promoters, there are many types of enhancers that could be selected for use in certain embodiments of the invention.

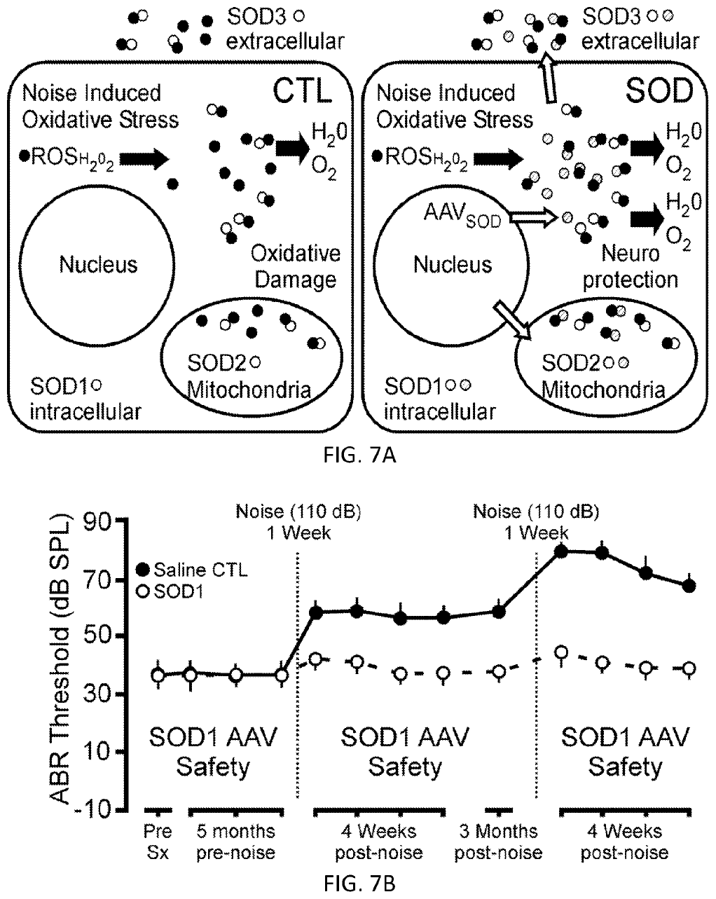

FIG. 7A depicts a diagram showing the normal effect of noise induced oxidative stress on a normal cell (left) and a cell having received SOD gene therapy (right). Increased bioavailability of SOD provides neuroprotection from free radicals over produced by noise exposure. An animal study examining the effect of SOD1 AAV treatment on moderately high noise exposure over a long period of time was then conducted. Here, a group of animals (N8) received SOD1 injections (5 ml) followed by 5 months of monitoring for auditory health (FIG. 7B). There were no changes from baseline. In the next phase SOD1 and control animals were exposed to 110 dB for 2 hours per day for 5 days. This was followed by 3 months of post exposure monitoring. Results demonstrated a significant neuroprotection in the SOD1 gene therapy group and that CTL animals developed persistent moderate hearing loss. Finally, animals were then exposed to a second round of noise (110 dB SPL, 2 hours/day over 5 days). Again, there was a persistent neuroprotective effect of the SOD1 gene therapy, while the control animals developed severe hearing loss.

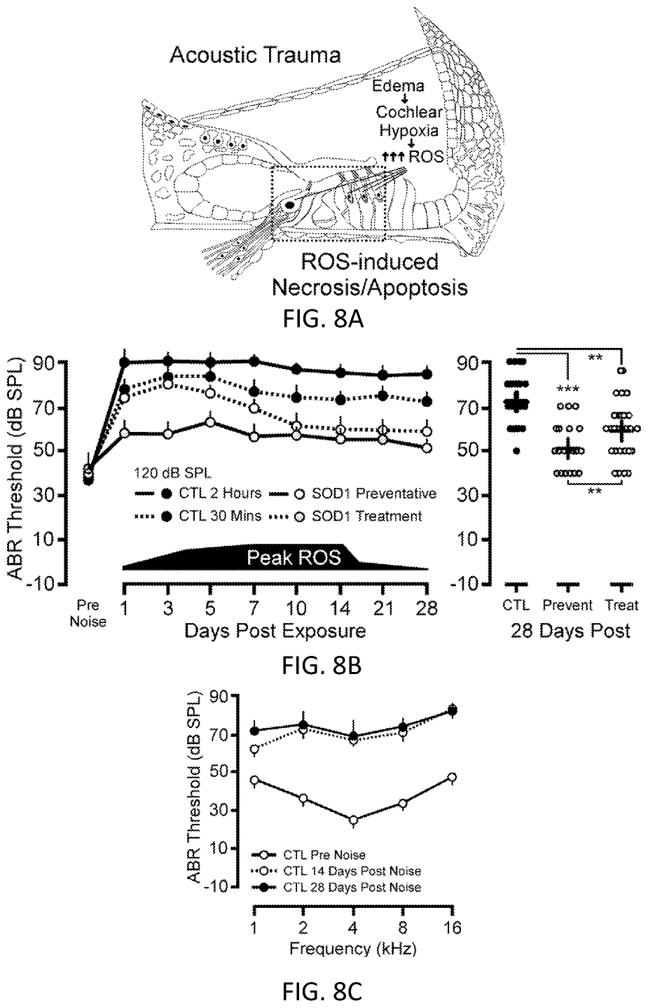

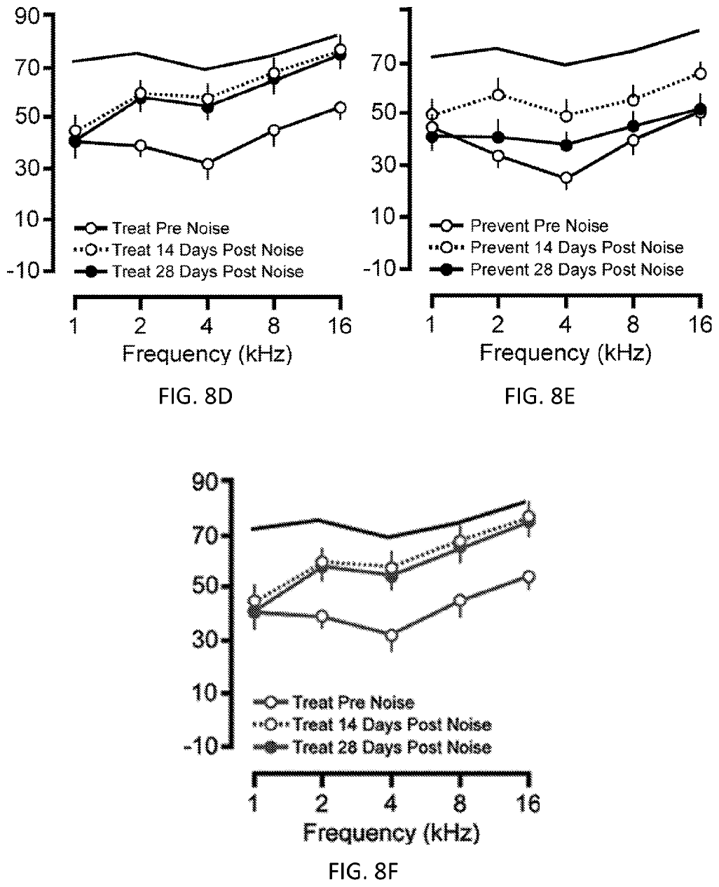

A series of studies was then conducted to examine the effect AAV-SOD treatment of hearing damage caused by short-term exposure to high levels of noise. FIG. 8A is a diagram depicting the effect of acoustic trauma (120 dB, 30 minutes) on the cochlear complex of the inner ear. Here, control animals were exposed to 120 dB SPL noise for 30 minutes, t hour, and 2 hours. These animals developed significant persistent hearing loss over 4 weeks. Animals that received SOD1 treatment prior to exposure had significant neuroprotection over time. Animals that received SOD1 treatment after noise exposure (1 hour post) were significantly protected from hearing loss which corresponded to the expression of SOD1 transgene over time (˜2 weeks to peak). See FIGS. 8B-8F.

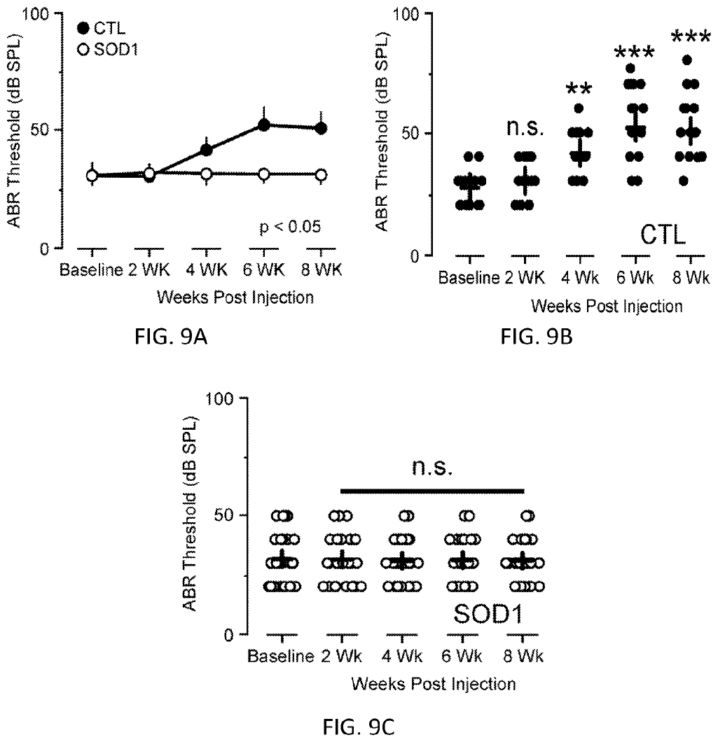

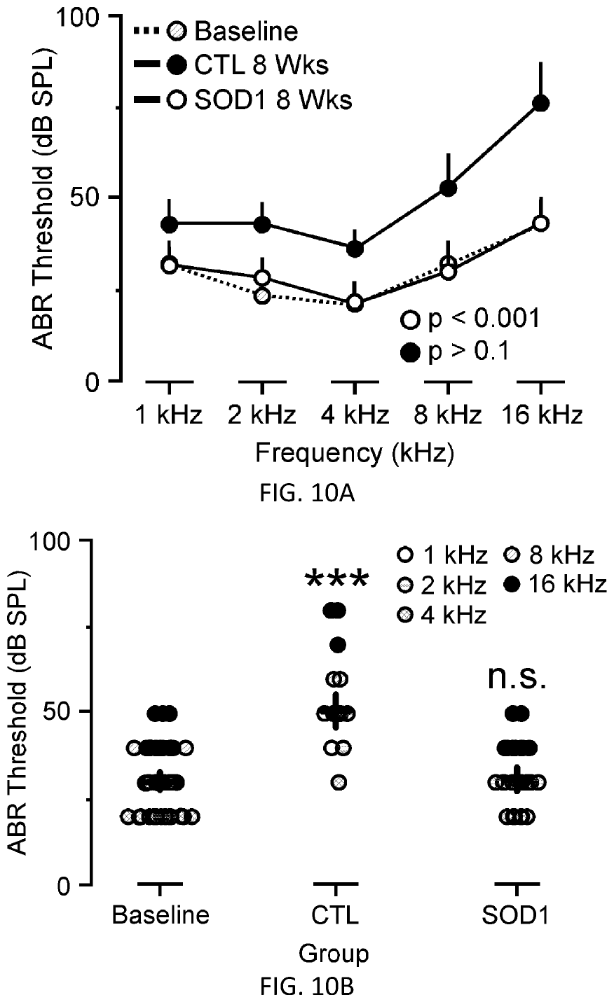

Cisplatin is a platinum-based chemotherapy drug and is commonly used in about 20% of US cancer patients for the treatment of many cancers, including bladder, ovarian, and testicular cancers. A common side effect of cisplatin treatment is progressive hearing loss resulting from damage to the cochlea, leaving 40%-80% of adults, and at least 50% of children, with significant permanent hearing loss, a condition that can greatly affect quality of life. In order to see if treatment with AAV SOD therapy could prevent cisplatin-related hearing loss, a study was conducted in which animals were treated with weekly doses of 2 mg/kg cisplatin i.p. for 8 weeks following administration of AAV SOD treatment or a vehicle control. Results from these studies are illustrated by FIGS. 9A-9C. Control animals experienced progressive hearing loss across all frequencies while SOD1 treated animals were consistently protected. Further analysis of these data in FIGS. 10A and 10B found that SOD1 pretreatment produced nearly 100% neuroprotection across frequency ranges. Without wishing to be bound by theory, these data suggest that AAV SOD therapy could be useful in protecting patients from this common side effect of cisplatin treatment.

### ENUMERATED EMBODIMENTS

The following enumerated embodiments are provided, the numbering of which is not to be construed as designating levels of importance.

Embodiment 1 provides a polynucleotide encoding an adeno-associated virus (AAV) vector. In certain embodiments, the AAV vector comprises a first AAV inverted terminal repeat (ITR) nucleic acid sequence. In certain embodiments, the AAV vector comprises a nucleic acid encoding a superoxide dismutase (SOD) protein operably linked to a promoter. In certain embodiments, the AAV vector comprises a second AAV inverted terminal repeat (ITR) nucleic acid sequence. In certain embodiments, the AAV vector is targeted to a cell of the inner ear.

Embodiment 2 provides the polynucleotide of embodiment 1, wherein the AAV vector further comprises at least one AAV capsid protein.

Embodiment 3 provides the polynucleotide of embodiment 2, wherein the capsid protein is AAV2.

Embodiment 4 provides the polynucleotide of embodiment 3, wherein the capsid protein is AAV2-7M8.

Embodiment 5 provides the polynucleotide of embodiment 2, wherein the capsid protein is AAV9.

Embodiment 6 provides the polynucleotide of any one of embodiments 1-5, wherein the inner ear cell is a hair cell.

Embodiment 7 provides the polynucleotide of any one of embodiments 1-6, wherein the inner ear cell is a Deiter cell, Hansen cell, Claudius cell, primary afferent neuron, supporting cell, limbus cell, spiral ligament cell, stria vascularis cell, type I vestibular hair cell, and/or type II vestibular hair cell.

Embodiment 8 provides the polynucleotide of any one of embodiments 1-7, wherein the vector expresses the SOD protein in the inner ear cell.

Embodiment 9 provides the polynucleotide of any one of embodiments 1-8, wherein the SOD protein is selected from the group consisting of SOD1, SOD2, and SOD3.

Embodiment 10 provides a recombinant adeno-associated virus (AAV) vector. In certain embodiments, the AAV vector comprises a first AAV inverted terminal repeat (ITR) nucleic acid sequence. In certain embodiments, the AAV vector comprises a second AAV inverted terminal repeat (ITR) nucleic acid sequence. In certain embodiments, the AAV vector comprises a polynucleotide sequence encoding a superoxide dismutase (SOD) protein operably linked to a promoter flanked by the first AAV inverted terminal repeat (ITR) sequence and the second AAV ITR. In certain embodiments, the AAV vector has a specificity for a cell of the inner ear.

Embodiment 11 provides the recombinant AAV of embodiment 10, wherein the AAV vector further comprises at least one AAV capsid protein.

Embodiment 12 provides the recombinant AAV of embodiment 11, wherein the at least one AAV capsid protein is AAV2 or a derivative thereof.

Embodiment 13 provides the recombinant AAV of any one of embodiments 11-12, wherein the at least one capsid protein is AAV2-7M8.

Embodiment 14 provides the recombinant AAV of embodiment 11, wherein the at least one AAV capsid protein is AAV9 or a derivative thereof.

Embodiment 15 provides the recombinant AAV of any one of embodiments 10-14, wherein the AAV expresses the SOD protein in the inner ear cell.

Embodiment 16 provides the recombinant AAV of any one of embodiments 10-15, wherein the inner ear cell is a hair cell.

Embodiment 17 provides the recombinant AAV of any one of embodiments 10-16, wherein the inner ear cell is a Deiter cell, Hansen cell, Claudius cell, primary afferent neuron, supporting cell, limbus cell, spiral ligament cell, type I vestibular hair cell, and/or type II vestibular hair cell.

Embodiment 18 provides the recombinant AAV of any one of embodiments 10-17, wherein the SOD protein is selected from the group consisting of SOD1, SOD2, and SOD3.

Embodiment 19 provides a cell comprising an adeno-associated virus (AAV) vector comprising a first AAV inverted terminal repeat (ITR) nucleic acid sequence, a second AAV inverted terminal repeat (ITR) nucleic acid sequence, a nucleic acid encoding a superoxide dismutase (SOD) protein operably linked to a promoter flanked by the first AAV inverted terminal repeat (ITR) sequence and the second AAV ITR.

Embodiment 20 provides the cell of embodiment 19, wherein the cell is an inner ear cell.

Embodiment 21 provides the cell of any one of embodiments 19-20, wherein the cell is a hair cell.

Embodiment 22 provides the cell of any one of embodiments 19-21, wherein the cell is a Deiter cell, Hansen cell, Claudius cell, primary afferent neuron, supporting cell, limbus cell, spiral ligament cell, type I vestibular hair cell, and/or type II vestibular hair cell.

Embodiment 23 provides a method of treating, ameliorating, and/or preventing progressive hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell, thereby treating, ameliorating, and/or preventing the progressive hearing loss in the subject.

Embodiment 24 provides the method of embodiment 23, wherein the inner ear cell is a hair cell.

Embodiment 25 provides the method of any one of embodiments 23-24, wherein the inner ear cell is a Deiter cell, Hansen cell, Claudius cell, primary afferent neuron, supporting cell, limbus cell, spiral ligament cell, type I vestibular hair cell, and/or type II vestibular hair cell.

Embodiment 26 provides the method of any one of embodiments 23-25, wherein expression of the SOD protein protects the cell from oxidative damage.

Embodiment 27 provides the method of any one of embodiments 23-26, wherein the AAV vector is targeted to the inner ear cell.

Embodiment 28 provides the method of any one of embodiments 23-27, wherein the AAV vector comprises a capsid protein derived from an AAV2.

Embodiment 29 provides the method of embodiment 28, wherein the capsid protein is AAV2-7m8.

Embodiment 30 provides the method of any one of embodiments 23-27, wherein the AAV vector comprises a capsid protein derived from an AAV9.

Embodiment 31 provides the method of any one of embodiments 23-30, wherein the AAV vector is administered via middle ear injection, round window diffusion, round window injection, oval window injection, labyrinthotomy injection, intracochlear electrode or drug delivery system, oral, inhalation, nasal, nebulization, intravenous injection, intramuscular injection, intrathecal injection, intrapleural injection, cisterna magna injection, subcutaneous injection, and/or transdermal injection.

Embodiment 32 provides the method of any one of embodiments 23-31, further comprising administering the AAV vector with an effective amount of an agent that disrupts the blood brain barrier.

Embodiment 33 provides the method of any one of embodiments 23-32, wherein the SOD protein is selected from the group consisting of SOD1, SOD2, and SOD3.

Embodiment 34 provides a method of reversing, minimizing, and/or preventing progressive hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell of the subject thereby reversing, minimizing, and/or preventing the progressive hearing loss in the subject.

Embodiment 35 provides the method of embodiment 34, wherein the inner ear cell is a hair cell.

Embodiment 36 provides the method of any one of embodiments 34-35, wherein the inner ear cell is a Deiter cell. Hansen cell, Claudius cell, primary afferent neuron, supporting cell, limbus cell, spiral ligament cell, type I vestibular hair cell, and/or type II vestibular hair cell.

Embodiment 37 provides the method of any one of embodiments 34-36, wherein expression of the SOD protein protects the cell from oxidative damage.

Embodiment 38 provides the method of any one of embodiments 34-37, wherein the AAV vector is targeted to the inner ear cell.

Embodiment 39 provides the method of any one of embodiments 34-38, wherein the AAV vector comprises a capsid protein derived from an AAV2.

Embodiment 40 provides the method of embodiment 39, wherein the capsid protein is AAV2-7m8.

Embodiment 41 provides the method of any one of embodiments 34-38, wherein the AAV vector comprises a capsid protein derived from an AAV9.

Embodiment 42 provides the method of any one of embodiments 34-41, wherein the AAV vector is administered via middle ear injection, round window diffusion, round window injection, oval window injection, labyrinthotomy injection, intracochlear electrode or drug delivery system, oral, inhalation, nasal, nebulization, intravenous injection, intramuscular injection, intrathecal injection, intrapleural injection, cisterna magna injection, subcutaneous injection, and/or transdermal injection.

Embodiment 43 provides the method of any one of embodiments 34-42, further comprising administering to the subject an effective amount of an agent that disrupts the blood brain barrier.

Embodiment 44 provides the method of any one of embodiments 34-43, wherein the SOD protein is selected from the group consisting of SOD1, SOD2, and SOD3.

Embodiment 45 provides a method of reversing, minimizing, and/or preventing chemotherapy treatment-associated hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell of the subject thereby reversing, minimizing, and/or preventing the chemotherapy treatment-associated hearing loss in the subject.

Embodiment 46 provides the method of embodiment 45, wherein the chemotherapy is a platinum drug.

Embodiment 47 provides the method of embodiment 46, wherein the platinum drug is cisplatin.

Embodiment 48 provides the method of any one of embodiments 45-47, wherein the inner ear cell is a hair cell.

Embodiment 49 provides the method of any one of embodiments 45-48, wherein the inner ear cell is a Deiter cell. Hansen cell, Claudius cell, primary afferent neuron, supporting cell, limbus cell, spiral ligament cell, type I vestibular hair cell, and/or type II vestibular hair cell.

Embodiment 50 provides the method of any one of embodiments 45-49, wherein expression of the SOD protein protects the cell from oxidative damage.

Embodiment 51 provides the method of any one of embodiments 45-50, wherein the AAV vector is targeted to the inner ear cell.

Embodiment 52 provides the method of any one of embodiments 45-51, wherein the AAV vector comprises a capsid protein derived from an AAV2.

Embodiment 53 provides the method of embodiment 52, wherein the capsid protein is AAV2-7m8.

Embodiment 54 provides the method of any one of embodiments 45-51, wherein the AAV vector comprises a capsid protein derived from an AAV9.

Embodiment 55 provides the method of any one of embodiments 45-54, wherein the AAV vector is administered via middle ear injection, round window diffusion, round window injection, oval window injection, labyrinthotomy injection, intracochlear electrode or drug delivery system, oral, inhalation, nasal, nebulization, intravenous injection, intramuscular injection, intrathecal injection, intrapleural delivery, cisterna magna injection, subcutaneous injection, and/or transdermal injection.

Embodiment 56 provides the method of any one of embodiments 45-55, further comprising administering to the subject an effective amount of an agent that disrupts the blood brain barrier.

Embodiment 57 provides the method of any one of embodiments 45-56, wherein the SOD protein is selected from the group consisting of SOD1, SOD2, and SOD3.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A polynucleotide encoding an adeno-associated virus (AAV) vector comprising:
a first AAV inverted terminal repeat (ITR) nucleic acid sequence,
a nucleic acid encoding a superoxide dismutase (SOD) protein operably linked to a promoter, and
a second AAV inverted terminal repeat (ITR) nucleic acid sequence,
wherein the vector is targeted to a cell of the inner ear.

2. The polynucleotide of claim 1, wherein the AAV vector further comprises at least one AAV capsid protein.

3. The polynucleotide of claim 2, wherein the capsid protein is AAV2 or AAV9, optionally wherein the capsid protein is AAV2-7M8.

4-5. (canceled)

6. The polynucleotide of claim 1, wherein the inner ear cell is a hair cell, optionally wherein the inner ear cell is at least one of Deiter cell, Hansen cell, Claudius cell, primary afferent neuron, supporting cell, limbus cell, spiral ligament cell, type I vestibular hair cell, and type II vestibular hair cell.

7. (canceled)

8. The polynucleotide of claim 1, wherein the vector expresses the SOD protein in the inner ear cell, optionally wherein the SOD protein is selected from the group consisting of SOD1, SOD2, and SOD3.

9. (canceled)

10. A recombinant adeno-associated virus (AAV) vector comprising:
a first AAV inverted terminal repeat (ITR) nucleic acid sequence,
a second AAV inverted terminal repeat (ITR) nucleic acid sequence, and
a polynucleotide sequence encoding a superoxide dismutase (SOD) protein operably linked to a promoter flanked by the first AAV inverted terminal repeat (ITR) sequence and the second AAV ITR,
wherein the AAV vector has a specificity for a cell of the inner ear.

11. The recombinant AAV of claim 10, wherein the AAV vector further comprises at least one AAV capsid protein.

12. The recombinant AAV of claim 11, wherein at least one AAV capsid protein is AAV2, AAV9, or a derivative thereof, optionally wherein the capsid protein is AAV2-7M8.

13-14. (canceled)

15. The recombinant AAV of claim 10, wherein the AAV expresses the SOD protein in the inner ear cell, optionally wherein the SOD protein is selected from the group consisting of SOD1, SOD2, and SOD3.

16. The recombinant AAV of claim 15, wherein the inner ear cell is a hair cell, optionally wherein the inner ear cell is at least one of a Deiter cell, Hansen cell, Claudius cell, primary afferent neuron, supporting cell, limbus cell, spiral ligament cell, type I vestibular hair cell, and type II vestibular hair cell.

17-18. (canceled)

19. A cell comprising an adeno-associated virus (AAV) vector comprising:
a first AAV inverted terminal repeat (ITR) nucleic acid sequence,
a second AAV inverted terminal repeat (ITR) nucleic acid sequence, and
a nucleic acid encoding a superoxide dismutase (SOD) protein operably linked to a promoter flanked by the first AAV inverted terminal repeat (ITR) sequence and the second AAV ITR.

20. The cell of claim 19, wherein the cell is an inner ear cell.

21. The cell of claim 20, wherein the cell is a hair cell, optionally wherein the cell is at least one of a Deiter cell, Hansen cell, Claudius cell, primary afferent neuron supporting cell, limbus cell, spiral ligament cell, type I vestibular hair cell, and type II vestibular hair cell.

22. (canceled)

23. A method of treating, ameliorating, or preventing progressive hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell, thereby treating, ameliorating, or preventing the progressive hearing loss in the subject.

24. The method of claim 23, wherein the inner ear cell is a hair cell, optionally wherein the inner ear cell is at least one of a Deiter cell, Hansen cell, Claudius cell prima afferent neuron, supporting cell, limbus cell, spiral ligament cell, type I vestibular hair cell, and type II vestibular hair cell.

25. (canceled)

26. The method of claim 23, wherein at least one of the following applies:
(a) expression of the SOD protein protects the cell from oxidative damage;
(b) the AAV vector is targeted to the inner ear cell;
(c) the AAV vector comprises a capsid protein derived from an AAV2, optionally wherein the capsid protein is AAV2-7m8;
(d) the AAV vector comprises a capsid protein derived from an AAV9;
(e) the AAV vector is administered via middle ear injection, round window diffusion, round window injection, oval window injection, labyrinthotomy injection, intracochlear electrode or drug delivery system, oral, inhalation, nasal, nebulization, intravenous injection, intramuscular injection, intrathecal injection, intrapleural delivery, cisterna magna injection, subcutaneous injection, or transdermal injection;
(f) the method further comprises administering an effective amount of an agent that disrupts the blood brain barrier;
(g) the SOD protein is selected from the group consisting of SOD1, SOD2, and SOD3.

27-33. (canceled)

34. A method of reversing, minimizing, or preventing progressive hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell of the subject thereby reversing, minimizing, or preventing the progressive hearing loss in the subject.

35. The method of claim 34, wherein at least one of the following applies:
(a) the inner ear cell is a hair cell optionally wherein the inner ear cell is at least one of a Deiter cell, Hansen cell, Claudius cell, primary afferent neuron, supporting cell, limbus cell, spiral ligament cell, type I vestibular hair cell, and type II vestibular hair cell;
(b) expression of the SOD protein protects the cell from oxidative damage;
(c) the AAV vector is targeted to the inner ear cell;
(d) the AAV vector comprises a capsid protein derived from an AAV2, optionally wherein the capsid protein is AAV2-7m8;
(e) the AAV vector comprises a capsid protein derived from an AAV9;
(f) the AAV vector is administered via middle ear injection, round window diffusion, round window injection, oval window injection, labyrinthotomy injection·intracochlear electrode or drug delivery system, oral, inhalation, nasal, nebulization, intravenous injection, intramuscular injection, intrathecal injection, intrapleural delivery, cisterna magna injection, subcutaneous injection, or transdermal injection;
(g) the method further comprises administering to the subject an effective amount of an agent that disrupts the blood brain barrier;
(h) the SOD protein is selected from the group consisting of SOD1, SOD2, and SOD3.

36-44. (canceled)

45. A method of reversing, minimizing, or preventing chemotherapy treatment-associated hearing loss in a subject need thereof, the method comprising administering to the subject an effective amount of an adeno-associated virus (AAV) vector comprising a nucleic acid encoding a superoxide dismutase (SOD) protein operably linked to a promoter, wherein the SOD protein is expressed in an inner ear cell of the subject thereby reversing, minimizing, or preventing the chemotherapy treatment-associated hearing loss in the subject.

46. The method of claim 45, wherein at least one of the following applies:
(a) the chemotherapy is a platinum drug, optionally wherein the platinum drug is cisplatin;
(b) the inner ear cell is a hair cell, optionally wherein the inner ear cell is at least one of a Deiter cell, Hansen cell, Claudius cell, primary afferent neuron, supporting cell, limbus cell, spiral ligament cell, type I vestibular hair cell, and/or type II vestibular hair cell;
(c) expression of the SOD protein protects the cell from oxidative damage;
(d) the AAV vector is targeted to the inner ear cell;
(e) the AAV vector comprises a capsid protein derived from an AAV2, optionally wherein the capsid protein is AAV2-7m8;
(f) the AAV vector comprises a capsid protein derived from an AAV9;
(g) the AAV vector is administered via middle ear injection, round window diffusion, round window injection, oval window injection, labyrinthotomy injection, intracochlear electrode or drug delivery system, oral, inhalation, nasal, nebulization intravenous injection, intramuscular injection, intrathecal injection, intrapleural delivery, cisterna magna injection, subcutaneous injection, or transdermal injection;
(h) the method further comprises administering to the subject an effective amount of an agent that disrupts the blood brain barrier;
(i) the SOD protein is selected from the group consisting of SOD1, SOD2, and SOD3.

47-57. (canceled)


SEQUENCE LISTING (12 sequences)

SEQ ID NO: 1 - DNA, 895 bp, synthetic construct
  source 1..895: mol_type = other DNA
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 2 - DNA, 14167 bp, synthetic construct
  source 1..14167: mol_type = other DNA
actcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagttaaaggcaaaattgtaaatcagtcgagatcgggtgccttcagggtggtatggctgtataccaaaattgtaaatcactacatgaagcttatatattggtttggcctgaaaggtgaagtggggtaggcagggggcgggcttacaggttatggtggattcaaagactccctgatttgtgattggttaaggaagcaaagctttgtctaaaaacttggggtccgcagaaaggaacattaaggtctggccaggcccctcaggaagaaactgagagcaaagaatggaggtcagagtttagtccctggtgttcccccttatctgacgtctgtgtgaatccatttggtgggggtctgggtttctgaaaagtagctcaggggcacgtgttaaggatgtctctaggtgactctaacttccctggctattgtttgaaactgttatgaccttcttgcttatcagcttgctggtttccttctcggggcgagctgggtgcctggagttttcggtgaaggaaactcaagattctcctttatttctgtgcttgtgggaatccccctggcacaccccaaagaggggtccctgctccgtctcacagggatctttttgtatatttggcttagcatcatacatttgccatgttgtttcatcatctgcctaatttactgtttttgaatatttcatttgtttctaattgttactacagataatgctggggtgagcaactctgtgtacataggtttatctcctattggaatattttctttatataggcgttttttttttttctttttttttggagacagagtcttgctctgttgcccaggctggagtgcagtggcgcgaccggagctcactgcaacctccacttcccgggttcaagtgattgtcccacctcagcctcctgaatagctgggattacaggtgcatgctaccatgcctggctactttttgtatttttagcagagacagggtttcaccatgttggccagggtggtctcgaactcctgacctcaagtgatccgtctggctcagcctcccaaagtgctgggattacaggtgtgagccactgcacctggcctatataggcttttttcttaaacctatttagtaatgttttcccaagtttattttttatttttaattttttccccaagtttatttttctatttttttttcatggaaaaatggggtaacttagcagtttcaatattgaagactgaagtttaaaaaaaatttaaattcaaggtacttttaaaattcagttagaaaagtaggctttaaaaattattagagacaagagtaccaaagcggtgtgtgtatgtgtgtgtgtgtatgcatgcttgtggattggaaaaactttggagactgattacttttcattatatatgtgtcacagtgaaacagcttttatgtgtcatgtaagattactgcttgcctctctaaggaaggtcgtgactgtttaaatagacgggcaaggtggaaccttttgaaagatgagcttttgaatataagttgtctgctagatcatggtttgtattgaactaacaaggtttgcagatctgctgacttatataaagctttttgattcctactaagctttaagatttaaaaaatgttcaatgttgaaatttctgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcaagaccagcgtggccaacgtggtgaaaccctgtctctactaaaaatacaaaaattgtttagctctgtttttcataatagaaatagaaaaggtaaaattgcttttcttctgaaaagaacaagtattgttcatccaagaagggtttttgtgactgaatcagcagtgcctgccctagtcatagctgtgcttcaaaaacctcagcatgattagtgttggagcaaaacaaggaagcaaagcaaatactgtttttgaaattctatctgttgcttgaactattttgtaataattaaactttgatgttgagaaatcacaactttattgtacacttcattgcaacttgaaattcatggtcttaaagtgagatttgaatttctattgagcgcctttaaaaaagtaataccaaaccataaagttaaaatctatgtatattgagtcatatctaaaaccacgtataaacataaattgtatttcctgttttaattccaggggaagtactgtttgggaaagctattattaggtaaatgttttacaaattactgtttctcactttcagtcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaagaggtctgcattatgcttgcatgtcaaaaactttaaaaatcctataatcttctgtcattttcactgagtttccatgggaaaggaatagtaaataatgggtagttgaaatattactcttaagaccaagacctgtatctccagtcatatctgtaataacatcatctgataacctaaaagcatagtattagggatatacgacaaaaccaaagtgtttttgctgttgtcacataccactcaatacttttacaccaggttgtccagtggacaccagctggatgtcctataattcaattctgacactgtctacctggaattggagtcagatcccacaggtggagggctcagcccacaagactgcccccactccagataccaatcgcaagtccctatacttctgactggctataaagtggggttcccgtgaccctttccttggttttgatttatttgctagagcagctcccagaacacagagaaacactctacttatgtttacccatttttaattttattactttattaaaactacatattggttacaatactaatttattgtaaaggatatttcatttatttatttttttatttagagatggagtctcactgtgtcacccaggctggagtgcggtggcacagtctcaactcactgcaacctccacctcctgagttcaagcgattctcatgcctcagcctcccaagtagctgggattacaagtgtgcaccaccatgcccagctaatttttgtatttttagtagagatggggtttcactctgttggccaggctggtctcaaactcctgacctcaagtcatccacccacctcagcctcccaaagtgctgagattacaggtgtgagccattgcctctggcttgtaaaggatatttcaaagaatacagatgaacagccagatggaagagatgcacagggcaaggcatgtgggaaggggtatggagctttcatgctctctccacgtcatcagctacccagaagctcatctggaccccgaccctttggggttttacggaggcttcattatgtcagcattaccattggcaattggcgattaactcaaccttcagcctctctcccctttctggaggttgggggttgaggctgggagtccaaaccctccaatcttgccttggtctttctggtaagcagccccccgtcctgaagctacctagaggctcccagccaccaatcatttcattggcaaaccaaagatacttcaaggggtttaggagccaagagaccaagcaaggattgagacaatatatatatttcttataaataacatgatcacaaatagtattttatatatttctcataatagctggttgtggttactcacacctatcatcccagctactaagaagctaaggcaggaggatcacaggcccaggagtttgcagctgtagtgagctatgattgtgctactgcactccagcctgggtgacagtgtgagaccctgtctcttaaagttcttgtaacatcttagaacattaaccaaccacagattattgccttaactctgaaaaataaagtaatcatacttttacaagatgtattttacataaagttctggtgatattttactctggtcattggaatataggagcttaccacctggtaccactcttcctaaagtggtatactatttggtatagtactggcatactatttggaaagggatgtgaagttggcatgacctgtctagggtgaagtcatgctgtctgctattcattgtgggctttacagtaatgagagtgcttctgttgtcaagatgcttctgggacacgttaaaaaaaatttaagatttctcagtttgtgaaaaggaaggagcacgaatgctttcaagttcctatgacatgctagtcatctttcagtgtgtcccattgttgcacccagtagtctcgatgcccagcgtcacggtgattccaagtttgcctctctgcccactataatttgtgctccatgaagtcccaggacatgaggttcttgttcatttgtgcgtctgggcccatatccaaatattaaacaatgtgacttaatgtatttatcatattgtaaatatgggtatccccaaggctcagaagttaacacagcatatccacagagaggtttgacagcatggctgtggtgactgggaaggccttatgcttgctcctccttctgctgcttgcctttgtgtgggcatggaaagaacattgaattaggtactggaagacctgggtcctggtcttggagattttaccagagcacctgtgggatattggtcaagatgcctggttttagtcctgctcctttgctgtgaaagaggatcttaaggtgccaactctggcattttattttttacttatttatttttaaattatactttaagttctagggtacatgtgtacaacatgcaggtttgttacatgtatacatgtgccatgttggtgtgctgcacctattaactcgtcatttacattaggtatatctcctaatgctatccctcccccttctccccacccctcgacaggccccggtgtgtgatgttccccttcctgtgtccaagtgttctcattgttcaattcccatctatgagtgagaacatgcggtgtttgtttttttgtccttggaatagtttgctgagaaggatggtttccagcttcatccatgtccctataaaggacatgaactcttccttttttatggctgcatagtattccatggtgtatatgtgccacatcttcttaaaccaatctatcattgatggacatttgggttggttccaagtctttgctattgtgaacagtgccacaataaatatacgtgtgcatgtgtctttatagcagcatgatttataatcctttgggtatatacccagtaatgggatggctgggtcaaatggtatttctagttctagatccctgaggaatcgccacactgtcttccacaatggttgaactagtttacagtcccaccaccagtgtaaaagtgttcctatttctccacatcctctccagcacctgttgtttcctgactttttaatgattgacattctaactggtgtgagatggtatctcattgtggttttgatttgcatttctctgatggcccatgatgatgagcatttttttcatgtgtctgttggatgcatggatgtcttcttttgagaagtgtctcttcatatccttcacctactttttgatggggtttgtttttttcttgtaagtttgtttgagttccttgtagattctggatattagccctttgtcagatgagtagattgcaaaaattttctcccattttgtaggttgcctgttcactctgatggtagtttcttttgctgtgcagaagctcttgagtttaatgagatcccatttgtcaattttggcttctgttgccattgcttttggtgttttagacatgaagtccttgcccatgcctgtgtcctgaatggtactgcataggttttcttctagggtttttatggttttaggtctaacatttaagtctttaatccatcttgaattaatttttgtataaggtgtaaggaagggatccagtttcagctttctacatgtggctagccagttttcccagcaccatttattaaatagggaatcctttccccatttcttgtttttgtcaggtttctcaaagatcagatagttgtagatgtgtggtattatttctgagggctctgttctgttccattggtctatatctctgttttggtactagtaccatgctgttttggttactgcagccttgtagtatagtttgaaatcaggtagcgtgatgcctccagctttgttcttttggcttaggatcgttttggcagtgcaggctcttttttggttccacatgaactttaaagtagttttttccaattctatgaagaaagtcatgggtagcttgatggggatggcattgaatctataaattaccttgggcagtatagccattttcacgatattgattcttcctatccatgagcatggaatgttcttccatttgtttgtgtcctcttttatttccttgagcagtggtttgtagttctccttgaagaggtccttcacatcccttgtaagttggattcctaggtattttattctctttgaggcagttgtgaatgggagttcactcatgatttggctctctgtttgtctgttgttggtgtataagaatgcttgtgatttttgcacattgattttgtatcctgagattttgctgaagttgcttatcaaattaaggagattttgggctgagatgatggggttttctaaatataaaatcatgtcatctgcaaacagggacaatttgacttcctcttttccttattgaataccctttatttctttctcctgcctgatggccctggccagaacttccaacactatgttgaataggagtggtgagagagggcatccctgtcttgtgccagttttcaaagggaatgcttccagtttttgcccattcagtctgatattggctgtgggtttgtcatagatagctcttattattttgagatatgtcccatcaatacctaattcattgagagtttttagcatgaagggctgttgaattttgtccaaggccttttctgcatctgttgagataatcatgtggtttttgtctttggttctgtttatatgctggattacttttattgatttgtgtatgttgaaccagccttgcatcccagggatgaagcccacttgatcatggtggataagctttttgatatgctgctggattcagtttgccagtattttattgaggatttttgcattgattttcatcagggatattggtctaaagttctctttttttgttatgtctctgccaggctttggtatcaggatgatgctggcttcataaaatgagttagggaggattccctctttttctattgattggaatagtttcagaaggaatggtaccagctcttccttgtacctctggtagaattcggctgtgaatccatcttgtcctggactttttttggttggtaggctattaattattgcctcaatttcagagcctgttattggtctattcagggattaaacttcttcctggtttagttttgagagggtgtatgtgtccaggaatttatctatttcttctagattttctagtttatttgcatagaggtgtttatagtattctctgtgtactctgtatttctgtgggattggtggtgatatccactttatcattttttattgcatttatttgattcttctctcttttcttgtttattagtcttgctagcggtctatcaattgtgtttatcttttcaaaaaaccagctcctggattcattcattttttgaaggtttttttgtgtctctgtctccttcagttctgctctgatcttagttatttcttgtcttctgctagctgttgaatgtgtttgctcttgcttctctaatatttttaattgtgatgttagagtgtcaattttagatctttcctgctttctcttgtgggcacttagtgctgtaaatttccctctacacactgctttaaatgtgtcccagagattctggtatgttgtgtctttgttctcgttggtttcaaagaacatctttatttctgccttcattttgttatgtacccagtagtcattcaggagcaggttgttcagtttccatgtagctgagtggttttgagtgagtttcttaatcctgagttctagtttgattgcactgtggtctgagacagtatgttataatttctgttcttttacatttgctgaggagtgctttacttccaactatgtggtcaattttggaataggtgcgatgtggtgctgagaagaatgtatattctgttgatttggggtggagagttctgtagatgtctattaggtctgcttggtgcagagctgaattcaattcctggatatccttgttaactttctgtctcgttgatctgtctaatattgacagtggggtgttagagtctcccattgttattgtgtgggagtctaaatctctttgtaggtctctaaggacttggtttatgaatctgggtgctcctgtattgggtgcatatatatttaagatagttagctcttcttgttgaattgatccctttactatcatgtaatggccttctttgtctcttttgatctttgttggtttaaagtctgttttatcagagactaggattgcaaccccggcctttttttttgttttccgtttgcttggtagatcttcctccatccttttattttgagcctatgtgtgtgtgtctctgtacgtgagatgggtttcctgaatacagcaccctgatgggtcttgactctttatccagtgtgccagtctgtgtcttttaattggagcatttagcccatttacatttaaggttaatattgttatgtgtgaatttgatcctgtcactaggatgttcactggttattttgctcgttagttgatgcagtttcttcctagcctcaatggtctttacaatttggcatgtttttgcagtggctgatactggttgttccattccatgtttagtgcttccttcaggagctcttgtaaggcaggcctggtggtgacaaaatctctcagcatttgcttgtttgtaaaggattttatttctccttcacttatgaagcttagtttggctggatatgaaattctgggttgaaaattcttttcttcaagaacgttgaatattgacccccactctcttctggcttgtagagtttctgctgagagatcagctgttagtctgatgggcttccctttgtgggtaacccgacctttctctctggctgcccttaacattttttccttcatttcaactttggtgaatctgacaattatgtgtcttggagttgctcttctcgaggagtatctttgtggcgttctctgtatttcctgaatttgaatgttgtcctcccttgctagattggggaagttctcctggataatatcctgcagagtgttttccagcttggttccattctccccgtcactttcaggtacaccaatcagacgtagatttggtcttttcacatagtcccatatttcttggaagctttgttcgtttctttttactcttttttctcttaacttctcttctcacttcatttcattcatctgatcttcaatcactgataacctttcttccagttgatcgaattggctactgaagcttgtgcattcgtcacgtagttctcatgccatggttttcagctccatcaggtcatttaaggacttctctatgctggttattctagttagccatttgtctaatcttttttcaaggtttttaggttctttgtgatgggttcgaacttcctcttttagcttggagaagtttgatcatctgaagccttcttctctcaactcgtcaaagtcattctccgtccagctttgttctgttgctggcaaggagctatgttcctttggagggggagaggcgctctgatttttagaattttcagcttttctgctctgttttttccccatctttgtggttttatctacctttggtctttggtgatggtgatgtgcagatggggttttggtgtggatgtcctttctgtttgttagttttccttctaacagtcaggaccgccagctgcaggtctgttggagtttgctggacgtccacgccagaccctgtttgcctgggtatcacagtggaggctgtagaacagcgaatattgctgaacagcaaatgttgctgcctgatcattcctctggaagcttcatctcagaggggtacccggccatgtggggtgtcagtctgcccctacttgggggtgcctcccagttaggctactcgggggtcagggactcacttgaggaggcagtctctccgttctcagatctcaaactctgtgctgggagaaccactggtctcttcaaagctgtcagacagggacattgaagtctgcagagatttctgctgccttttgttcggctatgccatgcccccagaggtggagtctacagaggcaggcaggcaggcctccttgagctgcagtgggctccacctagttcgagcttcccggctgctttatttacctactgaagcctcagcaatggtgggctcccctcccctagcctcactgccaccttgcagttcgatctcagactgctgtgctagcaatgagcaaggctccatgggcgtgggaccctccgagctaggcacaggatataatctcctggtgtgccgtttgctaagaccattggaaaaacgcagtattagggtgggagtgacccaattttccaggtgccgtctgtcacagcttcccttggctaggaaagggaattccctgaccccttgccttcccgggtgaggccatgccttgccctgcttcagctcatgctcagtgggctgcacccaccgtcctacacccactgtccaacaagccccagtgagatgaacctggtacctcagttggaagtgcagaaatcacctgtcttctgcgtcgctcacgctgggagctgtagactggagctgttcctattcggccatcttggaacccaacccaagtctggcattttttggttttctctgtgtattgactatgattatagaggataaatggatattgatattattaaatcgtcatttacagaaggcacacaacttatatcctttaaacaaagttctccaaccagtagggtatacttgctggtagtgcttacagctgccaccattcttcactggggctcagagtggatgcctggaggaaaaaaacaaatgaatcttttagctcctagttactgttagcatttttaccagaaaacaatttaaaaaatgacacaagtaagtcttcacctatcaaggctgagtgtggtggcctatacttgtaatctctgtgctttgggaggccaacgcaggaggatctcttgagtccaggagtttgagaccagcctgggcaacaaagcaagaccccatctctacagaaagttaaaatttagctgggcatgctggcacatccctatagtcccagctacttgagaggctgagttgggaggatcccttgaggtcatgagttggagtctacagtgagctgtgattacagtgtgagtctacactgcactccagtctgggtgaaagagatcctgtctctcaaaaaacaagtgattctcctgggaagagatttgtttaaaaagtctgcacctctattgatgataactttgaatgtaaatggattaaattctcccacttaaaagatatagattgactgaatggattaaaaaccaccatcatgactcgactgtatgctgcctacaagaaacctcacctgtaaagacaggccaaaagtgaaggtatggagaaagatattgcatacaaatggaagccaaaaaagagcagaaataactatacttacatgagataaaatagactttaagtcaaaaactgtaaaaagaaaagtcattatgtaatgaaacaaggatcaattcatgcaagatataatcatatttttttattaagaaaatttttttgtagagatggggttatgctatgttgcctggctagtctcaaactctgggtctcaaagcagtcctcctgctttggcctcctaaggtgctgggattataggcatgagccactgtgccaggcctgaggatattatcattataaatatatatgtaccccacactggatgacccagatataaagcaaatattattagatttaaagggagaaatagactctaatacaatagtagttgaggacttcactctactctcagcattggacagattttctagacataaaaccaataaatattggatttaaattgcactttacaccaaatggacctgacatttacagaacattctaataggtgcagaacactctaatagctgcagaacacacgttcttttcatcagcacatggaacattcaccagactagttctggaacccacttcctgccttagtgttctttccaccacagtgcatgataattccgacagaacgccttttatttgtaccttattagttgtagggggaaagctttttcatgtacaaaaatgctgtaattttctgatttaataaagtaattccactga

SEQ ID NO: 3 - DNA, 1416 bp, synthetic construct
  source 1..1416: mol_type = other DNA
agggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcgcgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgagcgcggcccccacccggcggcggccagggacccccgaggcccccctctgcctttgagcttctcctctgctccaacagacaccctccactctgaggtctcaccttcgcctttgctgaagtctccccgcagccctctccacccagaggtctccctataccgagacccaccatccttccatcctgaggaccgccccaaccctcggagccccccactcagtaggtctgaaggcctccatttgtaccgaaacaccccgctcacgctgacagcctcctaggctccctgaggtacctttccacccagaccctccttccccaccccataagccctgagactcccgcctttgacctgacgatcttcccccttcccgccttcaggttcctcctaggcgctcagaggccgctctggggggttgcctcgagtccccccacccctccccacccaccaccgctcccgcgggaagccagcccgtgcaacggaagccaggccaactgccccgcgtcttcagctgtttcgcatccaccgccaccccactgagagctgctcctttgggggaatgtttggcaacctttgtgttacagattaaaaattcagcaattca

SEQ ID NO: 4 - DNA, 8369 bp, synthetic construct
  source 1..8369: mol_type = other DNA
gaattcccatcatcaataatataccttattttggattgaagccaatatgataatgagggggtggagtttgtgacgtggcgcggggcgtgggaacggggcgggtgacgtagtagctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgtgagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctatgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaaacaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccccagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagatacctgactcgtaatctgtaattgcggccgcttgttaatcaataaaccgtttaattcgtttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcatcgctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcatattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgca

SEQ ID NO: 5 - DNA, 1120 bp, synthetic construct
  source 1..1120: mol_type = other DNA
gcgttacataacttacggtaaatggcccgcctggctgaccgcccaacgacccccgcccattgacgtcaataatgacgtatcgcaatgtattgaatgccatttaccgggcggaccgactggcgggttgctgggggcgggtaactgcagttattactgcatagttcccatagtaacgccaatagggactttccattgacgtcaatgggtggagtatttacggtaaactgcccacttggcagtcaagggtatcattgcggttatccctgaaaggtaactgcagttacccacctcataaatgccatttgacgggtgaaccgtcaacatcaagtgtatcatatgccaagtacgccccctattgacgtcaatgacggtaaatggcccgcctggcattatgcccagttgtagttcacatagtatacggttcatgcgggggataactgcagttactgccatttaccgggcggaccgtaatacgggtcaacatgaccttatgggactttcctacttggcagtacatctacgtattagtcatcgctattaccatggtcgaggtgagcccctgtactggaataccctgaaaggatgaaccgtcatgtagatgcataatcagtagcgataatggtaccagctccactcggggacgttctgcttcactctccccatctcccccccctccccacccccaattttgtatttatttattttttaattattttgtgctgcaagacgaagtgagaggggtagaggggggggaggggtgggggttaaaacataaataaataaaaaattaataaaacacgagcgatgggggcggggggggggggggggcgcgcgccaggcggggcggggcggggcgaggggcggggcggggcgaggcggatcgctacccccgccccccccccccccccgcgcgcggtccgccccgccccgccccgctccccgccccgccccgctccgcctgaggtgcggcggcagccaatcagagcggcgcgctccgaaagtttccttttatggcgaggcggcggcggcggcggccctatctccacgccgccgtcggttagtctcgccgcgcgaggctttcaaaggaaaataccgctccgccgccgccgccgccgggata

SEQ ID NO: 6 - RNA, 692 nt, synthetic construct
  source 1..692: mol_type = other RNA
ccgcggcgccggggtctcggtttgcgccggcgtctcctgcggcggcttgtggcgtcgcttgctctcccagcgctccctcaggttcccgaggccgtcgcgcgtgtcccggcgaagcatggcgatgaaggccgtgtgcgtgctgaagggcgacggtccggtgcagggcaccatccgcttcgagcagaaggcaagcggtgaaccagttgtggtgtcaggagaaattacaggattgactgaaggccagcatgggttccatgtccatcagtttggggataatacacaagggtgtaccagtgcaggacctcattttaatcctcattccaagaaacatggcggaccagcagatcaagagaggcatgttggagacctgggcaatgtgattgctggaaaggatggtgtggccaaagtgtccattgaagatcgtgagatctcactcacaggagagcattccatcattggccgtacaatggtggtccatgagaaacaagatgacttgggcaaaggtggaaatgatgaaagtacaaagactggaaatgctggaagccgcttggcttgtggtgtgattgggattgcccagtaaacaaacattccctctgtggtctgagtctcaaactcatctgctgtcctgctaagctgtaggaaaaaataaacattaaactaatctttaacagtgtaactgtgtgactcctttt

SEQ ID NO: 7 - DNA, 794 bp, synthetic construct
  source 1..794: mol_type = other DNA
gacacgtgctgaagtcaagtttgaaggtgatacccttgttaatagaatcgagttaaaaggtattgattttaaagaagatgctgtgcacgacttcagttcaaacttccactatgggaacaattatcttagctcaattttccataactaaaatttcttctacgaaacattcttggacacaaattggaatacaactataactcacacaatgtatacatcatggcagacaaacaaaagaatggactttgtaagaacctgtgtttaaccttatgttgatattgagtgtgttacatatgtagtaccgtctgtttgttttcttacctatcaaagttaacttcaaaattagacacaacattgaagatggaagcgttcaactagcagaccattatcaacaaaatactcctagtttcaattgaagttttaatctgtgttgtaacttctaccttcgcaagttgatcgtctggtaatagttgttttatgaggaattggcgatggccctgtccttttaccagacaaccattacctgtccacacaatctgccctttcgaaagatcccaacgaaattaaccgctaccgggacaggaaaatggtctgttggtaatggacaggtgtgttagacgggaaagctttctagggttgctttagagagaccacatggtccttcttgagtttgtaacagctgctgggattacacatggcatggatgaactatacaaatagtctctctggtgtaccaggaagaactcaaacattgtcgacgaccctaatgtgtaccgtacctacttgatatgtttatc

SEQ ID NO: 8 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 9 - DNA, 623 bp, synthetic construct
  source 1..623: mol_type = other DNA
ggtgtggaaagtccccaggctccccagcaggcagaagtatgcaaagcatgcatctcaattagtcagcaaccaggtgtggaccacacctttcaggggtccgaggggtcgtccgtcttcatacgtttcgtacgtagagttaatcagtcgttggtccacacctaagtccccaggctccccagcaggcagaagtatgcaaagcatgcatctcaattagtcagcaaccatagtcccgcccctaacttcaggggtccgaggggtcgtccgtcttcatacgtttcgtacgtagagttaatcagtcgttggtatcagggcggggattgtccgcccatcccgcccctaactccgcccagttccgcccattctccgccccatggctgactaattttttttatttatgcagaggcgggtagggcggggattgaggcgggtcaaggcgggtaagaggcggggtaccgactgattaaaaaaaataaatacgtcaggccgaggccgcctcggcctctgagctattccagaagtagtgaggaggcttttttggaggcctaggcttttgcaaatccggctccggcggagccggagactcgataaggtcttcatcactcctccgaaaaaacctccggatc

SEQ ID NO: 10 - DNA, 3655 bp, synthetic construct
  source 1..3655: mol_type = other DNA
tcgccgtcctcccctccgccgatgggagcggccggcagggtcgccgccctgaagtgtccttgtagaggcgcctcggacgccgcggagcagacgcacgcctgcgagcggaccgttgacttgtccggacaccggaggccgtgggcatctcagcaatgctgtgtcgggcggcgagcagcgcgggcaggaggctgggcccggcggccagtgtcgcgggctcccggcacaagcacagccttcccgacctgccttacgactatggcgcgctgcagccgcacatcgacgcgcagatcatgcagctgcaccacagcaagcaccacgcggcctacgtgaacaatctgaacgtcaccgaggagaagtaccacgaggcactggccaagggagatgttacggctcagattgctcttcagccagcactgaggttcaatggtgggggccatattaatcactccatattctggacgaacttgagccctaatggtggtggagaacccaaaggagagttgctggaggctatcaaacgtgactttgggtcttttgagaagtttaaggagaagctgacagctgtgactgttggaatccaaggttcaggctggggctggcttggcttcaacaaggagcagggtcgcttgcagattgctgcctgcgctaaccaggatccattgcaaggaacaacaggccttattccattgctggggattgatgtgtgggaacatgcctattaccttcagtataaaaacgtcagacccgattacctgaaagctatttggaatataatcaactgggagaatgtcactgaaagatacacagcttgcaagaagtaaagcctcatcaccatattgagcatatcaggcccttgatggctacttttgtagtagtataaagtgctgcagctactgtgactgcgctgttgttgacatttattgatgtacgtccacatagctgatgactaggatgttaagataatttcttattcttaattaaacttacaattaggcaactgtttgagaacagtacatattctgtgtgaattattcttgattgaacattttcatttgaaccttgaattgctaggatgctctgtcattgtaagactaccaaaaatattctatctcaatgttttgggggcctactggagagtctaatcctgttctactgcagttagaaaaaaaatggagttgttcccccacctcgagttgttaaacaataaaatagttcattttctattaaaaattgctatttttggtaagtaagcctttgcttagtagatatcacctagtggtctttatttatggtcacagttctgcatgagaaacacaatttttttatttgaaatctgtaacttgaggctaaggatgtagttcagtggtagagctttagtgtgcaaagccctgggttctacttcagtactgaaaacaaatcaacagaacccatagcttggttactgagtgagaactgttttattataagaggagtctaattgtgcctctggggtttttctataggcagagatggaaaaacatttaatattgtctttaaaaaccaattgtatgaagtgttcaacgtggactattagggcctgcctgatgatgtcaaccatggggactgggccacagggcattctgggaagccacctagcccccttaattgtgctggagaactggaccctgggccagatggcacatttctcagtagaggcaaatggatttctgagtgaagttagaagggtgtcttcagaacattagttttcagagacatttatttttattttatctatagtttcaaaattttatagctaccagaggggctttatattatgttcttcagtaaagggagggactttccatgacaagatgacacagtgtttctgattgtgttcctgtgcagagttgtatgtggagttgtgccctgtggaaatgccacaggggcccttgtgaatttgcctttcatattttgttttagcatcttcctaactgtctttaatgactatgcaatgttttaatcaattggacatgccatttatagttccattactgggtagttcatttgtgcttaattgttgtttattaccaaaaaggctgtgtcagcaaccccatgcatttgaagtacttcttactgattggggtcttttttaaggttcatcttatttgcagttatgtgtgtctgtgtatgtctgttcatatgaatgcaggtgcccatggaagacagcagatagtgttggatctctcagagcggaagtagttacagtggccgaagccacctgatgtgggtgtgggacttggactggggtcacctgcaagagcaataagcacttaaccactgagccacctctccagcccctgcattttttctttctttttctttttctttttttttggttaaatactgagtctttttttttcttcttaaatctatcttgtaatttattccaagttttttttaatcctcccctcaagtatattttccttttttttttaatggaaaaatggagtgacttagaagtttcagtattgaagactgaagtttaaaaaacaaaaccaaacttaaattcaaagcactttataacagaagctcatcaggtgttttcagcgtcagtgctgatcggtcctggtggctccttttgtatgtttatacttgtgtgtggagggactcagaagactgagtagcagccagttgtatgggacagtgacaggactgagcaaatgagagtcaacatggttccactttgtttacaaggcagtgaactcttttaaacagactttttaggaaagtgggactttgatggatagactttggaaaacaagtcttaagatcagttttcttgaactaacaaagatttgcagagactactgactttataaaactttgactcatactaagctttcagattaaaaaaaattcaatggtgaaatttctatgaggctctatttttgctgtggctttctgttgtcaagaatgaggaatggcaccgtgttcattcggttaaagtctcccgatgcatagatttttaagagaccaggagaatttacaaactaaaataatccagtgtaaaataaggcttagattgggaagaaatatgtgttaagtgtgtgtgagtatgtgtgagtgtgtgcaacaagatcagcagtgtgttgtcccagtcatggttgaactgacaggcatcagcatatactggggagaaacaaggaagctatttcctttgaggtagtatctgctgcttgaactattgtgcattaattaaactttggagttaagaaattgccttattgcattctgaaattcttggcctacagtgagatctgaatttctgttaagcacctttataaaaagtattgaagccatatagtcaggactgatatgtattgtgtgtggtgtcatagtgaaaactaggtataaatactggctttttttttcctgttgtaattcagttaaactgctttcaggaacactgttaggtacgtttttaaataagtcacagcaaagcatatcctgctgatcccaggaagctagcatttgtcttctgaacatcatgtctagcacctgctgaaaagtctgtaaggtagttttcgcttaaatattgatcaaagaagtgtttattttcctattgtaagtgcaaagtggtgttgattgttaataaaaatagtctgtcaactatcaa

SEQ ID NO: 11 - DNA, 2027 bp, synthetic construct
  source 1..2027: mol_type = other DNA
gggttatgtacctcggacaacctcagacagaaattacaaaactcacacaaaaatcctaccccatgtctttgctgaacatatacaggcctttcttcttgtgatggctatttacatagcatttacactgctaacttcaataagccgtccaggcatgttttaaaggacacagggccatctgagctgtttctgggcacagttcttgctatcttacataaggaacttgggcacccatgaattttggaatccttgaagcatcctggaatcagtcacccacatatgaagtgagtcagctgtgttgtgactaaggttgaattctggacctttccattgtctttgattgcgcaccaggatatttaaactttcaaaaaggcaaacgacagctaagctcttctgaatccaggctctcgggtggtggcctcttgtggatcttggttttagagtcttggatccaggctgggaagatggactggtgcaggacctcagccatgctggccttcctgttttgtggcttgcttctggcggcccatggctctgtcacctggaccatgctggatcccggggagtccagcttcgacctagcagagatcaagactgacctgtttgagaagataagcgacactcacgccaaagtgctggagatctggctggggctgggacggcgggaggtggatgcccctgagttgtacgcggcctgcagggtggagccgtcagccacgctggcagccgatcagcctcgggtcaccggtctggtcctcttccggcagctagcgccgggcgccaagctggaggccttcttcaatctggagggcttcgatgacgagctgaacgtgtccagccacgccatccacgtgcacgagttcggggacctgagccagggctgtgagtccaccgggccgcactacaacccgctggccgtggcgcacccgcagcacccgggcgacttcggcaacttcgtggtgcgcgatgggcgcctctggaagtaccgctctggcttggtcgcttcgctggcgggcccgtactccatcttgggccgtgccgtagtggtccacgctggccaggatgacctgggccgcggcggcaaccaggccagcgtggagaacggcaacgcggggcgccggctcgcctgctgcatagtgggtgccagcagctctgcggcctgggagcgccaggccaaggagcacaccgagcgtaaaaagcggcggcgggagagcgagtgcaagaccacctaagcggcactcccagccgcagggcccagctgcttcacgcatagatgcctccacaggtcccggacaccaccctcactccagaggcctccacagtcctagacagatgccttccagacgccacagtcgcctctgcgcgccccacatgcttccaggcactccaggcacccctgtgtgctcccagacggcttcacggaactccccagtcacctctcggtgccccatttgttcccacgtgccctcgacacccttcctgtgtgtcccaggacagcttgagtaacccaggaaacttttcatgccctaggcactttcacagacccagaactccttcataccccagacccacctcaagagttcccctgtgtcccaagcctttgaaagagtctttgagtctgtttgctgccggagaaccccctctccccaggctgcacgtgctcagacgctccccttccacgctgaggacctcgatggcagcacctgagtacatcctcctcagttgtgcggaaatccatttcctcctgtccatcttttccttcctatcccccagcaaccgacacgagggactttttttcccttttgttcctcctagatgcccagagaccatcccaacacacacacacacacacacacacacacacacacacacacctaggattccatgtcccacaccacctcctgcggtgcccccggctcgcttttcagctgtttcccacatggtgcctgcaccctgtgcagagaggctcccatgagagtacttggcaacctttgtgccgtacattaaaaacacagcaattcagtcctgca

SEQ ID NO: 12 - DNA, 1690 bp, synthetic construct
  source 1..1690: mol_type = other DNA
gctgctcacaaacagccagccggccagccagccagccctggggaggcagcacataggttctccctcaggcttctagctgggtgctgccccccacttcgccagagggaaagcgctcttgggagagcctgacaggtgcaggacctcagccatgctggccttcctgttttgtggcttgcttctggcggcccatggctctgtcacctggaccatgctggatcccggggagtccagcttcgacctagcagagatcaagactgacctgtttgagaagataagcgacactcacgccaaagtgctggagatctggctggggctgggacggcgggaggtggatgcccctgagttgtacgcggcctgcagggtggagccgtcagccacgctggcagccgatcagcctcgggtcaccggtctggtcctcttccggcagctagcgccgggcgccaagctggaggccttcttcaatctggagggcttcgatgacgagctgaacgtgtccagccacgccatccacgtgcacgagttcggggacctgagccagggctgtgagtccaccgggccgcactacaacccgctggccgtggcgcacccgcagcacccgggcgacttcggcaacttcgtggtgcgcgatgggcgcctctggaagtaccgctctggcttggtcgcttcgctggcgggcccgtactccatcttgggccgtgccgtagtggtccacgctggccaggatgacctgggccgcggcggcaaccaggccagcgtggagaacggcaacgcggggcgccggctcgcctgctgcatagtgggtgccagcagctctgcggcctgggagcgccaggccaaggagcacaccgagcgtaaaaagcggcggcgggagagcgagtgcaagaccacctaagcggcactcccagccgcagggcccagctgcttcacgcatagatgcctccacaggtcccggacaccaccctcactccagaggcctccacagtcctagacagatgccttccagacgccacagtcgcctctgcgcgccccacatgcttccaggcactccaggcacccctgtgtgctcccagacggcttcacggaactccccagtcacctctcggtgccccatttgttcccacgtgccctcgacacccttcctgtgtgtcccaggacagcttgagtaacccaggaaacttttcatgccctaggcactttcacagacccagaactccttcataccccagacccacctcaagagttcccctgtgtcccaagcctttgaaagagtctttgagtctgtttgctgccggagaaccccctctccccaggctgcacgtgctcagacgctccccttccacgctgaggacctcgatggcagcacctgagtacatcctcctcagttgtgcggaaatccatttcctcctgtccatcttttccttcctatcccccagcaaccgacacgagggactttttttcccttttgttcctcctagatgcccagagaccatcccaacacacacacacacacacacacacacacacacacacacacctaggattccatgtcccacaccacctcctgcggtgcccccggctcgcttttcagctgtttcccacatggtgcctgcaccctgtgcagagaggctcccatgagagtacttggcaacctttgtgccgtacattaaaaacacagcaattcagtcctgca